(19) United States
(12) Patent Application Publication
(10) Pub. No.: US 2024/0228448 A1
(43) Pub. Date: Jul. 11, 2024

(54) COMPOSITIONS FOR BINDING SPHINGOSINE-1-PHOSPHATE RECEPTOR 1 (S1P1), IMAGING OF S1P1, AND PROCESSES FOR PREPARATION THEREOF

(71) Applicant: Washington University, St. Louis, MO (US)

(72) Inventors: Zhude Tu, St. Louis, MO (US); Lin Qiu, St. Louis, MO (US); Jiwei Gu, St. Louis, MO (US); Yanbo Yu, St. Louis, MO (US); Robert Gropler, St. Louis, MO (US)

(73) Assignee: Washington University, St. Louis, MO (US)

(21) Appl. No.: 18/553,411

(22) PCT Filed: Mar. 31, 2022
(86) PCT No.: PCT/US2022/022926

§ 371 Date: Sep. 29, 2023

Related U.S. Application Data
(60) Provisional application No. 63/169,039, filed on Mar. 31, 2021.
(60) Provisional application No. 63/288,336, filed on Dec. 10, 2021.

(51) Int. Cl. C07D 271/06; A61K 31/4245; A61K 31/4439; C07D 413/10

(52) U.S. Cl. CPC C07D 271/06; A61K 31/4245; A61K 31/4439; C07D 413/10

(57) ABSTRACT

The present disclosure generally relates to compounds and compositions for use in imaging agents, methods of use for monitoring and/or treating conditions or diseases related to sphingosine-1-phosphate (S1P) signaling, and processes for preparing these compositions and compounds.

### FIELD OF THE INVENTION

The present disclosure generally relates to compounds and compositions for use in imaging agents, methods of use for monitoring and/or treating conditions or diseases related to sphingosine-1-phosphate (S1P) signaling, and processes for preparing these compositions and compounds.

### BACKGROUND

Sphingosine-1-phosphate (S1P) is a natural metabolite of sphingolipids, which comprise cell plasma membranes. Aside from its role in intracellular signaling, S1P is also released and acts in an autocrine or paracrine fashion on a family of G-protein coupled receptors (GPCRs): Sphingosine-1 Receptors 1-5 (S1P1-S1P5 or S1PR1-S1PR5). S1P signaling has been linked to a variety of cellular processes including cell motility, invasion, angiogenesis, vascular maturation and lymphocyte trafficking. Recently, a modulator of S1P1 and S1P3-S1P5, fingolimod (also known as FTY-720 or Gilenya), has been found to be an effective treatment for relapsing-remitting multiple sclerosis (RRMS), a chronic neuroinflammatory disease. S1P1 is the primary target of FTY-720. Fingolimod promotes the internalization of S1P1, reducing the aberrant lymphocyte trafficking common in MS. The surface expression level of S1P1 can be used as a marker of several diseases, including multiple sclerosis (MS), cancer, cardiovascular disease and rheumatoid arthritis. S1P1 levels can be assessed using imaging techniques such as positron emitting tomography (PET), which uses radioisotope labeled ligands that bind to a target and release gamma rays that can be detected for localization and quantification.

The structure of S1P is below:

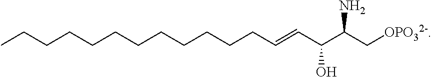

The structure of FTY720 (Fingolimod) is below:

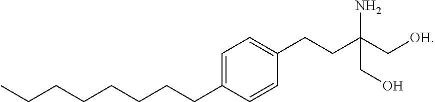 [SMILES: CCCCCCCCc1ccc(CCC(N)(CO)CO)cc1]

Previously identified structures of S1P1 radiotracers (Briard, E.; et al. ChemMedChem 2011, 6, 667; Shaikh, R. S.; et al. J. Med. Chem. 2015, 58, 3471; Rosenberg, A. J.; et al. J. Med. Chem. 2016, 59, 6201; Luo, Z.; et al. Org. Bio. Chem. 2018, 16, 9171.) are shown below:

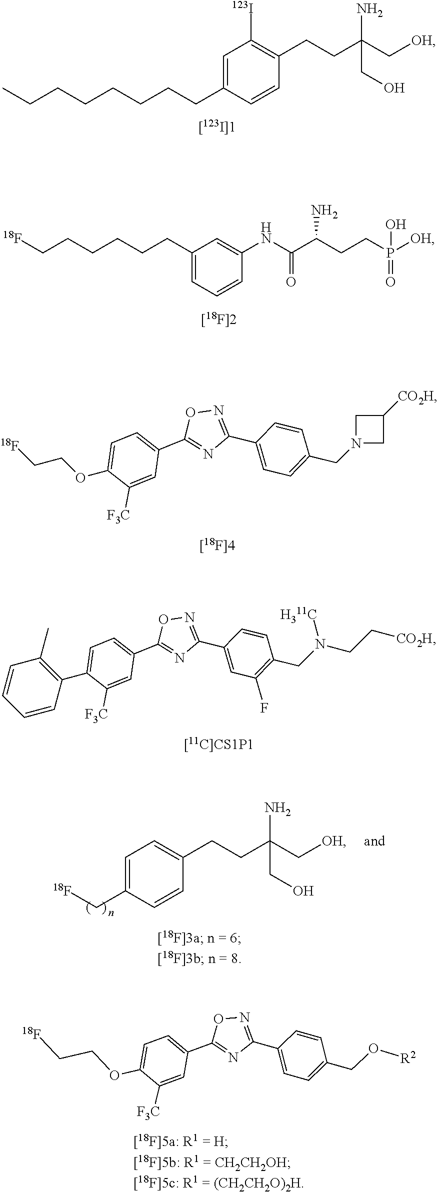

CS1P1 was identified as a key lead compound to optimize. Properties of an ideal CNS PET radiotracer include high binding affinity (IC50/Ki<20 nM), good selectivity (IC50/Ki>1000 nM for non-targets), capability of crossing Blood-Brain Barrier (BBB), favorable in vivo kinetics, and lack of accumulation of radiometabolites in the brain. CS1P1 has IC50=2.13±1.63 nM for S1P1 and >1000 nM for S1P2-5.

Given the association between S1P1 expression and signaling in various disease states, there is a need for new compounds having high affinity and selectivity for the S1P1.

Assessment of sphingosine-1-phosphate receptor 1 (S1P1) expression could be a unique tool to determine the neuroinflammatory status for central nervous system (CNS) disorders. Preclinical results indicate that PET imaging with [¹¹C]CS1P1 radiotracer can quantitatively measure S1P1 expression changes in different animal models of inflammatory diseases. Here a multiple step F-18 labeling strategy was developed to synthesize the radiotracer [¹⁸F]FS1P1, sharing the same structure with [¹¹C]CS1P1. A wide range of reaction conditions for the nucleophilic radiofluorination was explored starting with the key ortho-nitrobenzaldehyde precursor 10. The tertiary amine additive TMEDA proved crucial to achieve high radiochemical yield of ortho-[¹⁸F]fluorobenzaldehyde [¹⁸F]12 starting with a small amount of precursor. Based on [¹⁹F]12, a further four-step modification was applied in one-pot to generate the target radiotracer [¹⁸F]FS1P1 with 30-50% radiochemical yield, >95% chemical and radiochemical purity, and a high molar activity (37-166.5 GBq/μmol, decay corrected to end of synthesis, EOS). Subsequently, tissue distribution of [¹⁸F]FS1P1 showed a high brain uptake (ID %/g) of 0.48±0.06 at 5 min, and bone uptake of 0.27±0.03, 0.11±0.02 at 5, and 120 min respectively, suggesting no in vivo defluorination. MicroPET studies showed [¹⁸F]FS1P1 has high macaque brain uptake with a standard uptake value (SUV) of ˜2.3 at 120 min. Radiometabolite analysis in nonhuman primate plasma samples indicated that [¹⁸F]FS1P1 has good metabolic stability, and no major radiometabolite confounded PET measurements of S1P1 in nonhuman primate brain. Overall, [¹⁸F]FS1P1 is a promising F-18 S1P1 radiotracer worthy of further clinical investigation for human use.

Sphingosine-1-phosphate (S1P) is a natural high-affinity ligand that binds to the five members of the S1P receptor family (S1P1, 2, 3, 4, and 5). S1P1 is the most abundant of the five members of S1P receptors and is expressed in a broad range of tissues including the central nervous system (CNS). It plays a key role in many physiological and cellular processes. For example, it is involved in the activation of the immune response by regulating differentiation, egress, and migration of immune cells. S1P1 is widely accepted as a therapeutic target for treating inflammatory diseases, such as multiple sclerosis (MS), colitis, inflammatory bowel diseases, and atherosclerotic disease. To date, the mechanism of S1P1 modulation in CNS remains not fully understood. An S1P1 specific PET radiotracer may provide a unique non-invasive tool to advance the understating of S1P1 function in CNS and other diseases.

To identify a clinical suitable S1P1 specific radiotracer, the synthesis and evaluation of a carbon-11 labeled S1P1 radiotracer [¹¹C]CS1P1 was previously reported in three animal models of diseases including MS, carotid injury, and vascular injury. With FDA approval of an exploratory Investigational New Drug (eIND) of [¹¹C]CS1P1 for human use, dosimetry and safety studies of [¹¹C]CS1P1 were recently completed in 10 healthy volunteers (5 female and 5 male), suggesting [¹¹C]CS1P1 is safe for human use to investigate neuroinflammation (S. Mansor, et al., J. Nuc. Med., 2021, 62, 1591-1591.). The proof of mechanism study in MS patients is currently underway. Although C-11 labeled radiotracer confers many advantages such as low radiation exposure for patients and the short half-life of the isotope (20.38 minutes) permits multiple studies on the same subject in the same day but also constrains production and distribution for multicenter clinical trials using PET. On the other hand, F-18 labeling is most widely used in clinical PET imaging studies of cardiology, oncology, and neurology. Compared to C-11 radiotracers, the relatively long half-life of F-18 isotope (109.7 minutes) allows for the multiple step synthesis and longer scan sessions and F-18 radiotracers that can increase target-to-reference ratios. In addition, F-18 radiotracers facilitate radiotracer distribution for multi-center clinical trials. Therefore, identification of a clinically suitable F-18 S1P1 radiotracer is imperative. See table below comparing PET radionuclides and their properties.

TABLE 1
PET Radionuclides and Their Properties

| | | | | Mean B+ | |
|---|---|---|---|---|---|
| isotope | T_{1/2} | | Production | Energy (MeV) | B+ fraction |
| C-11 | 20.4 | min | ¹⁴N(p, a)¹¹C | 0.386 | 100% |
| N-13 | 10.0 | min | ¹⁶O(p, a)¹³N | 0.492 | 100% |
| O-15 | 2.04 | min | ¹⁴N(p, n)¹⁵O | 0.735 | 100% |
| F-18 | 109.8 | min | ¹⁸O(p, n)¹⁸F | 0.250 | 97% |
| Cu-64 | 12.7 | hr | ⁶⁴Ni(p, n)⁶⁴Cu | 0.278 | 18% |
| Ga-68 | 67.7 | min | ⁶⁸Ge generator | 0.830 | 89% |
| Rb-82 | 1.28 | min | ⁸²Sr generator | 1.48 | 95% |
| Zr-89 | 3.3 | days | ⁸⁹Y(p, n)⁸⁹Zr | 0.396 | 23% |

Although a few F-18 radiotracers have been reported for S1P1, none have been transferred for clinical investigation due to either high non-specific binding, fast metabolism in vivo, or other concerns. Inspired by promising preclinical animal study results, and human dosimetry, tissue distribution, and safety studies of [¹¹C]CS1P1 from whole body PET scans in healthy volunteer subjects, herein is reported a multiple step approach to incorporate F-18 through the aromatic ring of the CS1P1 structure that yields an F-18 labeled radiotracer, 3-((2-[¹⁸F]fluoro-4-(5-(20-methyl-2-(trifluoromethyl)-[1,10-biphenyl]-4-yl)-1,2,4-oxadiazol-3-yl)-benzyl)methyl)-amino)-propanoic acid, named as [¹⁸F]FS1P1. Tissue distribution of [¹⁸F]FS1P1 was performed using Sprague-Dawley rats and no in vivo defluorination was confirmed. PET brain studies of [¹⁸F]FS1P1 were also performed to compare with [¹¹C]CS1P1 in nonhuman primate, and showed that [¹⁸F]FS1P1 and [¹¹C]CS1P1 have almost identical pharmacokinetics in nonhuman primate brain. Radiometabolite analysis of nonhuman primate plasma samples indicated negligible formation of radiometabolites in vivo post-injection of [¹⁸F]FS1P1. Thus, [¹⁸F]FS1P1 can be used as a S1P1 F-18 radiotracer to investigate neuroinflammation.

### SUMMARY OF THE INVENTION

Among the various aspects of the present disclosure is the provision of a compositions for binding sphingosine-1-phosphate receptor 1 (S1P1), imaging of S1P1, and methods of use thereof.

Generally, the present invention relates to various compounds, compositions and methods that are useful for binding to, modulating or monitoring expression of sphingosine-1-phosphate (S1P) receptors in tissue. In various aspects, the present invention is directed to a compound having a structure of Formula (I) or (II), a pharmaceutically acceptable salt or a prodrug thereof:

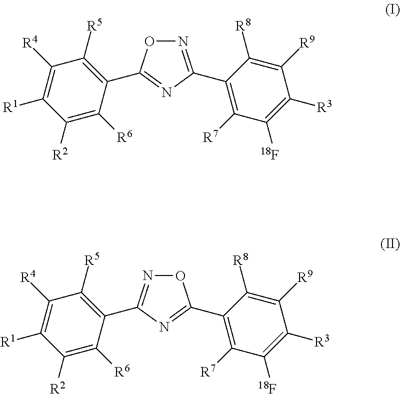

wherein: R¹ is substituted or unsubstituted aryl;
R² is C₁-C₄ haloalkyl or cyano;
R³ and R⁹ are each independently hydrogen, halo, hydroxy, substituted or unsubstituted amino, substituted or unsubstituted sulfonyl, substituted or unsubstituted hydrocarbyl;
R⁴ are each independently hydrogen, substituted or unsubstituted C₁-C₆ alkyl, or substituted or unsubstituted C₁-C₆ alkoxy; and
R⁵, R⁶, R⁷ and R⁸ are each independently hydrogen, halo, hydroxy, substituted or unsubstituted C₁-C₆ alkyl, or substituted or unsubstituted C₁-C₆ alkoxy.

In other aspects, the present invention is directed to compounds having a structure of Formula I or II that are radiolabeled with a radioactive isotope.

In further aspects, the present invention is directed to pharmaceutical compositions comprising a radiolabeled compound of Formula I or II, wherein the compound of Formula I or II comprises at least one synthetic radioactive isotope.

Additional aspects of the present invention include methods of diagnosing or monitoring an S1P1 associated disease, disorder or condition in a mammal comprising administering a composition comprising a radiolabeled compound of Formula I or II to the mammal and detecting the compound in the mammal.

In some aspects, the present invention is also directed to methods of quantifying S1P1 expression in a mammal comprising administering a composition comprising a radiolabeled compound of Formula I or II to the mammal and detecting the compound in the mammal.

In still further aspects, the present invention is directed to methods of treating an S1P1 associated disease, disorder, or condition in a subject in need thereof, comprising: administering to the subject a pharmaceutical composition comprising a therapeutically effective amount of an S1P1 modulating agent comprising any compound described herein, and inhibiting, slowing the progress of, or limiting the development of the S1P1 associated disease, disorder, or condition

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts prep-HPLC chromatogram of the reaction solution.

FIG. 2A depicts QC-HPLC chromatogram of final dose.

FIG. 2B depicts QC-HPLC chromatogram of final dose (co-injected with reference standard compound TZ3321.

FIG. 3 depicts quality control of [¹⁸F]FS1P1.

FIG. 4 depicts a typical analytical HPLC trace of formulated [¹⁸F]FS1P1. The top panel shows UV trace for [¹⁸F]FS1P1 with 10% ethanol in 0.9% saline. The second panel from the top shows the [¹⁸F]FS1P1 radiochemical trace. The second panel from the bottom shows the UV trace for co-injection of [¹⁸F]FS1P1 and CS1P1. The bottom panel shows the radiochemical trace for co-injection of [¹⁸F]FS1P1 and CS1P1. Analytical HPLC conditions: Phenomenex SB-C18, 250×4.6 mm, mobile phase 75% acetonitrile in 0.1 M ammonium formate, pH 4.5, flow rate 1.5 mL/min, detection wavelength 254 nm.

FIG. 5 depicts a typical analytical HPLC trace of [¹⁸F]-intermediates. The top panel shows radiochemical trace for [¹⁸F]12 (step 1); Analytical HPLC conditions: Agilent SB-C18 column, 250×4.6 mm, mobile phase 90% acetonitrile in 0.1 M ammonium formate, pH 4.5, flow rate 1.5 mL/min. The middle panel shows a [¹⁸F]imine radiochemical trace (step 2). The bottom panel shows a radiochemical trace of [¹⁸F]amino acid (step 3), Analytical HPLC conditions: Phenomenex SB-C18, 250×4.6 mm, mobile phase 75% acetonitrile in 0.1 M ammonium formate, pH 4.5, flow rate 1.5 mL/min.

FIG. 6 depicts in vitro autoradiography of rat spinal cord and brain sections by [¹⁸F]FS1P1 and blocked by CS1P1 (5 μM).

FIG. 7A depicts tissue distribution of [¹⁸F]FS1P1 in normal SD rats. The uptake (ID %/gram) of [¹⁸F]FS1P1 was high in the tissues including heart, lung, pancreases, spleen, kidney, liver, brain, small intestine, and large intestine;

FIG. 7B depicts brain tissue distribution of [¹⁸F]FS1P1 in normal SD rats. The uptake (ID %/gram) of [¹⁸F]FS1P1 in brain regionals of interest was relative high ranging from 0.4 to 0.8. Data represents mean±SEM.

FIG. 8A depicts time-activity curves of microPET studies of [¹¹C]CS1P1 and [¹⁸F]FS1P1 in brain of non-human primates (macaques). This was done using a Focus 220 microPET scanner to perform 0˜2 hr dynamic scan for data acquisition from the brains of male macaques (9-10 kg) post injection of ˜0.35 GBq of radiotracer via the venous catheter. The total brain uptakes of [¹⁸F]FS1P1 and [¹¹C]CS1P1 were very similar;

FIG. 8B depicts the uptake of [¹⁸F]FS1P1 in different regions of the macaque brain.

FIG. 8C depicts representative PET images and co-registered MRI images of [¹⁸F]FS1P1 in macaque brain.

FIG. 8D depicts the total brain uptakes (SUV) of [¹⁸F]FS1P1 using different doses, which were also identical.

FIGS. 9A and 9B depict radiometabolite analysis of nonhuman primate plasma sample post-injection of [18F]FS1P1. No major metabolite was detected in macaque plasma samples collected at 5, 15, 30, and 60 min post injection.

FIG. 10A depicts PET scans of male (88 Kg), dose of 7.2 mCi of [¹¹C]CS1P1, 28 years old, SIEMENS (patient ID: 1179101).

FIG. 10B depicts PET scans of the brain of the subject in FIG. 10A.

FIG. 10C depicts activity in different body tissues over time for the subject in FIG. 10A.

FIG. 11A depicts tracer uptake over time for [¹⁸F]-TZ4877 in rats.

FIG. 11B depicts [¹⁸F]-TZ4877 radiometabolite analysis in rat plasma.

FIG. 11C depicts [¹⁸F]-TZ4877 radiometabolite analysis in rat brain.

FIG. 12A depicts representative sagittal (left) and coronal (right) microPET images of [¹⁸F]TZ4877 in whole mice bodies of S. aureus infected and sham mice.

FIG. 12B depicts S1P1 activity in S. aureus infected mice brain. Representative sagittal (top) and coronal (bottom) of infected and sham mice are shown.

FIG. 12C depicts a quantification of brain uptake of [¹⁸F]-TZ4877 in S. aureus infected and sham mice.

FIG. 12D depicts tissue uptake of [¹⁸F]TZ4877 in S. aureus infected versus sham mice.

FIG. 12E depicts IHC staining in S. aureus infected and sham mice.

FIG. 13A depicts [¹⁸F]TZ4877 uptake in various tissues of sham and S. aureus infected mice with and without 5 mg/kg NBR0213, a specific S1P1 inhibitor.

FIG. 13A depicts [¹⁸F]TZ4877 uptake in various tissues of sham and S. aureus infected mice with and without 20 μg/kg S1P1 DsiRNA.

FIG. 13C depicts IHC staining in S. aureus infected mice and infected mice with S1P1 DsiRNA treatment (20 μg/kg).

FIG. 14 depicts uptake of [¹⁸F]TZ4877 in various tissues of sham mice, infected mice, and infected mice also treated with 5 mg/kg Enrofloxacin administered 12 hours prior to inoculation and 24 hours prior to tracer injection.

FIG. 15A depicts presence of [¹⁸F]TZ4877, its major metabolite, and other compounds over time, including in blood, plasma, and PCIF.

FIG. 15B depicts uptake of [¹⁸F]TZ4877 in various brain regions over time.

FIG. 16A depicts in vitro saturation binding autoradiograph analysis of [³H]CS1P1 of human brain gray matter.

FIG. 16B depicts in vitro saturation binding autoradiograph analysis of [³H]CS1P1 of rat brain cortex.

FIG. 16C depicts in vitro saturation binding autoradiograph analysis of [³H]CS1P1 of rat brain hippocampus.

FIG. 16D depicts representative images of [³H]CS1P1 in human frontal cortex.

FIG. 16E depicts representative images of [³H]CS1P1 in rat brain.

FIG. 16F depicts a representative image of tritium ART-123 standard.

FIG. 17A depicts time-activity curves of microPET studies of [¹⁸F]TZ8247, [¹⁸F]TZ8248, and [¹⁸F]TZ823 compared to [¹⁸F]FS1P1 in brain of non-human primates.

FIG. 17B depicts non-human primate brain pharmacokinetics of eight F-18 radiotracers.

FIG. 17C depicts representative scans of eight F-18 radiotracers.

FIG. 18A is a graph of the total brain uptake (SUV) versus time for various F-18 radiotracer compounds.

FIG. 18B is also a graph of the total brain uptake (SUV) versus time for various F-18 radiotracer compounds.

FIG. 19A-D is a MicroPET study of [¹⁸F]TZ82112 in the macaque brain. (A) The standardized uptake of [¹⁸F] TZ82112 in the macaque brain, data represent mean±SD, n=2; (B) Representative PET and coregistrated MRI images of [¹⁸F] TZ82112 the macaque brain; (C) The uptake of [¹⁸F] TZ82112 in different regions of the macaque brain; (D) MicroPET studies of [¹⁸F] TZ82112 in the macaque brain with self-blocking and blocking with S1PR1 specific ligand ponesimod.

FIG. 19E is a whole-body ex vivo biodistribution study of [¹⁸F]TZ82112 in SD rats. All data are referred to as mean±SD, n=3.

### DETAILED DESCRIPTION OF THE INVENTION

The present invention relates to various compounds, compositions and methods that are useful for monitoring expression of sphingosine-1-phosphate (S1P) receptors in tissue. To this end, various compounds having a high affinity for S1P receptors are provided herein. These compounds can be, in some embodiments, labeled with a radiolabel (e.g., a radioisotope) and be used alongside molecular imaging techniques to visualize S1P expression in tissue (e.g., in a subject). Also provided are methods of monitoring S1P1 associated diseases, disorders, or conditions by monitoring S1P expression in a subject. The present invention further includes methods of tracking and/or monitoring the effectiveness of a certain therapy or treatment for an S1P1 associated disease, disorder, or condition. Still further, the present invention provides for methods of treating S1P1 associated diseases, disorders, or conditions by administering a compound having a high affinity for S1P1. Even further, the present invention provides for processes of preparing these compounds and compositions.

Sphingosine-1-phosphate receptor 1 (S1P1) is a reliable biomarker for assessing the inflammation response for in diseases. S1P1 play a key role in central nervous system (CNS), vascular and lymphatic system, immune system, and cancer. Preclinical results indicated that C-11 labeled [11C]CS1P1 is a promising radiotracer for in vivo quantifying S1 PR1 expression changes in different inflated models of animals. Currently clinical investigation of [11 C]CS1 P1 for the multiple sclerosis (MS) is under progress.

The longer half-life of F-18 (t1/2=109.8 min c.f. C-11 t1/2=20.4 min) provides fewer time constraints on production F-18 radiopharmaceutical and permit longer scan sessions with high target-to-reference ratios, facilitating radiopharmaceutical distribution and multiple clinical trials. Currently, no promising F-18 S1P1 radiotracer have been reported for clinical study. Because the structure of CS1P1 has a fluorine atom on the aromatic ring, exploration of the F-18 radiochemistry to make the F-18 labeled [18F]CS1P1 will provide a promise F-18 radiotracer for quantitatively measure of S1P1 expression in diseases.

Herein is described a procedure of making F-18 labeled [¹⁸F]CS1P1. This procedure also could be used for making other F-18 labeled molecules that have fluorine on the aromatic ring.

As noted, S1P receptor plays a key role for inflammatory disease. Since the radiotracer is able to quantify the change of the S1P receptor, it provides a noninvasive methodology to quantify the expression of S1P receptor in vivo response to the progression of inflammatory diseases. The radiotracers described herein can be used as a unique tool to assess the therapeutic response for treating inflammatory diseases using S1P inhibition. The compounds as described herein also can be therapeutic drugs for treating inflammation diseases. In both cases, the compounds and radiotracers can be used to monitor, diagnose and/or treat various neuroinflammatory diseases, pulmonary infection diseases, and vascular injury relative diseases that are associated with changes of S1P receptor levels.

### Compounds

Various compounds described herein include an S1P1 modulating agent. As defined herein, an S1P1 modulating agent is a compound that binds to and/or modulates S1P1 surface expression on a cell.

The S1P1 modulating agent can comprise a compound having the structure of Formula (I) or (II), as defined herein. In addition, the modulating agent can comprise a benzoxazole or an oxadiazole core.

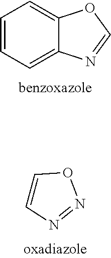 [SMILES: c1ccc2ocnc2c1 ; c1conn1]

Various compounds useful for targeting/modulating the S1P receptor, particularly S1P1, include compounds of Formula (I) or Formula (II), or a pharmaceutically acceptable salt or a prodrug thereof:

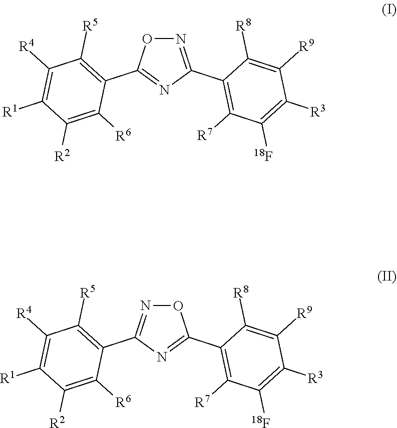

wherein:
R¹ is substituted or unsubstituted aryl (e.g., C₁-C₆ alkyl substituted phenyl);
R² is C₁-C₄ haloalkyl or cyano;
R³ and R⁹ are each independently hydrogen, halo, hydroxy, substituted or unsubstituted amino, substituted or unsubstituted sulfonyl, substituted or unsubstituted hydrocarbyl;
R⁴ are each independently hydrogen, substituted or unsubstituted C₁-C₆ alkyl, or substituted or unsubstituted C₁-C₆ alkoxy; and
R⁵, R⁶, R⁷ and R⁸ are each independently hydrogen, halo, hydroxy, substituted or unsubstituted C₁-C₆ alkyl, or substituted or unsubstituted C₁-C₆ alkoxy.

In various embodiments, R⁴ is substituted or unsubstituted C₁-C₆ alkoxy. In additional embodiments, R⁴ is a halo-substituted C₁-C₆ alkoxy. For example, R⁴ can be a C₁-C₆ fluoroalkoxy (e.g., —OCH₂CH₂F).

In some embodiments, R⁴ is hydrogen or a C₁-C₆ alkyl. In certain embodiments, R⁴ is hydrogen.

In various embodiments, R² is —CF₃.

In some embodiments, at least one of R³ and R⁹ is a substituted or unsubstituted C₁-C₆ alkyl or a substituted or unsubstituted C₁-C₆ alkoxy. For example, in various embodiments, at least one of R³ and R⁹ is —CH₂(OCH₂CH₂)ₙOH or —(OCH₂CH₂)ₙOH, where n is from 0 to 10, from 0 to 8, from 0 to 6, from 0 to 4, or from 0 to 2. In certain embodiments, n is from 1 to 10, from 1 to 8, from 1 to 6, from 1 to 4. For example, n can be from 1 to 3 or from 1 to 2.

In various embodiments, at least one of R³ and R⁹ is —CH₂R¹¹ where R¹¹ is substituted alkoxy, substituted or unsubstituted amino, a substituted or unsubstituted amido, an azide, a substituted or unsubstituted sulfonyl, a substituted sulfur-containing ring, or a substituted or unsubstituted nitrogen-containing ring

In various embodiments, at least one of R³ and R⁹ is

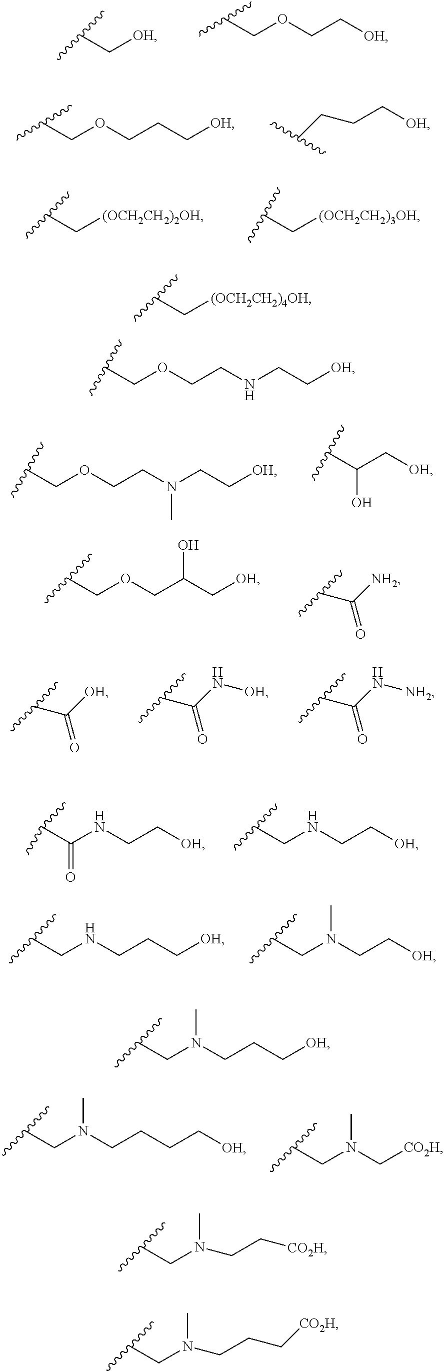

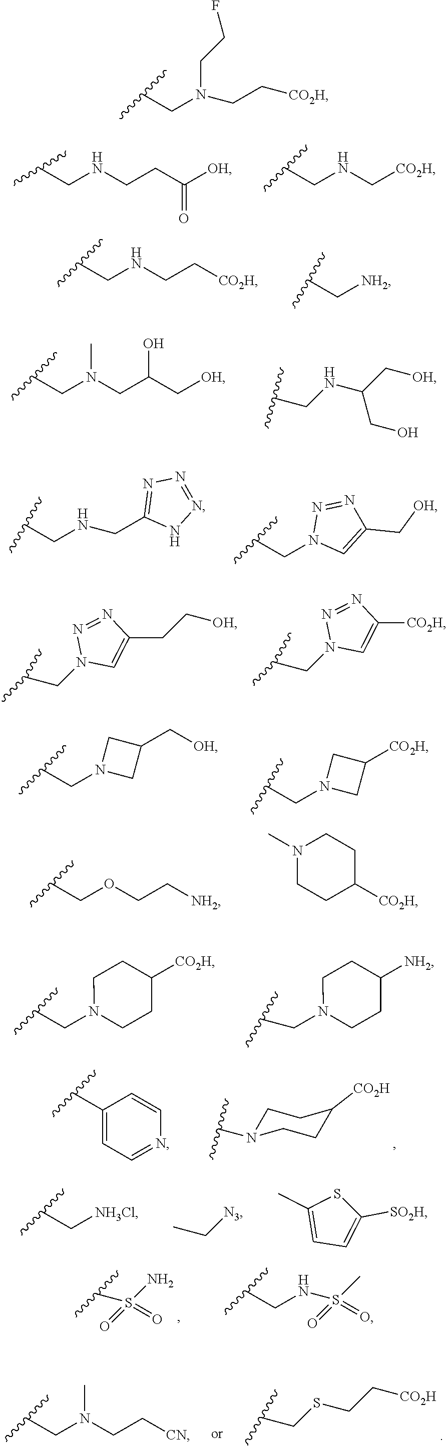

In some embodiments, at least one of R³ and R⁹ is —CH(OH)CH₂OH.

In certain embodiments, R³ is one of the aforementioned moieties. In these and other embodiments, R⁹ is hydrogen.

In various embodiments, R⁵, R⁶, R⁷, R⁸, and R⁹ are each independently hydrogen, halo, hydroxy, C₁-C₆ alkyl, or C₁-C₆ alkoxy. In some embodiments, R⁵, R⁶, R⁷, R⁸, R⁹, and R¹⁰ are each independently hydrogen or a C₁-C₆ alkyl. For example, R⁵, R⁶, R⁷, R⁸, and R⁹ can each independently be hydrogen.

As mentioned, the compounds of the present invention can have the structure of Formula I or Formula II. In some embodiments, the compound has the structure of Formula I or a pharmaceutically acceptable salt or a prodrug thereof. In other embodiments, the compound has the structure of Formula II or a pharmaceutically acceptable salt or a prodrug thereof.

In various embodiments, the compound can be selected from the group consisting of:

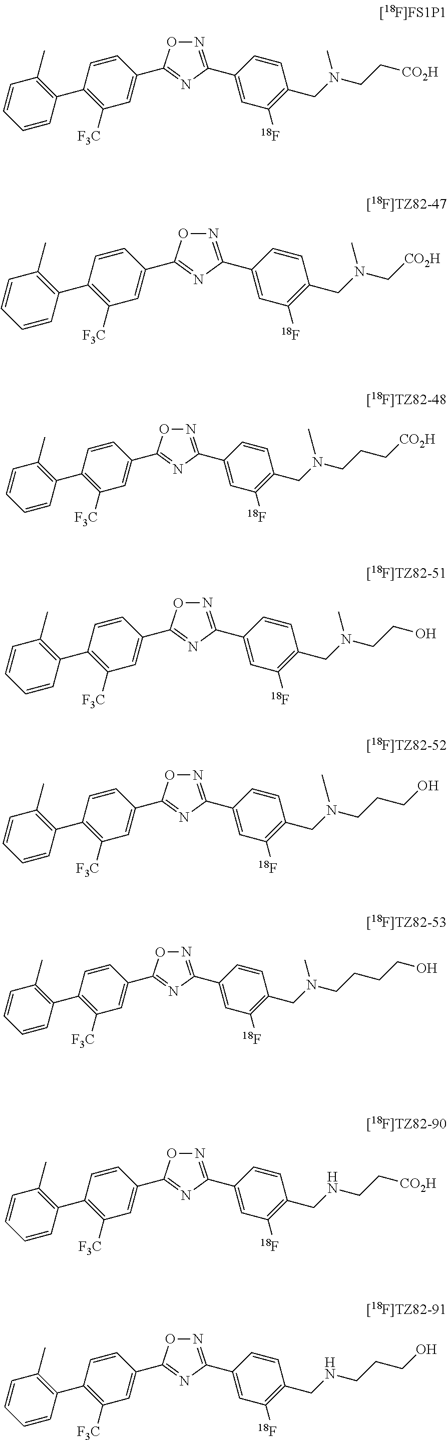

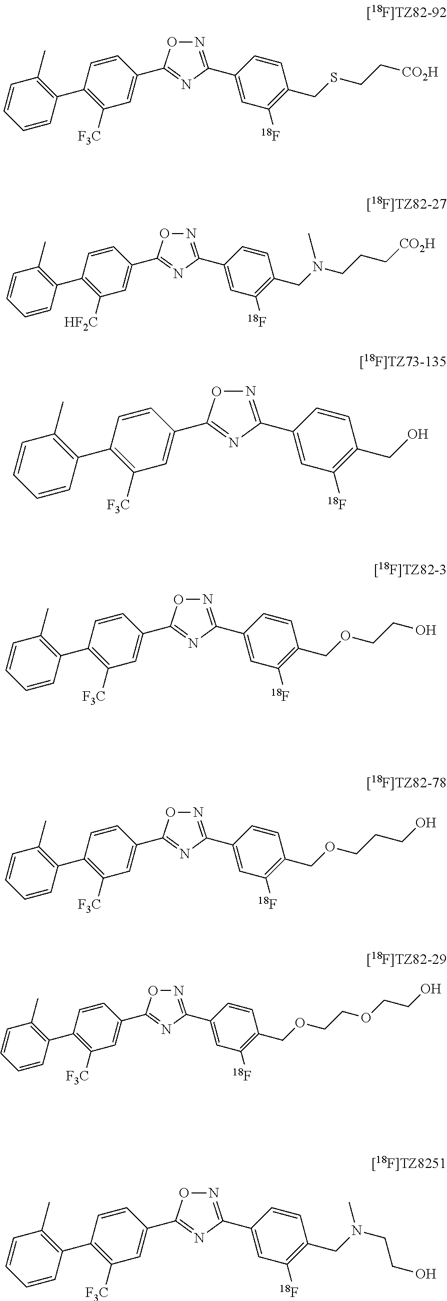

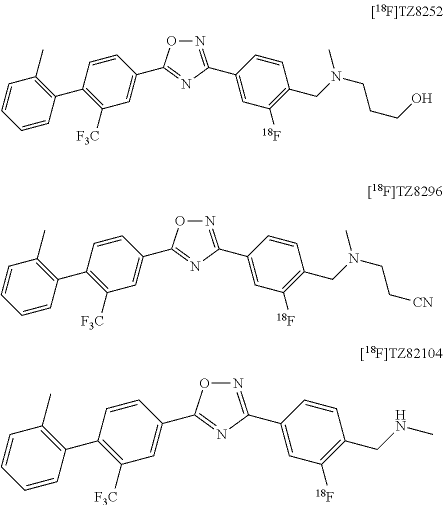 [SMILES: Cc1ccccc1-c1ccc(-c2nc(-c3ccc(CN(C)CCCO)c([18F])c3)no2)cc1C(F)(F)F ; Cc1ccccc1-c1ccc(-c2nc(-c3ccc(CN(C)CCC#N)c([18F])c3)no2)cc1C(F)(F)F ; CNCc1ccc(-c2noc(-c3ccc(-c4ccccc4C)c(C(F)(F)F)c3)n2)cc1[18F]]

pharmaceutically acceptable salts thereof. prodrugs thereof. and mixtures thereof.

Additionally, compounds useful for targeting/modulating the S1P receptor, particularly S1P1, include compounds of Formula (V), or a pharmaceutically acceptable salt or a prodrug thereof:

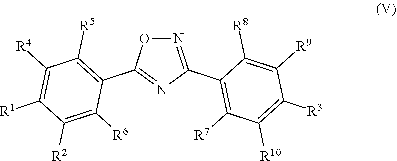

wherein:
R¹ is substituted or unsubstituted alkoxy;
R² is C₁-C₄ haloalkyl or cyano;
R³ and R⁹ are each independently hydrogen, halo, hydroxy, substituted or unsubstituted amino, substituted or unsubstituted sulfonyl, substituted or unsubstituted hydrocarbyl;
R⁴ are each independently hydrogen, substituted or unsubstituted C₁-C₆ alkyl, or substituted or unsubstituted C₁-C₆ alkoxy; and
R⁵, R⁶, R⁷, and R⁸ are each independently hydrogen, halo, hydroxy, substituted or unsubstituted C₁-C₆ alkyl, or substituted or unsubstituted C₁-C₆ alkoxy;
R¹⁰ is hydrogen or fluorine.

For compounds of Formula (V), preferably, R¹ is F¹⁸ substituted alkoxy and R², R³, R⁴, R⁵, R⁶, R⁷, R⁸, and R⁹ can be as described herein above for compounds of Formula (I) and (II). In some compounds, R¹⁰ is hydrogen and in other compounds R¹⁰ is fluorine.

Additionally, the compounds of Formula (III) can have R¹ be F¹⁸ substituted alkoxyl, where one or more of the hydrogen atoms of the alkoxy group are replaced with deuterium.

Compounds of formula (V) can be

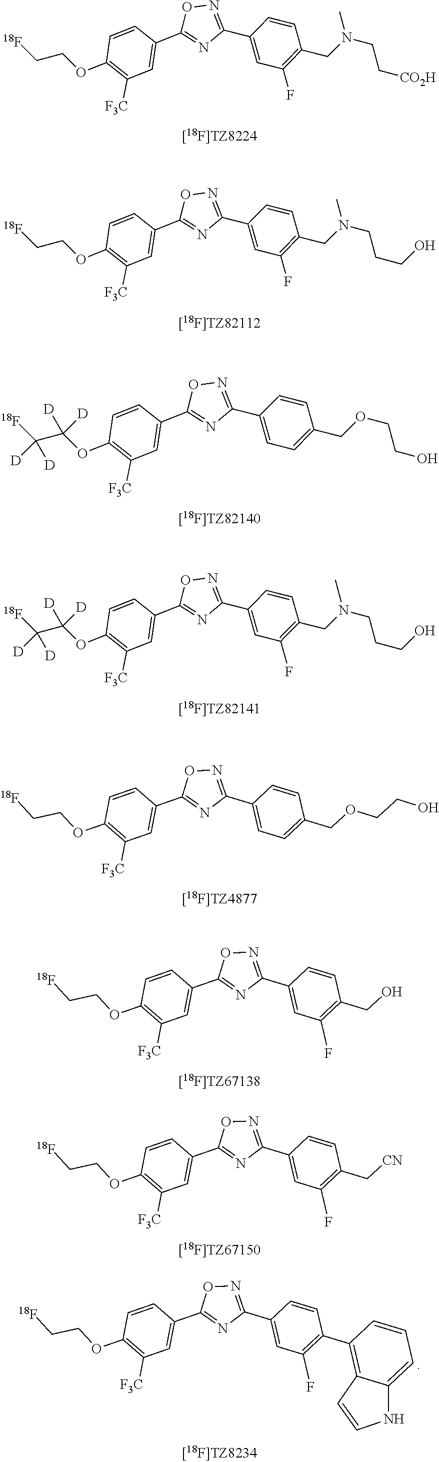

In various cases the compound (e.g., S1P1 modulating agent) has a high binding affinity and selectivity for the S1P1 over other S1P receptors (e.g., S1P2-S1P5). In some cases, the compound (e.g., S1P1 modulating agent) binds to the receptor with high affinity and triggers internalization of the receptor into a cell, thereby reducing S1P1 surface expression on the cell. As will be described herein, this reduction in S1P1 surface expression can be useful in the treatment of various S1P1 associated diseases, disorders, or conditions.

Methods of determining the affinity of a compound for its receptor are generally known in the art. One way to measure affinity is use of a general competition binding assay. Descriptions of these binding assays, including methods of measuring the affinity of a compound for a S1P receptor are available in the art, for example in Rosenberg et al., (2015) “A practical process for the preparation of [(32)P]S1P and binding assay for S1P receptor ligands” Applied Radiation and Isotopes: Including Data, Instrumentation and Methods for use in Agriculture, Industry and Medicine. 102:5-9, which is incorporated herein by reference.

In some embodiments, the compounds (e.g., modulating agents) of the present invention compete with S1P binding to a S1P receptor with an IC₅₀ of less than 100 nM, less than 75 nM, less than 50 nM, less than 25 nM, or less than 15 nM. For example, the compounds can have an IC₅₀ of from about 1 nM to about 15 nM, from about 1 nM to about 10 nM, from about 1 nM to about 5 nM or from about 5 nM to about 10 nM. One advantage of this invention is the higher affinity some of the compounds have for the S1P1 receptor over the other four subtypes. In some embodiments, the compounds (e.g., modulating agents) of the present invention have a high affinity (e.g., less than 100 nM, less than 75 nM, less than 50 nM, less than 15 nM) for the S1P1 receptor while having a lower affinity (e.g., >1000 nM) at the other S1P receptors (S1P2-S1P5).

The present disclosure provides for a radiolabeled compound or composition, or a compound or composition with a radionuclide.

The radiolabeled compound or composition can comprise any compound (e.g., modulating agent) described herein (e.g., a compound of Formula I or II) radiolabeled with a radioactive isotope. References herein to “radiolabeled” include a compound where one or more atoms are replaced or substituted by an atom having an atomic mass or mass number different from the atomic mass or mass number typically found in nature (i.e., naturally occurring). One non-limiting exception is ¹⁸F, which allows detection of a molecule which contains this element without enrichment to a higher degree than what is naturally occurring. Compounds carrying the substituent ¹⁸F may thus also be referred to as “labeled” or the like. The term radiolabeled may be interchangeably used with “isotopically-labeled”, “labeled”, “isotopic tracer group”, “isotopic marker”, “isotopic label”, “detectable isotope”, “radiotracer, and “radioligand”.

In one embodiment, the compound comprises a single radiolabeled group (i.e., ¹⁸F).

S1P1 modulates lymphocyte trafficking, a hallmark of inflammation. Up-regulated S1P1 levels can be detected in: multiple sclerosis (ms), cancer, cardiovascular disease, or other inflammatory diseases. Tracking S1P1 expression in vivo can assist in assessing therapeutic efficacy or assessing disease progression.

Compounds of the present invention can be synthesized as described in Examples 1-3 and 10-11. The processes include reacting a [¹⁸F] fluoride salt with a precursor compound of formula (III) or (IV)

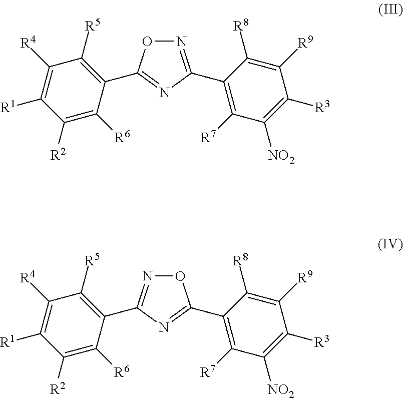

to form a [¹⁸F] radiolabeled precursor (NO₂ is substituted for the ¹⁸F label). The [¹⁸F] radiolabeled precursor can be further functionalized as shown in Examples 1-3 and 10-11.

### Pharmaceutical Compositions or Formulations

As noted, various embodiments of the present invention relate to pharmaceutical compositions comprising a therapeutically effective amount of at least one of the compounds as described herein (e.g., a compound of Formula (I) or Formula (II) or salt or prodrug thereof). In various embodiments, the pharmaceutical composition comprises at least one radiolabeled compound of Formula (I) or (II) as described herein.

The pharmaceutical composition can comprise from about 0.001 mg to about 10 g of the radiolabeled compound and at least about 10 wt. %, at least about 25 wt. %, at least about 50 wt. %, at least about 75 wt. %, at least about 90 wt. %, or at least about 95 wt. % of the compound in the pharmaceutical composition is radiolabeled.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers. Pharmaceutically acceptable excipients for use in the compositions of the present invention are selected based upon a number of factors including the particular compound used, and its concentration, stability and intended bioavailability; the subject, its age, size and general condition; and the route of administration.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. Routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. For example, the agents of use with the current disclosure can be formulated by known methods for administration to a subject using several routes including: parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

The pharmaceutical compositions can be formulated, for example, for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. Pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated to delay disintegration and absorption.

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

Compound described herein can be prepared as a salt. “Salt” as used herein refers to pharmaceutically acceptable salts of the compounds described herein which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). Examples of pharmaceutically acceptable salts include, but are not limited to, nontoxic acid addition salts which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

In other embodiments, the compounds may be prepared as “prodrugs” in a pharmaceutically acceptable composition/formulation. As used herein, the term “prodrug” refers to a derivative of a compound that can hydrolyze, oxidize, or otherwise react under biological conditions (in vitro or in vivo) to provide a compound as described herein. Prodrugs may only become active upon some reaction under biological conditions, but they may have activity in their unreacted forms. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs and their uses are well known in the art (see, e.g., Berge, et al. 1977 J. Pharm. Sci. 66:1-19). Prodrugs can typically be prepared using well-known methods, such as those described in Burger's Medicinal Chemistry and Drug Discovery (1995, Manfred E. Wolff ed., 5thed. 172-178, 931-932).

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Methods of Use

Dysregulation in S1P1 signaling is associated with inflammatory diseases in multiple organ systems, including the central nervous system (Soliven B et al., The neurobiology of sphingosine-1-phosphate signaling and sphingosine 1-phosphate receptor modulators. Neurology. February 2011; 76(8): S9-S14). S1P1 is extensively expressed on lymphocytes and endothelial cells, and it participates in neuroinflammatory process by regulating immune cell trafficking in the brain (Blaho V A et al., An update on the biology of sphingosine-1-phosphate receptors. Journal of lipid research. January 2014; 55(8):1596-1608). In the CNS, S1P1 is expressed in neurons and glia, including astrocytes, which modulate inflammatory responses throughout the gray and white matter; microglia, the specialized macrophages of the brain; and oligodendrocytes, which produce the myelin needed for nerve conduction (Soliven B et al., 2011; Nishimura H et al., Cellular Localization of Sphingosine-1-phosphate Receptor 1 Expression in the Human Central Nervous System. J Histochem Cytochem. September 2010; 58(9):847-856). The relevance of S1P1 in clinical disease has become readily apparent with the FDA approval of the S1P1 modulator FTY720 (fingolimod) for treating relapsing-remitting multiple sclerosis (RR-MS), which is a chronic autoimmune, inflammatory, demyelinating neurodegenerative disease (Dev K K et al., Brain sphingosine-1-phosphate receptors: Implication for FTY720 in the treatment of multiple sclerosis. Pharmacol Therapeut. January 2008; 117(1):77-93).

The role of S1P/S1P1 in vascular inflammation has also been studied in stroke-prone spontaneously hypertensive rats (SHRSPs). S1P stimulates inflammatory signaling pathways via transactivation of receptor tyrosin kinase (RTK) through S1P1, leading to increased expression of intercellular adhesion molecular 1 (ICAM-1) and vascular cell adhesion protein 1 (VCAM-1) and promotes monocyte adhesion (Yogi A et al., (2011) Sphingosine-1-Phosphate-Induced Inflammation Involves Receptor Tyrosine Kinase Transactivation in Vascular Cells Upregulation in Hypertension. Hypertension 57:809-818). Moreover, high S1P1 expression has been found in endothelial cells, macrophages and proliferated vascular smooth muscle cells (VSMCs), which are major components of atherosclerotic plaques (Daum G. et al., (2009) Sphingosine 1-phosphate: a regulator of arterial lesions. Arterioscler Thromb Vasc Biol 29:1439-1443). This is in agreement with a study that demonstrated that a specific S1P1 agonist treatment reduced lesion size in low-density lipoprotein receptor (LDLR)-deficient mice (Poti F et al., (2013) KRP-203, Sphingosine 1-Phosphate Receptor Type 1 Agonist, Ameliorates Atherosclerosis in LDL-R−/− Mice. Arterioscl Throm Vas 33:1505-1512). Therefore, S1P1 is a promising target for molecular imaging of atherosclerotic lesions, and may serve as a potential therapeutic target to inhibit atheroprogression and plaque vulnerability.

### Methods of Quantifying S1P1 Expression In Vivo

Methods of quantifying S1P1 expression in vivo are provided. These methods comprise administering to a subject a composition comprising a radiolabeled compound as described herein and detecting the compound in the subject. In various embodiments, the radiolabeled compound has a high affinity (e.g., has an IC₅₀ less than 100 nM, less than 50 nM, or less than 25 nM) for the S1P1 receptor. In some embodiments, detecting the compound can comprise positron emission tomography (PET) imaging, and single photon emission computed tomography (SPECT) imaging, mass spectrometry, gamma imaging, magnetic resonance imaging (MRI), magnetic resonance spectroscopy, fluorescence spectroscopy, CT, ultrasound, or X-ray. In some embodiments, the molecular imaging technique is PET.

In various embodiments, the method comprises detecting the compound in a specific organ or organ system in the subject, in order to quantify the amount of S1P1-expression in the organ or organ system. In some embodiments, the method comprises quantifying S1P1 expression in a mammalian brain or central nervous system. In these cases, the subject's brain or central nervous system is imaged by, for example, positron emission tomography. Also envisioned are methods of quantifying S1P1 expression in other physiological organ systems (e.g., the cardiovascular system), or pathological organ states (e.g., cancerous tumors). In each case, the radiolabeled compound can be used to visualize S1P1 expression in the organ or organ system of interest using positron emission tomography or other suitable molecular imaging technique.

### Methods of Monitoring an S1P1 Associated Disease, Disorder or Condition

Also provided are methods of monitoring an S1P1 associated disease, disorder, or condition. The methods comprise administering a composition comprising a radiolabeled compound described herein to a subject in need thereof, and detecting the compound. The compound can be detected using any suitable molecular imaging technique. For example, the compound can be detected using positron emission tomography (PET) imaging, and single photon emission computed tomography (SPECT) imaging, mass spectrometry, gamma imaging, magnetic resonance imaging (MRI), magnetic resonance spectroscopy, fluorescence spectroscopy, CT, ultrasound, or X-ray. In some embodiments, the molecular imaging technique is PET.

Methods described herein are generally performed on a subject in need thereof. A subject in need of diagnosis described herein can be a subject suspected of having or at risk for developing an S1P1 associated disease, disorder, or condition. The subject in need of monitoring described herein can be a subject having, or diagnosed with the S1P1 associated disease disorder or condition. The subject in need of monitoring can be administered treatment for the S1P1 associated disease disorder or condition, prior to, concurrently with, or after the monitoring. A determination of the need for monitoring or diagnosis will typically be assessed by a history and physical exam consistent with the disease or condition at issue. The subject can be an animal subject, including a mammal, such as horses, cows, dogs, cats, sheep, pigs, mice, rats, monkeys, hamsters, guinea pigs, and chickens, and humans. For example, the subject can be a human subject.

The S1P1 associated disease, disorder or condition can be an inflammatory disease, a neuroinflammatory disease, a pulmonary infection disease, vascular injury disease, an autoimmune disease, a neurological disease, a psychological disorder, a cardiovascular disease, atherosclerosis, multiple sclerosis, rheumatoid arthritis, or cancer.

The radiolabeled compound can be detected in any organ or organ system in the subject as determined by one skilled in the art. For instance, when monitoring or diagnosing a neurological disease, using the methods described herein, the radiolabeled compound can be detected in the brain or nervous system.

### Methods of Treating an S1P1 Associated Disease, Disorder or Condition

Also provided is a process of treating an S1P1 associated disease, disorder, or condition in a subject in need administration of a therapeutically effective amount of a compound as described herein (e.g., a S1P1 modulating agent), so as to substantially inhibit an S1P1 associated disease, disorder, or condition, slow the progress of an S1P1 associated disease, disorder, or condition, or limit the development of an S1P1 associated disease, disorder, or condition. The method of treating an S1P1 associated disease disorder, or condition in a subject in need thereof comprises administering a pharmaceutical composition comprising a compound as described herein (e.g., a S1P1 modulating agent) and inhibiting, slowing the progress of, or limiting the development of the S1P1 associated disease, disorder, or condition.

In various embodiments, the compound as described herein (e.g., a S1P1 modulating agent) has a high affinity and selectivity for the S1P1. In some embodiments the compound having a high affinity and selectivity for S1P1 has an IC₅₀ for the S1P1 receptor of less than 100 nM, less than 50 nM, or less than 25 nM, and has an IC₅₀ for S1P2-S1P5 of greater than 1000 nM.

In some embodiments, the S1P1 associated disease, disorder, or condition is an inflammatory or autoimmune disease. For example, the S1P1 associated disease, disorder, or condition can be multiple sclerosis.

Methods described herein are generally performed on a subject in need thereof. A subject in need of the therapeutic methods described herein can be a subject having, diagnosed with, suspected of having, or at risk for developing an S1P1 associated disease, disorder, or condition. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of the compound (e.g., a S1P1 modulating agent) is, for example, that amount that would cause the desired therapeutic effect in a subject while minimizing undesired side effects. In various embodiments, an effective amount of a compound as described herein (e.g., a S1P1 modulating agent) can substantially inhibit an S1P1 associated disease, disorder, or condition, slow the progress of an S1P1 associated disease, disorder, or condition, or limit the development of an S1P1 associated disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a compound as described herein (e.g., a S1P1 modulating agent) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit an S1P1 associated disease, disorder, or condition, slow the progress of an S1P1 associated disease, disorder, or condition, or limit the development of an S1P1 associated disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a compound as described herein (e.g., a S1P1 modulating agent) can occur as a single event or over a time course of treatment. For example, a compound as described herein (e.g., a S1P1 modulating agent) can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an S1P1 associated disease, disorder, or condition.

A compound as described herein (e.g., a S1P1 modulating agent) can be administered simultaneously or sequentially with another agent, such as an antibiotic, an anti-inflammatory, or another agent. For example, a compound as described herein (e.g., a S1P1 modulating agent) can be administered simultaneously with another agent, such as an antibiotic or an anti-inflammatory. Simultaneous administration can occur through administration of separate compositions, each containing one or more of a compound as described herein (e.g., a S1P1 modulating agent), an antibiotic, an anti-inflammatory, or another agent. Simultaneous administration can occur through administration of one composition containing two or more of a compound as described herein (e.g., a S1P1 modulating agent), an antibiotic, an anti-inflammatory, or another agent. A compound as described herein (e.g., a S1P1 modulating agent) can be administered sequentially with an antibiotic, an anti-inflammatory, or another agent. For example, a compound as described herein (e.g., a S1P1 modulating agent) can be administered before or after administration of an antibiotic, an anti-inflammatory, or another agent.

### Methods of Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Bancyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present invention.

### Example 1: Radiosynthesis of [¹⁸F]TZ33-21 ([¹⁸F]FS1P1)

The radiosynthesis of [¹⁸F]FS1P1 was completed with a facile five-step procedure, followed by purification with semi-preparative HPLC as described below. The radiosynthesis of [¹⁸F]FS1P1 was accomplished with good radiochemical yield (15˜20%), high radiochemical purity (>99%), and high molar activity (>40 GBq/μmol, EOB).

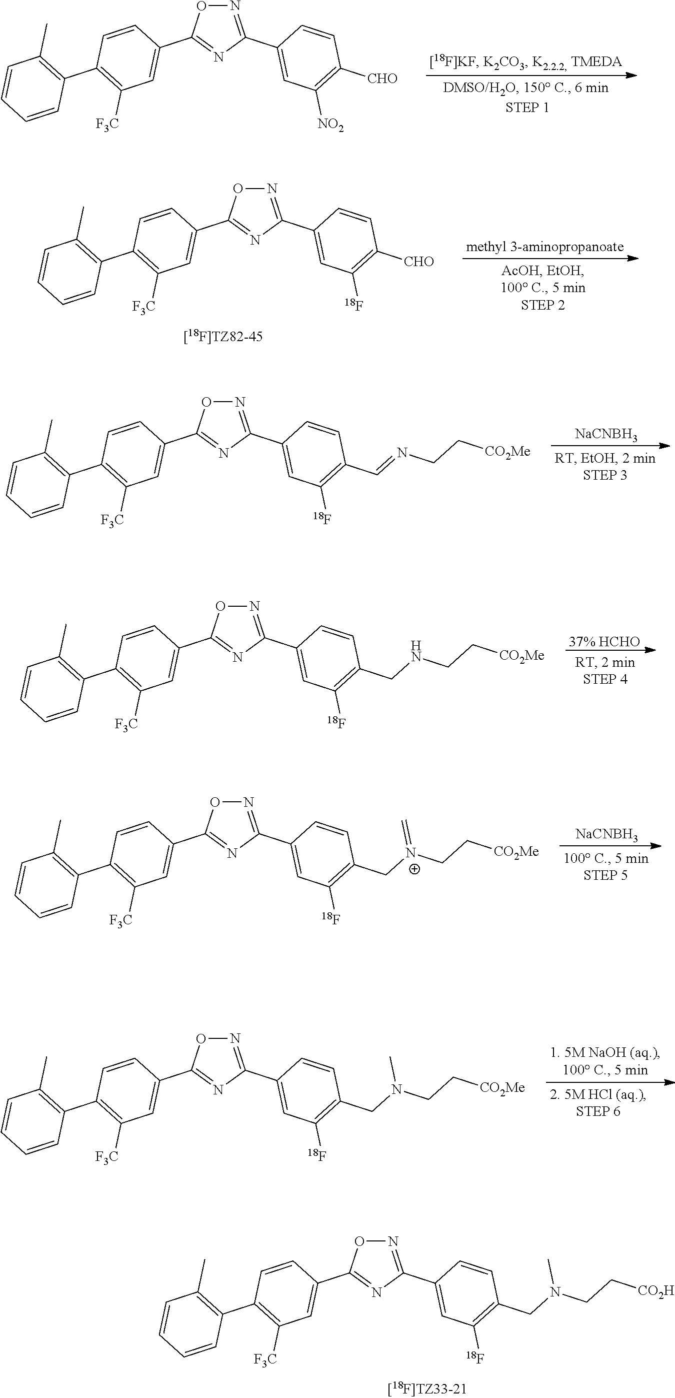

Procedures:

STEP 1: [¹⁸F]KF (˜7.4 GBq) aqueous was added to a vial containing 4,7,13,16,21,24-hexaoxa-1,10-diazabicyclo[8.8.8]-hexacosane (K₂₂₂) (6˜7 mg), and dried by azeotropic evaporation with acetonitrile (3×1 mL) under N₂ flow at 100° C. To the reaction vial was added precursor (4-5 mg) and TMEDA (30 μL) in anhydrous DMSO (300 μL) and H₂O (1 μL). The vessel was capped, vortexed, and then heated at 150° C. for 6 min.

STEP 2: Methyl 3-aminopropanoate hydrochloride (4.0 mg), TMEDA (4.5 μL), and CH₃CO₂H (5 μL) in anhydrous EtOH (300 μL) solution and were added to the reaction mixture from STEP 1 and the reaction mixture was heated at 100° C. for 5 min.

STEP 3: Upon cooling to room temperature, NaCNBH₃ (3 mg) was added to the reaction mixture. The vial was capped, shaken, and allowed to stand at room temperature for 2 min.

STEP 4: Formalin (50 μL) was added to the reaction mixture from STEP 3. The vial was capped, shaken occasionally and stand at room temperature for 2 min.

STEP 5: NaCNBH₃ (3 mg) was added to the reaction mixture. The vial was capped, shaken. Then the reaction mixture was heated at 100° C. for 5 min.

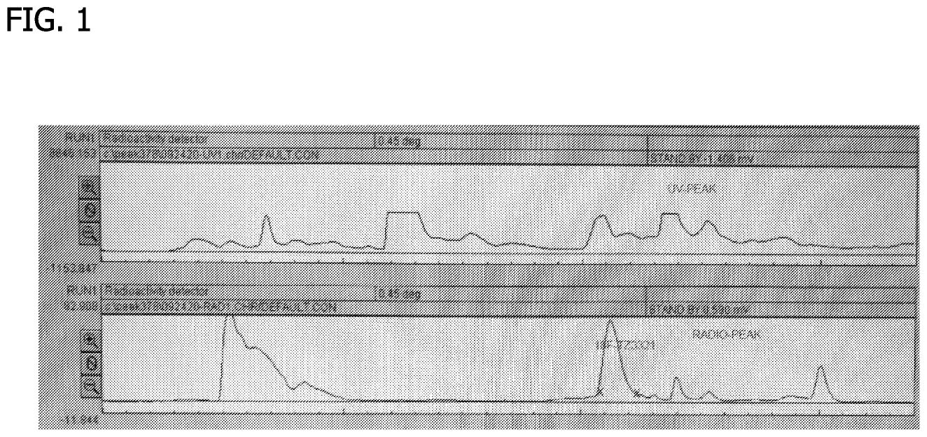

STEP 6: Upon cooling to room temperature, 5 M NaOH (200 μL) was added to the reaction mixture. The vial was capped, shaken, and then heated at 100° C. for 5 min. After cooling by water-bath, the reaction mixture was quenched by 200 μL 5 M HCl and 2.8 mL HPLC mobile phase (51% acetonitrile in 0.1 M ammonium formate buffer, pH 4.5). The solution was loaded onto a semi-preparative HPLC system for purification. The HPLC system contains a 5 mL injection loop, an Agilent SB-C18 column (250×9.4 mm, 5μ), a UV detector at 254 nm, and a radioactivity detector. With acetonitrile/0.1 M ammonium formate buffer (51/49, v/v, pH 4.5) as the eluent and at a flow rate of 4 mL/min, the retention time of the product was 25-28 min. The product collection was diluted using sterile water (˜50 mL) and then passed through a C18 Sep-Pak Plus cartridge. (See FIG. 1.) The trapped product was eluted using ethanol (0.6 mL), followed by 0.9% saline (5.4 mL). After sterile filtration into a glass vial, [¹⁸F]TZ33-21 was ready for quality control (QC) analysis and animal studies.

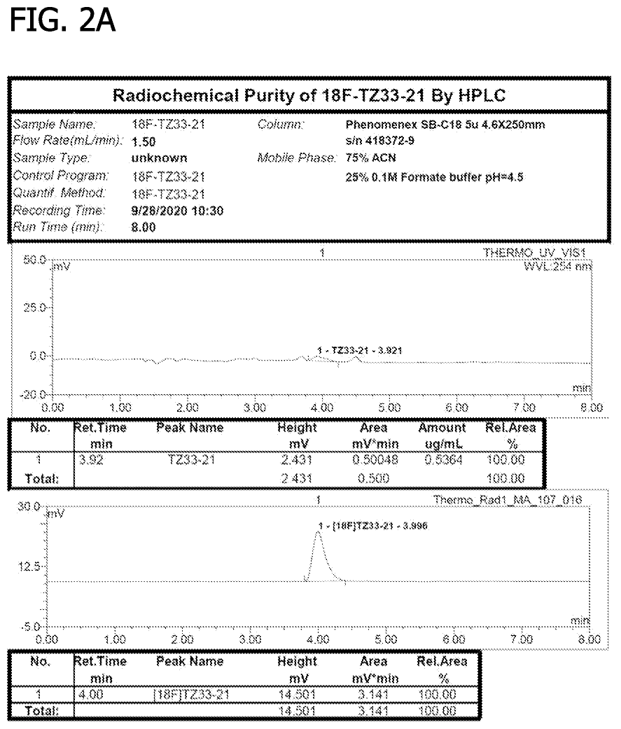

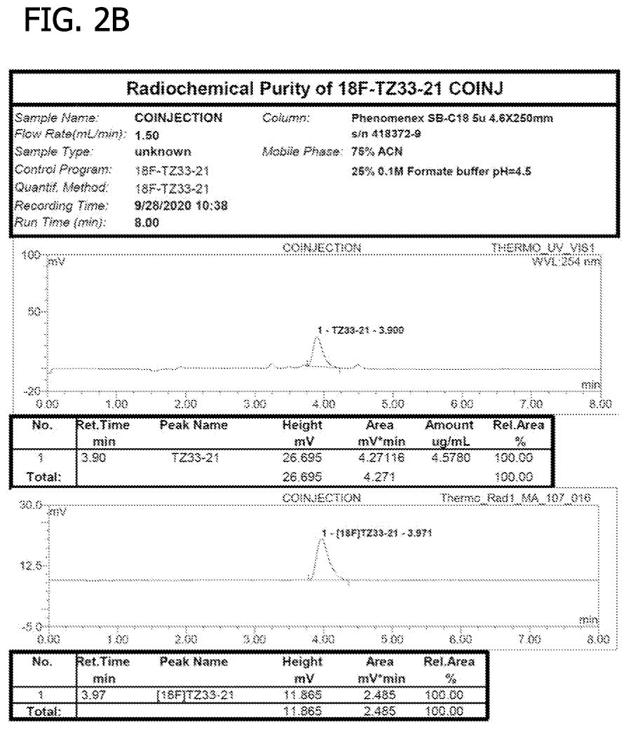

QC Analysis: To check the quality of [¹⁸F]TZ33-21, an aliquot of the sample was co-injected with the non-radiolabeled standard TZ33-21 sample solution onto an analytical HPLC system equipped with an Agilent Zorbax SB-C18 column (250×4.6 mm, 5μ) and UV absorbance at 254 nm; the mobile phase consisted of acetonitrile/0.1 M ammonium formate buffer (75/25, v/v, pH 4.5). Under these conditions, the retention time of [¹⁸F]TZ33-21 was 3.8 min at a flow rate of 1.5 mL/min. (See FIG. 2A and FIG. 2B.)

The radiochemical purity was >98%, the radiochemical yield for the four-step labeling was 10˜20% (decay corrected to the end of synthesis) and the specific activity was 100-274 GBq/μmol (decay corrected to the end of synthesis). The synthesis of [¹⁸F]TZ33-21 took about 120 min including the [¹⁸F]fluorine drying step.

### Example 2: Another Radiosynthesis of [¹⁸F]TZ33-21 ([18F]FS1P1)

An alternative synthesis is shown below.

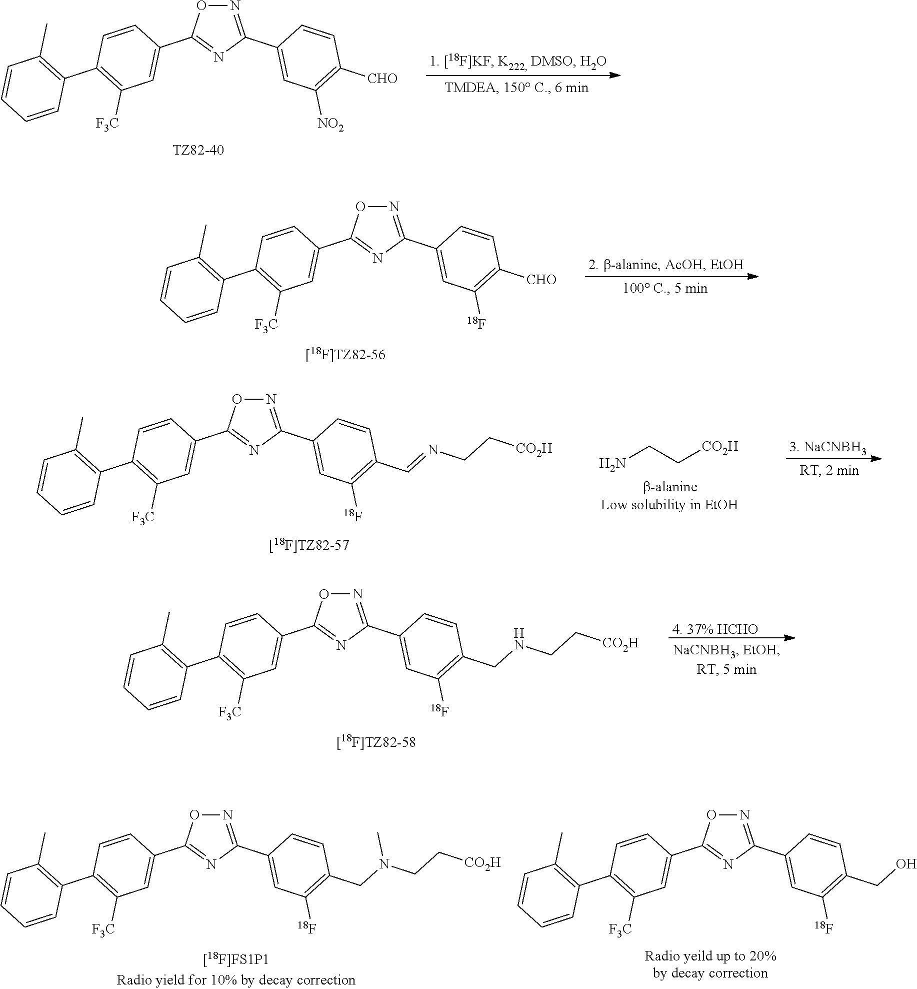

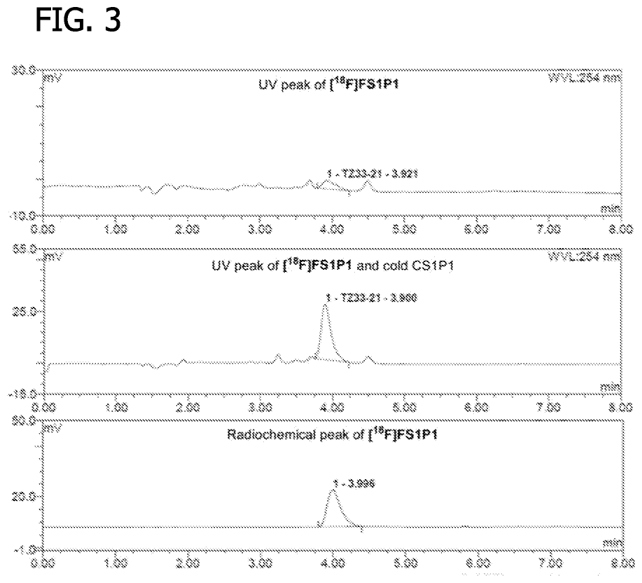

Another example synthesis is shown below. Quality control of [¹⁸F]FS1P1 is also presented (See FIG. 3). A multiple step F-18 chemistry method was developed and showed very good reproducibility.

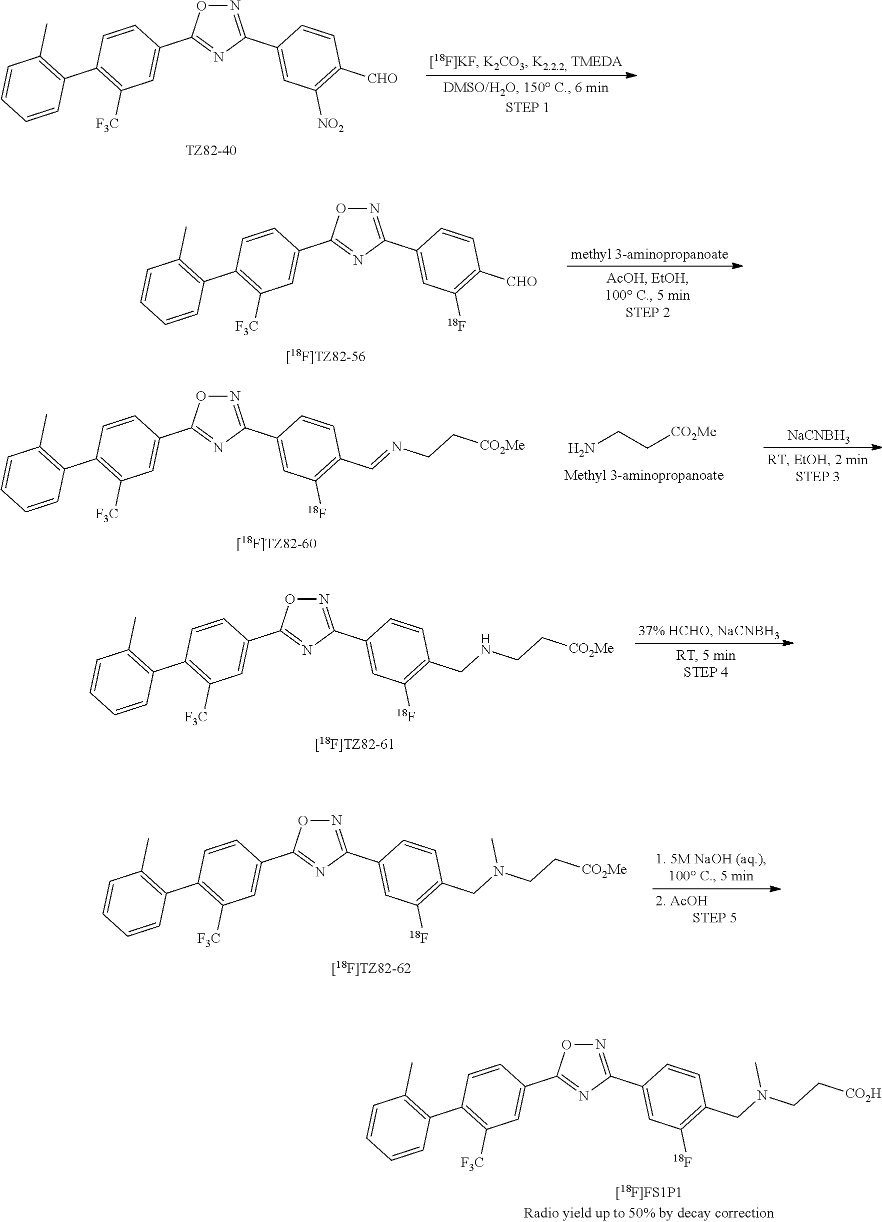

[¹⁶F]FS1P1 was radiosynthesized with good radio yield (up to 50% by decay correction), chemical and radiochemical purity (>99%), and high molar activity (>40 GBq/μmol, EOB).

### Example 3: A Further Radiosynthesis of [¹⁸F]TZ33-21 ([18F]FS1P1)

To radiosynthesize [¹⁸F]FS1P1, it was started with direct nucleophilic radiofluorination of the nitroarenes including uncycled precursor 5 or the cycled precursor 6, and then removed the t-butyl protection group using trifluoroacetic acid (TFA). Therefore, corresponding uncycled nitro precursor 5, and the cycled precursor 6 were prepared as depicted in Scheme 1, below.

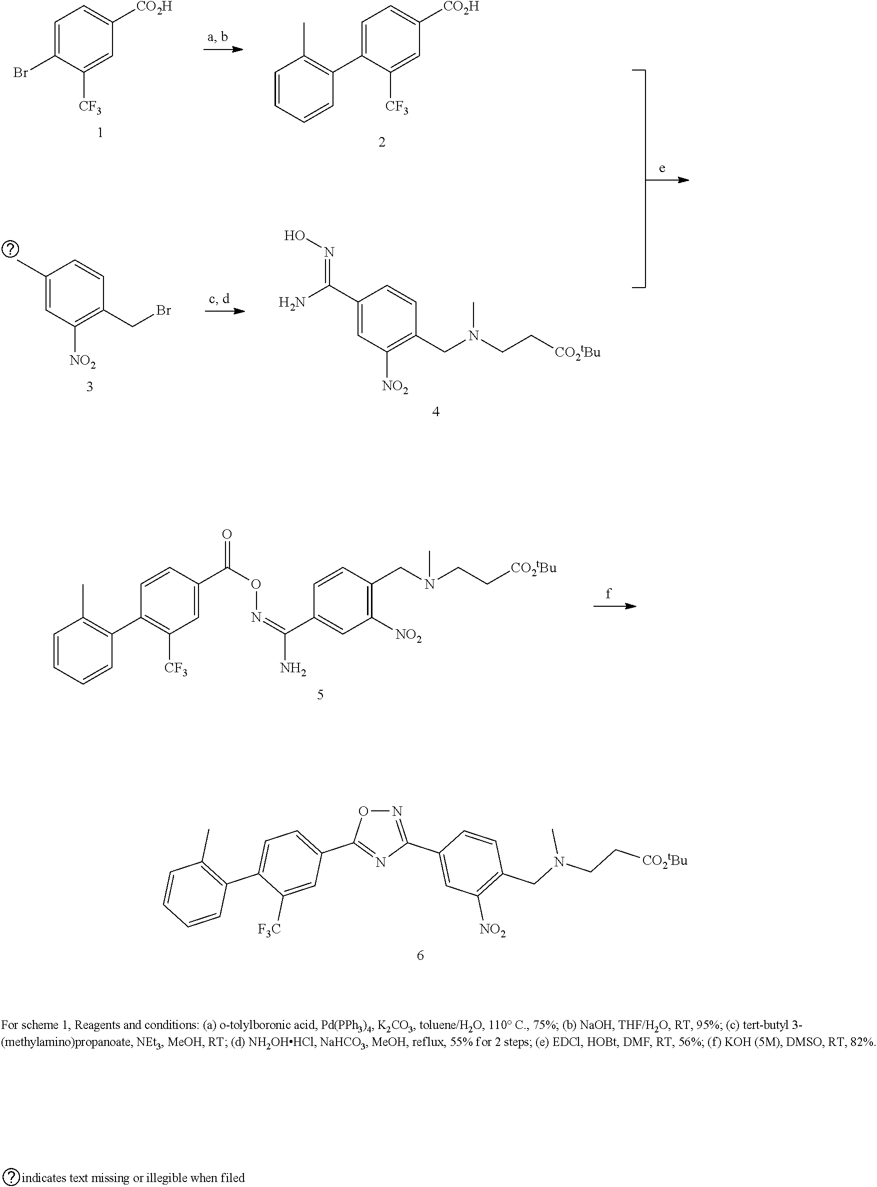 [SMILES: O=C(O)c1ccc(Br)c(C(F)(F)F)c1 ; Cc1ccccc1-c1ccc(C(=O)O)cc1C(F)(F)F ; Cc1ccc(CBr)c([N+](=O)[O-])c1 ; CN(CCC(=O)OC(C)(C)C)Cc1ccc(/C(N)=N/O)cc1[N+](=O)[O-] ; Cc1ccccc1-c1ccc(C(=O)O/N=C(/N)c2ccc(CN(C)CCC(=O)OC(C)(C)C)c([N+](=O)[O-])c2)cc1C(F)(F)F ; Cc1ccccc1-c1ccc(-c2nc(-c3ccc(CN(C)CCC(=O)OC(C)(C)C)c([N+](=O)[O-])c3)no2)cc1C(F)(F)F]

Compound 2 was prepared from 4-bromo-3-(trifluoromethyl)benzoic acid 1, which underwent Suzuki cross-coupling with ortho-tolylboronic acid and subsequent base hydrolysis (A. Quattropani, et al., ChemMedChem, 2015, 10, 688-714.). Nucleophilic substitution between 4-(bromomethyl)-3-nitrobenzonitrile 3 (C. H. Jin, et al., J. Med. Chem., 2014, 57, 4213-4238.) and tert-butyl 3-(methylamino)propanoate, followed by treatment with hydroxylamine hydrochloride in the presence of sodium bicarbonate yielded the intermediate amidoxime 4. Compound 2 was coupled with 4 to generate the uncycled intermediate 5 at room temperature. Then potassium hydroxide was used to promote the intramolecular cyclization of 5 to provide the nitro oxadiazole compound 6 in 82% yield (S. Baykov, et al., Tetrahedron Lett., 2016, 57, 2898-2900.).

However, starting with either precursor 5 or 6 to synthesize the key ¹⁸F-intermediate through the nitro/[¹⁸F]F⁻ replacement reaction was not successful although a variety of conditions were tested. The decomposing of 5 or 6 is faster than radiofluorination at the test reaction conditions. A multiple step ¹⁸F-labeling procedure was developed to produce [¹⁸F]FS1P1, via two sequential reductive amination reactions (Scheme 2). Scheme 2, below, shows an indirect approach plan for [¹⁸F]FS1P1 by sequential reductive amination reactions:

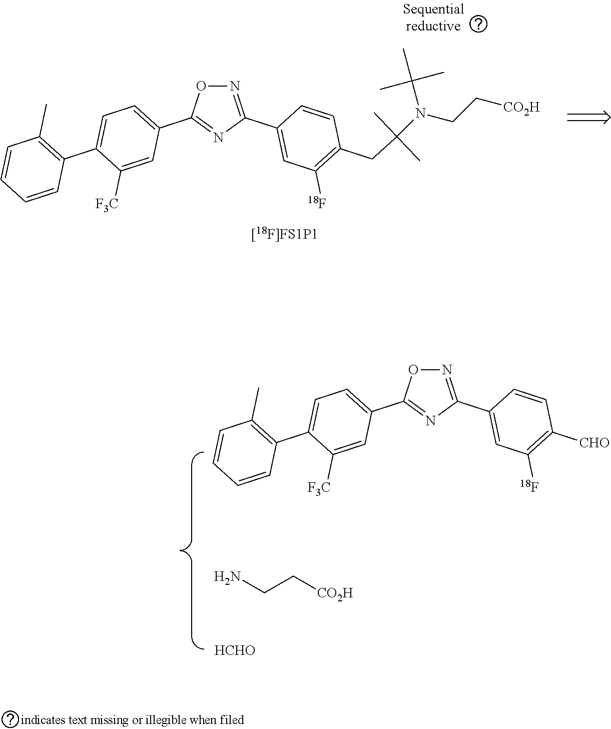 [SMILES: Cc1ccccc1-c1ccc(-c2nc(-c3ccc(CC(C)(C)N(CCC(=O)O)C(C)(C)C)c([18F])c3)no2)cc1C(F)(F)F ; Cc1ccccc1-c1ccc(-c2nc(-c3ccc(C=O)c([18F])c3)no2)cc1C(F)(F)F ; NCCC(=O)O ; "HCHO"]

The critical ortho-nitrobenzaldehyde precursors 10 and 11 were made as shown in Scheme 3, below (D. van der Born, et al., Chem. Soc. Rev., 2017, 46, 4709-4773; C. Lemaire, et al., J. Fluorine Chem., 2012, 138, 48-55; B. Shen, et al., J. Fluorine Chem., 2009, 130, 216-224; B. Shen, et al., J. Fluorine Chem., 2007, 128, 1461-1468.).

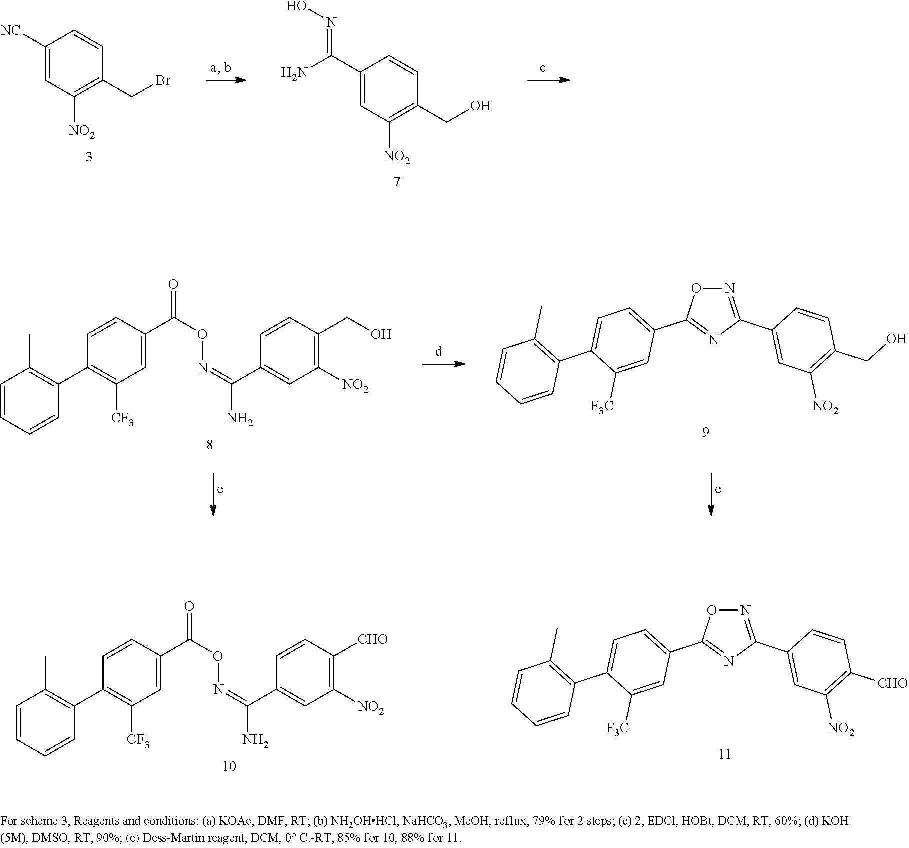 [SMILES: N#Cc1ccc(CBr)c([N+](=O)[O-])c1 ; N/C(=N\O)c1ccc(CO)c([N+](=O)[O-])c1 ; Cc1ccccc1-c1ccc(C(=O)O/N=C(/N)c2ccc(CO)c([N+](=O)[O-])c2)cc1C(F)(F)F ; Cc1ccccc1-c1ccc(-c2nc(-c3ccc(CO)c([N+](=O)[O-])c3)no2)cc1C(F)(F)F ; Cc1ccccc1-c1ccc(C(=O)O/N=C(/N)c2ccc(C=O)c([N+](=O)[O-])c2)cc1C(F)(F)F ; Cc1ccccc1-c1ccc(-c2nc(-c3ccc(C=O)c([N+](=O)[O-])c3)no2)cc1C(F)(F)F]

Firstly, amidoxime 7 was prepared from 4-(bromomethyl)-3-nitrobenzonitrile 3 by nucleophilic substitution with potassium acetate, followed by treating with hydroxylamine hydrochloride in the presence of sodium bicarbonate. Using a similar protocol of making compounds 5 and 6, the uncycled nitro benzyl alcohol 8, and its cycled compound 9 were produced at room temperature. The oxidation of 8 and 9 using Dess-Martin reagent afforded the substituted ortho-nitrobenzaldehyde precursors 10 and 11, and they were used as precursors for preparing [¹⁸F]FS1P1.

The scheme for optimization is as follows:

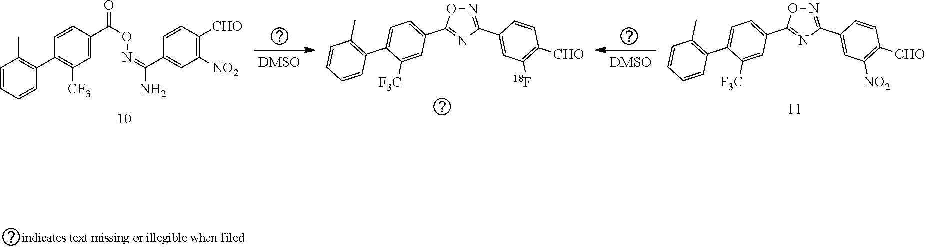 [SMILES: Cc1ccccc1-c1ccc(C(=O)O/N=C(/N)c2ccc(C=O)c([N+](=O)[O-])c2)cc1C(F)(F)F ; Cc1ccccc1-c1ccc(-c2nc(-c3ccc(C=O)c([18F])c3)no2)cc1C(F)(F)F ; Cc1ccccc1-c1ccc(-c2nc(-c3ccc(C=O)c([N+](=O)[O-])c3)no2)cc1C(F)(F)F]

TABLE 2
Optimization of Optimization of ¹⁸F-radiolabeling
conditions from ortho-nitro benzaldehyde 10 or 11.^{a}

| Entry | 10 or 11 (mg) | Additive (μL) | T (° C.) | [¹⁸F]12 RCY (%)^{b} |
|---|---|---|---|---|
| 1 | 11 (4.5) | none | 150 | 0 |
| 2 | 11 (9.2) | none | 150 | <5 |
| 3 | 10 (9.5) | none | 150 | <5 |
| 4 | 11 (18.2) | none | 150 | 50 ± 5 |
| 5 | 10 (18.5) | none | 150 | 55 ± 8 |
| 6 | 10 (4.5) | DABCO (10) | 150 | 45 ± 13 |
| 7 | 10 (4.5) | DBU (10) | 150 | 30 ± 6 |
| 8 | 10 (4.5) | TMEDA (10) | 150 | 65 ± 10 |
| 9 | 10 (4.5) | DIPEA (10) | 150 | 43 ± 12 |
| 10 | 10 (4.5) | TMEDA (10) | 120 | 20 ± 5 |
| 11 | 10 (2.3) | TMEDA (10) | 150 | 60 ± 13 |
| 12 | 10 (1.2) | TMEDA (10) | 150 | 12 ± 3 |
| 13^{c} | 10 (4.5) | TMEDA (30) | 150 | 70 ± 16 |

^{a}[¹⁸F]KF/K₂₂₂, K₂CO₃, DMSO (300 μL), 5 min.
^{b}RCY was determined based on radio = TLC (n = 3).
^{c}1 μL H₂O added. DABCO: 1,4-diaza[2.2.2]bicyclooctane; DBU: 1,8-diazabicyclo[5.4.0]undec-7-ene; TMEDA: N,N,N′,N′-tetramethylethylenediamine; DIPEA: N,N-diisopropylethylamine.

To radiosynthesize [¹⁸F]FS1P1, the initial focus was on optimizing conditions to improve the yield of making substituted ortho-[¹⁸F]fluorobenzaldehyde [¹⁸F]12 through the NO₂/[¹⁸F]/F⁻ replacement of the precursors 10 or 11. As shown in Table 2, when directly using substituted ortho-nitrobenzaldehyde 11 (4 mg) reacting with [¹⁸F]KF in DMSO at 150° C., no product of [¹⁸F]12 was detected by radioactive TLC (entry 1). Further analysis indicated that both the uncycled precursor 10 and cycled precursor 11 were easily decomposed under high temperature that reduced the radiochemical yield for [¹⁸F]12. Therefore, the amount of precursor was increased to increase radiochemical yield (entries 2-5). Using 18 mg of the substituted ortho-nitrobenzaldehyde 10 or 11 led to a radiochemical yield of [¹⁸F]12 that was ˜50% based on radioactive TLC monitoring (entry 4-5). Nevertheless, the purification of the radioactive product [¹⁸F]12 through high-performance liquid chromatography (HPLC) became challenging because the excess amount of precursor liquid chromatography (HPLC) became challenging because the excess amount of precursor 10 or 11 generated a substantial mass of side products. The tertiary amines such as 1,4-diaza[2.2.2]bicyclooctane (DABCO) have been reported to enhance the nucleophilic radiofluorination efficiency (A. B. Gómez, et al., Chem. Comm., 2016, 52, 13963-13966; G. R. Naumiec, et al., Eur. J. Org. Chem., 2017, 2017, 6593-6603; S. J. Lee, et al., J. Org. Chem., 2021, 86, 14121-14130.).

Therefore, DABCO was added to the radiolabeling reaction vial (entry 6). Starting with 4 mg of the uncycled precursor 10 at the presence of DABCO, gave 45% radiochemistry yield of [¹⁸F]12 (entry 6 versus entry 3). Inspired by the remarkable efficiency of DABCO addition, different tertiary amine additives including 1,8-diazabicyclo[5.4.0]undec-7-ene (DBU), N,N,N′,N′-tetramethyl-ethylenediamine (TMEDA), N,N-diisopropylethylamine (DIPEA) were further tested (entries 7-9). The experiment data suggested that TMEDA was the optimal additive to prepare [¹⁸F]12 with 65±10% radiochemical yield (entry 8). Further condition testing by lowering reaction temperature or decreasing precursor loading did not improve the reaction output (entry 10-12). In the end, the optimized reaction condition for [¹⁸F]12 was determined (entry 13), starting with ˜4.0 mg of precursor 10, utilizing TMEDA (30 μL) as an additive, combined with H₂O (1 μL) and heating 5 min at 150° C. in DMSO (300 μL), the synthesis of [¹⁸F]12 was accomplished in 70% radioactive TLC yield. Furthermore, organic solvent ether extraction was employed to replace the HPLC separation for purification of the intermediate [¹⁸F]12, which reduced the total time for the whole radiosynthesis procedure. Together, utilizing a small amount of precursor 10 combined with TMEDA as the additive led to a significant improvement of this radiolabeling procedure and resolved the challenge of [¹⁸F]12 purification by HPLC.

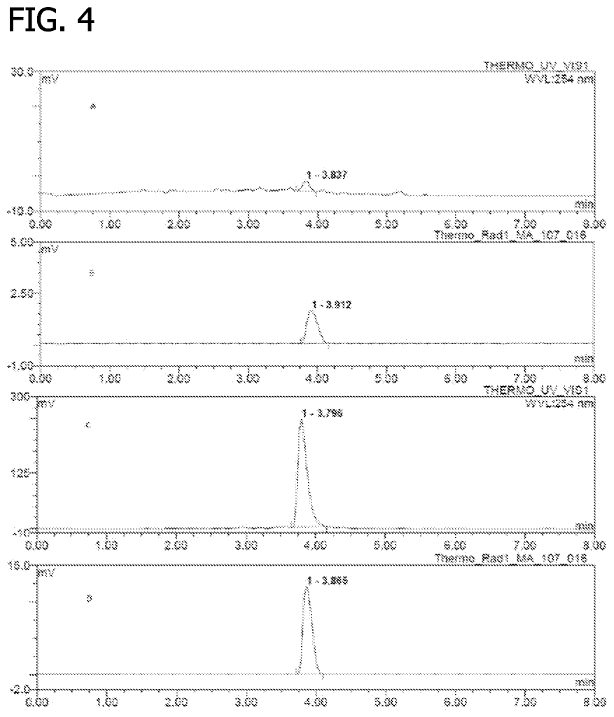

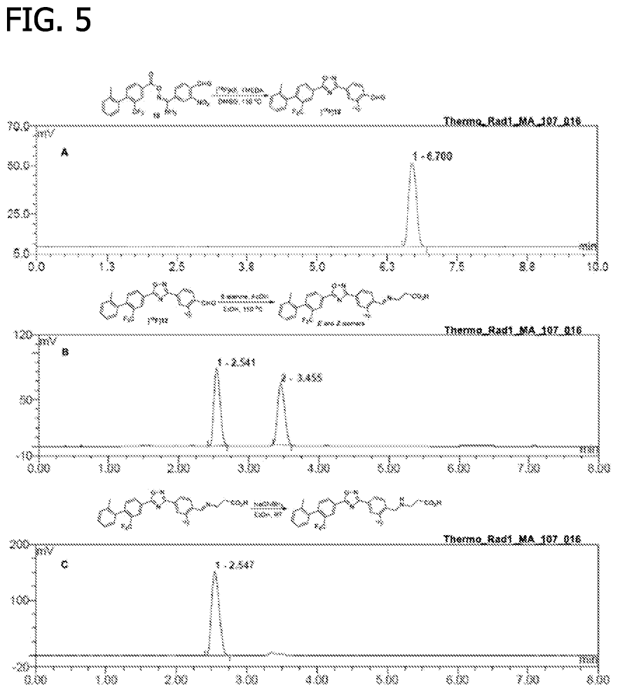

After a reliable procedure of synthesizing and purifying the key intermediate [¹⁸F]12 was established, a multiple step F-18 labeling strategy was further explored for making [¹⁸F]FS1P1 as shown in Scheme 4, below.

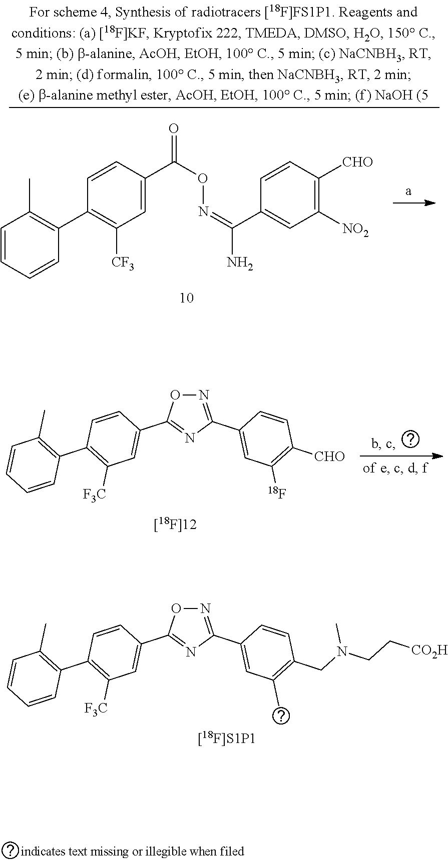 [SMILES: Cc1ccccc1-c1ccc(C(=O)O/N=C(/N)c2ccc(C=O)c([N+](=O)[O-])c2)cc1C(F)(F)F ; Cc1ccccc1-c1ccc(-c2nc(-c3ccc(C=O)c([18F])c3)no2)cc1C(F)(F)F ; Cc1cc(-c2noc(-c3ccc(-c4ccccc4C)c(C(F)(F)F)c3)n2)ccc1CN(C)CCC(=O)O]

[¹⁸F]12 firstly went through reductive amination by reacting with β-alanine, and then methylation by treating with formalin in one pot (See FIG. 4-FIG. 5), the radiotracer [¹⁸F]FS1P1 was obtained with 10% radiochemical yield from [¹⁸F]12 (decay corrected to end of synthesis). However, substituted ortho-[¹⁸F]fluorobenzyl alcohol was the major radioactive product when purifying [¹⁸F]FS1P1. The lower solubility of β-alanine in ethanol may lower the conversion rate from [¹⁸F]12 to the imine; instead the aldehyde intermediate [¹⁸F]12 was mainly reduced to the corresponding ortho-[¹⁸F]fluorobenzyl alcohol as a side product. To address this issue, the β-alanine methyl ester was used to replace β-alanine, and no side products of ortho-[¹⁸F]fluorobenzyl alcohol were found. Subsequently, using aqueous sodium hydroxide solution to hydrolyze the methyl ester yielded the intended radioactive product [¹⁸F]FS1P1. After purification using a reverse-phase HPLC system, the final product [¹⁸F]FS1P1 was obtained with 30-50% radiochemical yield, >95% chemical and radiochemical purity, and a high molar activity (37-166.5 GBq/μmol, decay corrected to end of synthesis, EOS). It took approximately 120 min from drying the [¹⁸F]KF to formulate the radioactive dose of [¹⁸F]FS1P1 for in vitro and in vivo animal study. The lipophilicity of [¹⁸F]FS1P1 was measured using 1-octanol/PBS buffer method and the Log D_{7.4} value was 2.62±0.31 (n=3), suggesting a good blood-brain barrier (BBB) permeability.

Further details on the synthesis of compound 12 are described below, in Scheme S1.

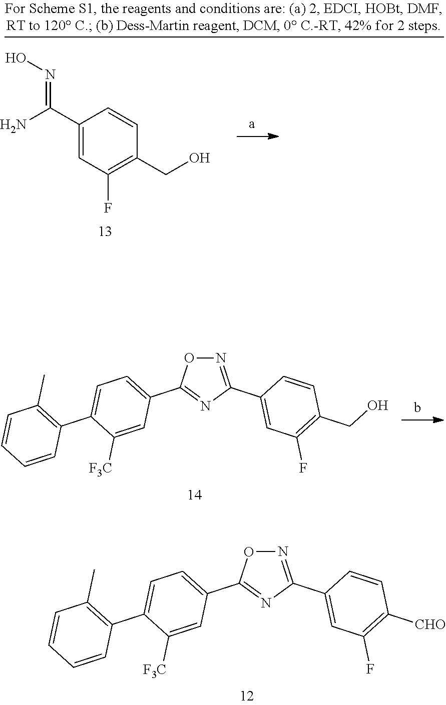 [SMILES: N/C(=N\O)c1ccc(CO)c(F)c1 ; Cc1ccccc1-c1ccc(-c2nc(-c3ccc(CO)c(F)c3)no2)cc1C(F)(F)F ; Cc1ccccc1-c1ccc(-c2nc(-c3ccc(C=O)c(F)c3)no2)cc1C(F)(F)F]

To a round-bottom flask equipped with a stir bar was added acid 2 (280 mg, 1.0 mmol), HOBt (135 mg, 1.0 mmol), EDCI (287 mg, 1.5 mmol), and DMF (10 mL). The reaction was stirred for 0.5 h followed by adding amidoxime 13 (220 mg 1.2 mmol). Then, the reaction mixture was stirred at 120° C. for 2 h and monitored by TLC. After reaction finished, the reaction mixture was diluted with water and extracted with ethyl acetate. The ethyl acetate layer was washed with saturated brine, and dried over anhydrous MgSO₄. After filtration and concentration, the crude residue was used directly for next step without further purification.

The crude residue was dissolved in dichloromethane (10 mL). Dess-Martin reagent (508 mg, 1.0 mmol) was added to the reaction solution at 0° C. Then, the reaction mixture was stirred at room temperature and monitored by TLC. After accomplishment, the reaction was diluted with dichloromethane and water, the separated dichloromethane layer was washed with saturated brine and dried over anhydrous MgSO4. After filtering and concentrated in vacuum, the crude residue was purified on a silica gel column to afford 12 (180 mg, yield 42%).

Further details on these methods and NMR data on these compounds are described below.

All reagents and chemicals were purchased commercially and used as received, unless otherwise stated. Reactions were monitored by thin-layer chromatography (TLC) using silica gel 60 F254 (EMD Chemicals Inc, Billerica, MA). Flash column chromatography was conducted using 230-400 mesh silica gel (SiliCycle Inc, Quebec, Canada). Melting points were determined on a MEL-TEMP 3.0 apparatus without correction. All deuterated solvents were purchased from Cambridge Isotope Laboratories. ¹H and ¹³C NMR spectra were recorded on a 400 MHz Varian instrument. Chemical shifts were reported in parts per million (ppm) and were calibrated using a residual undeuterated solvent as an internal reference (CDCl₃: δ 7.26 ppm; CD₃OD: δ 3.31 ppm; Acetone-d6: δ 2.05 ppm; DMSO-d6: δ 2.50 ppm). Coupling constants (J) are reported in Hertz (Hz). Multiplicities are indicated by s (singlet), d (doublet), t (triplet), q (quartet), p (pentet), h (hextet), m (multiplet) and br (broad). High-resolution positive ion mass was acquired by a Bruker MaXis 4G Q-TOF mass spectrometer with an electrospray ionization source. The Accell Plus QMA Cartridge (Inc Catalog #WAT023525), sodium sulfate Sep-Pak cartridges (Inc Catalog #WAT054265), and C18 Sep-Pak Plus cartridges (Inc Catalog #WAT020515) were purchased from Waters Corporation, Milford, MA.

### 2′-Methyl-2-(trifluoromethyl)-[1,1′-biphenyl]-4-carboxylic acid (2)

Synthesis of compound 2 followed the published procedure (A. Quattropani, et al., ChemMedChem, 2015, 10, 688-714.).

### Tert-butyl (Z)-3-((4-(N′-hydroxycarbamimidoyl)-2-nitrobenzyl)-(methyl)amino)propanoate (4)

To a round-bottom flask equipped with a stir bar was added 4-(bromomethyl)-3-nitrobenzonitrile 3 (1.20 g, 5.0 mmol), tert-butyl 3-(methylamino)propanoate (0.95 g, 6.0 mmol), and methanol (20 mL). After cooling to 0° C., triethylamine (1.01 g, 10.0 mmol) was added to the mixture dropwise. The mixture was warmed to RT and stirred overnight. Then, the mixture was concentrated in vacuum, and the crude residue was used directly without further purification.

To a round-bottom flask equipped with a stir bar and the crude residue was added hydroxylamine hydrochloride (0.70 g, 10 mmol), NaHCO₃ (1.68 g, 20 mmol), and methanol (20 mL). The reaction was refluxed and stirred in a pre-heated 70° C. oil-bath for 8 h. The reaction mixture was cooled to room temperature, and the precipitate was filtered off and washed with methanol. The filtrate was concentrated under reduced pressure, and the crude residue was purified by flash chromatography, eluted with ethyl acetate/methanol (3/2, v/v) to afford the product 4 (1.05 g). Yield: 55%, yellow oil. ¹H NMR (400 MHz, DMSO-d6) δ 9.73 (s, 1H), 7.46 (d, J=8.0 Hz, 1H), 7.39 (d, J=11.5 Hz, 1H), 7.33 (t, J=7.6 Hz, 1H), 5.83 (s, 2H), 3.47 (s, 2H), 2.55 (t, J=6.5 Hz, 2H), 2.33 (t, J=6.6 Hz, 2H), 2.08 (s, 3H), 1.34 (s, 9H); ¹³C NMR (101 MHz, DMSO-d6) δ 171.62, 162.03, 150.04, 134.61, 131.37, 126.10, 121.28, 112.18, 79.89, 53.93, 52.77, 41.89, 33.92, 28.12.

### Tert-butyl (Z)-3-(methyl(4-(N′-((2′-methyl-2-(trifluoromethyl)-[1,1′-biphenyl]-4-carbonyl)oxy)carb-amimidoyl)-2-nitro-benzyl)-amino)propanoate (5)

To a round-bottom flask equipped with a stir bar was added acid 2 (280 mg, 1.0 mmol), HOBt (135 mg, 1.0 mmol), EDCI (287 mg, 1.5 mmol), and DMF (10 mL). The reaction mixture was stirred for 0.5 h followed by adding amidoxime 4 (422 mg, 1.2 mmol). The reaction mixture was stirred overnight at room temperature and monitored by TLC. After finish, the reaction mixture was diluted with water and extracted with ethyl acetate. The ethyl acetate layer was washed with saturated brine, and dried over anhydrous MgSO4. After filtration and concentration, the residue was purified on a silica gel column to afford 5. Yield: 56%, yellow oil. ¹H NMR (400 MHZ, CDCl₃) δ 8.37 (s, 1H), 8.19 (d, J=7.8 Hz, 1H), 8.11 (s, 1H), 7.86 (d, J=8.0 Hz, 1H), 7.64 (d, J=8.1 Hz, 1H), 7.30 (d, J=7.9 Hz, 1H), 7.22 (d, J=7.3 Hz, 1H), 7.20-7.09 (m, 2H), 7.02 (d, J=7.4 Hz, 1H), 5.69 (s, 2H), 3.72 (s, 2H), 2.61 (t, J=6.8 Hz, 2H), 2.31 (q, J=7.1 Hz, 2H), 2.08 (s, 3H), 1.94 (d, J=4.0 Hz, 3H), 1.35 (s, 9H); ¹³C NMR (101 MHz, CDCl₃) δ 203.84, 171.83, 162.83, 155.57, 149.26, 145.75 (d, J_{C-F}=2.0 Hz), 137.68 (d, J_{C-F}=3.0 Hz), 135.45, 132.29 (d, J_{C-F}=4.0 Hz), 132.16, 131.31, 130.85, 130.75, 129.72, 129.33 (d, J_{C-F}=30.3 Hz), 128.85, 128.60, 128.36, 127.24 (d, J_{C-F}=6.0 Hz), 124.96, 124.72, 123.36 (q, J_{C-F}=275.7 Hz), 122.84 (d, J_{C-F}=2.0 Hz), 80.51, 58.17, 53.15, 42.07, 42.04, 33.86, 28.02, 19.97; HRMS (ESI) calculated for C₃₁H₃₄F₃N4O6 [M+H]+ 615.2430, found 615.2425.

### Tert-butyl 3-(methyl(4-(5-(2′-methyl-2-(trifluoro methyl)-[1,1′-biphenyl]-4-yl)-1,2,4- oxadiazol-3-yl)-2-nitro-benzyl)amino)-propanoate (6)

To a round-bottom flask equipped with a stir bar was added 5 (307 mg, 0.5 mmol) and DMSO (5 mL). A solution of potassium hydroxide (0.6 mL, 5 M) was added dropwise. The reaction mixture was stirred for 0.5 h at room temperature and monitored by TLC. The reaction was diluted with ethyl acetate and water, the ethyl acetate layer was washed with saturated brine and dried over anhydrous MgSO₄. After filtering and concentrated in vacuum, the crude residue was purified on a silica gel column to afford 6. Yield: 82%, yellow oil. ¹H NMR (400 MHZ, Acetone-d6) δ 8.45 (s, 1H), 8.41 (s, 1H), 8.36 (d, J=7.8 Hz, 1H), 8.26-8.19 (m, 1H), 7.81 (d, J=8.0 Hz, 1H), 7.49 (d, J=7.9 Hz, 1H), 7.21 (q, J=7.4 Hz, 2H), 7.13 (t, J=7.1 Hz, 1H), 7.04 (d, J=7.4 Hz, 1H), 3.75 (s, 2H), 2.58 (t, J=7.1 Hz, 2H), 2.26 (t, J=7.1 Hz, 2H), 2.08 (s, 3H), 1.93 (s, 3H), 1.29 (s, 9H); ¹³C NMR (101 MHz, Acetone-d6) δ 174.93, 171.04, 167.33, 150.10, 145.50, 137.92, 137.60, 135.40, 133.42 (d, J_{C-F}=5.0 Hz), 132.17, 131.16, 130.66, 129.78, 129.40 (d, J_{C-F}=31.3 Hz), 128.84, 128.53, 126.63, 125.64 (q, J_{C-F}=5.0 Hz), 125.09, 123.50 (q, J_{C-F}=275.7 Hz), 123.44, 122.88, 79.48, 58.01, 53.07, 41.59, 41.54, 33.46, 27.40, 19.30, 19.27; HRMS (ESI) calculated for C₃₁H₃₂F₃N₄O₅ [M+H]+ 597.2325, found 597.2321.

### (Z)-N′-hydroxy-4-(hydroxymethyl)-3-nitrobenzimid amide (7)

To a round-bottom flask equipped with a stir bar was added 4-(bromomethyl)-3-nitrobenzonitrile 3 (1.20 g, 5.0 mmol), potassium acetate (0.74 g, 7.5 mmol), and DMF (20 mL). The mixture was stirred overnight at RT. Then, the reaction mixture was diluted with water and extracted with ethyl acetate. The ethyl acetate layer was washed with saturated brine, and dried over anhydrous MgSO₄. After filtration and concentration, the residue was used directly without further purification.

To a round-bottom flask equipped with a stir bar and the crude residue was added hydroxylamine hydrochloride (0.70 g, 10 mmol), NaHCO₃ (1.68 g, 20 mmol), and methanol (20 mL). The reaction was refluxed and stirred in a pre-heated 70° C. oil-bath for 8 h. The reaction mixture was cooled to room temperature, and the precipitate was filtered off and washed with methanol. The filtrate was concentrated under reduced pressure, and the crude residue was purified by flash chromatography, eluted with ethyl acetate/methanol (3/1, v/v) to afford the product 7 (0.83 g), Yield: 79%, yellow solid, melting point (MP): 101-103° C. ¹H NMR (400 MHZ, DMSO-d6) δ 9.87 (s, 1H), 8.29 (s, 1H), 8.01 (d, J=8.1 Hz, 1H), 7.79 (d, J=8.1 Hz, 1H), 6.02 (s, 2H), 5.56 (t, J=5.2 Hz, 1H), 4.79 (d, J=4.8 Hz, 2H); ¹³C NMR (101 MHz, DMSO-d6) δ 149.41, 147.05, 139.10, 133.37, 130.49, 128.69, 121.38, 60.26.

### (Z)-4-(hydroxymethyl)-N′-((2′-methyl-2-(trifluoromethyl)-[1,1′-biphenyl]-4-carbonyl)oxy)-3-nitro benzimidamide (8)

To a round-bottom flask equipped with a stir bar was added acid 2 (280 mg, 1.0 mmol), HOBt (135 mg, 1.0 mmol), EDCI (287 mg, 1.5 mmol), and dichloromethane (10 mL). The reaction mixture was stirred for 0.5 h followed by adding amidoxime 7 (253 mg 1.2 mmol). The reaction mixture was stirred for overnight at room temperature and monitored by TLC. After finish, the reaction mixture was diluted with water and extracted with ethyl acetate. The ethyl acetate layer was washed with 1 M HCl, saturated brine, and dried over anhydrous MgSO₄. After filtration and concentration, the residue was purified on a silica gel column to afford 8. Yield: 60%, yellow solid, MP: 151-154° C. ¹H NMR (400 MHZ, CD₃OD) δ 8.54 (d, J=7.9 Hz, 2H), 8.47 (d, J=6.7 Hz, 1H), 8.16 (d, J=7.5 Hz, 1H), 7.99 (d, J=7.4 Hz, 1H), 7.46 (d, J=7.4 Hz, 1H), 7.30 (s, 2H), 7.23 (s, 1H), 7.12 (s, 1H), 5.01 (s, 2H), 2.04 (s, 3H); ¹³C NMR (101 MHz, CD₃OD) δ 163.60, 156.89, 147.02, 145.62, 140.50, 137.88, 135.25, 132.32, 131.63, 131.27, 129.39, 128.89 (d, J_{C-F}=31.3 Hz), 128.69, 128.58, 128.38, 128.07, 126.90 (q, J_{C-F}=5.0 Hz), 124.67, 123.56 (d, J_{C-F}=274.7 Hz), 123.06, 60.37, 18.74.

### (4-(5-(2′-Methyl-2-(trifluoromethyl)-[1,1′-biphenyl]-4-yl)-1,2,4-oxadiazol-3-yl)-2-nitro-phenyl)methanol (9)

To a round-bottom flask equipped with a stir bar was added 8 (473 mg, 1.0 mmol) and DMSO (10 mL). A solution of potassium hydroxide (1.2 mL, 5 M) was added dropwisely. The reaction mixture was stirred for 0.5 h at room temperature and monitored by TLC. The reaction was diluted with ethyl acetate and water, the ethyl acetate layer was washed with saturated brine and dried over anhydrous MgSO₄. After filtering and concentrated in vacuum, the crude residue was purified on a silica gel column to afford 9. Yield: 90%, yellow solid, MP: 143-146° C. ¹H NMR (400 MHZ, Acetone-d6) δ 8.70 (d, J =1.3 Hz, 1H), 8.56 (s, 1H), 8.51-8.35 (m, 2H), 8.15 (d, J=8.1 Hz, 1H), 7.61 (d, J=7.9 Hz, 1H), 7.39-7.29 (m, 2H), 7.26 (t, J=7.2 Hz, 1H), 7.18 (d, J=7.5 Hz, 1H), 5.06 (s, 2H), 4.85 (s, 1H), 2.06 (d, J=4.8 Hz, 3H); ¹³C NMR (101 MHz, Acetone-d6) δ 174.88, 167.31, 147.27, 145.48, 141.98, 137.59, 135.42, 133.40 (d, J_{C-F}=5.0 Hz), 131.67, 131.11, 129.77, 129.38 (d, J_{C-F}=30.3 Hz), 129.34, 128.85, 128.53, 126.16, 125.59 (q, J_{C-F}=5.0 Hz), 125.08, 123.50 (q, J_{C-F}=275.7 Hz), 123.38, 122.96 (d, J_{C-F}=3.0 Hz), 60.68, 19.27 (d, J_{C-F}=4.0 Hz).

### (Z)-4-formyl-N′-((2′-methyl-2-(trifluoromethyl)-[1,1′-biphenyl]-4-carbonyl)oxy)-3-nitrobenzimidamide (10)

To a round-bottom flask equipped with a stir bar was added 8 (237 mg, 0.5 mmol) and dichloromethane (5 mL). The reaction mixture was stirred at 0° C. for 0.5 h followed by adding Dess-Martin reagent (254 mg, 0.6 mmol). The reaction mixture was stirred at room temperature and monitored by TLC. After finish, the reaction was diluted with dichloromethane and water, the ethyl acetate layer was washed with saturated brine and dried over anhydrous MgSO₄. After filtering and concentrated in vacuum, the crude residue was purified on a silica gel column to afford 10. Yield: 85%, yellow solid, MP: 160-163° C. ¹H NMR (400 MHZ, Acetone-d6) δ 10.43 (s, 1H), 8.63 (s, 1H), 8.55 (s, 1H), 8.50 (d, J=7.9 Hz, 1H), 8.42 (d, J=8.0 Hz, 1H), 8.08 (d, J=8.0 Hz, 1H), 7.52 (d, J=7.9 Hz, 1H), 7.35 (d, J=6.9 Hz, 2H), 7.27 (t, J=7.0 Hz, 1H), 7.17 (d, J=7.5 Hz, 1H), 7.06 (s, 2H), 2.06 (s, 3H); ¹³C NMR (101 MHz, Acetone-d6) δ 189.12, 163.05, 155.93, 150.46, 146.21, 142.09, 138.85, 137.89, 136.27, 133.65, 133.48, 133.37, 132.89, 130.82, 130.65, 130.60, 130.16, 129.75, 129.46 (d, J_{C-F}=30.3 Hz), 129.26, 128.04 (d, J_{C-F}=6.0 Hz), 125.92, 124.65 (d, J_{C-F}=274.7 Hz), 123.66, 20.15; HRMS (ESI) calculated for C₂₃H₁₇F₃N₃O₅ [M+H]+ 472.1120, found 472.1115.

### 4-(5-(2′-Methyl-2-(trifluoromethyl)-[1,1′-biphenyl]-4-yl)-1,2,4-oxadiazol-3-yl)-2-nitrobenz aldehyde (11)

To a round-bottom flask equipped with a stir bar was added 9 (228 mg, 0.5 mmol) and dichloromethane (5 mL). The reaction mixture was stirred at 0° C. for 0.5 h followed by adding Dess-Martin reagent (254 mg, 0.6 mmol). The reaction mixture was stirred at room temperature and monitored by TLC. After finish, the reaction was diluted with dichloromethane and water, the ethyl acetate layer was washed with saturated brine and dried over anhydrous MgSO₄. After filtering and concentrated in vacuum, the crude residue was purified on a silica gel column to afford 11. Yield: 88%, yellow solid, MP: 152-155° C. ¹H NMR (400 MHZ, Acetone-d6) δ 10.46 (s, 1H), 8.85 (d, J=1.0 Hz, 1H), 8.74-8.62 (m, 2H), 8.57 (d, J=7.9 Hz, 1H), 8.19 (d, J=7.8 Hz, 1H), 7.68 (d, J=7.9 Hz, 1H), 7.37 (d, J=7.6 Hz, 2H), 7.30 (t, J=7.1 Hz, 1H), 7.21 (d, J=7.4 Hz, 1H), 2.09 (s, 3H); ¹³C NMR (101 MHz, Acetone-d6) δ 189.04, 176.32, 167.87, 151.01, 146.61, 138.49, 136.31, 134.43, 134.08, 133.10, 132.46, 132.18, 131.64, 130.71, 130.36 (d, J_{C-F}=30.3 Hz), 129.75, 129.47, 126.65 (q, J_{C-F}=5.0 Hz), 126.02, 124.41 (q, J_{C-F}=274.7 Hz), 124.23, 123.86, 20.14; HRMS (ESI) calculated for C₂₃H₁₅F₃N₃O₄ [M+H]+ 454.1015, found 454.1009.

### 2-Fluoro-4-(5-(2′-methyl-2-(trifluoromethyl)-[1,1′-biphenyl]-4-yl)-1,2,4-oxadiazol-3-yl)benzaldehyde (12)

Synthesis of compound 12 followed the procedure in Electronic Supplementary Material (Scheme S1).

Yield: 53%, white solid, MP: 121-123° C. ¹H NMR (400 MHZ, Acetone-d6) δ 10.38 (s, 1H), 8.61 (s, 1H), 8.52 (d, J=8.0 Hz, 1H), 8.16 (d, J=8.0 Hz, 1H), 8.11-7.97 (m, 2H), 7.66 (d, J=7.9 Hz, 1H), 7.41-7.31 (m, 2H), 7.27 (t, J=7.1 Hz, 1H), 7.19 (d, J=7.5 Hz, 1H), 2.06 (s, 3H); ¹³C NMR (101 MHZ, Acetone-d6) δ 186.22 (d, J_{C-F}=6.0 Hz), 175.13, 167.43, 164.13 (d, J_{C-F}=258.6 Hz), 145.54, 137.58, 135.41, 133.74 (d, J_{C-F}=10.1 Hz), 133.48, 131.20, 129.88 (d, J_{C-F}=2.0 Hz), 129.79, 129.38 (d, J_{C-F}=30.3 Hz), 128.83, 128.56, 126.15 (d, J_{C-F}=9.0 Hz), 125.66 (q, J_{C-F}=6.0 Hz), 125.11, 124.87, 123.58 (d, J_{C-F}=4.0 Hz), 123.50 (q, J_{C-F}=275.7 Hz), 115.34 (d, J_{C-F}=24.2 Hz), 19.23; HRMS (ESI) calculated for C₂₃H₁₅F₄N₂O₂ [M+H]+ 427.1070, found 427.1067.

### Radiochemistry

Procedure for the Radiosynthesis of [¹⁸F]FS1P1 Mediated by [¹⁸F]12 (Scheme 4)

The [¹⁸F]fluoride in a 0.2˜2.5 mL bolus of [¹⁸O]H₂O and was trapped on a

pre-conditioned QMA cartridge (WAT023525, Waters) to remove [¹⁸O]H₂O and other aqueous impurities. [¹⁸F]Fluoride was eluted into the reaction vessel using aqueous potassium carbonate solution (3.0 mg/mL).

[¹⁸F]KF (˜7.4 GBq) aqueous solution was added to a vial containing Kryptofix 222 (K₂₂₂) (6˜7 mg), and dried by azeotropic evaporation with acetonitrile (3×1 mL) under N₂ flow at 100° C. The vial was cooled to room temperature, and a solution of the precursor 10 (4-5 mg) in DMSO (300 μL) and TMEDA (30 mg), H₂O (1 μL) were added and heated at 150° C. for 5 min, and then cooled to room temperature. The reaction mixture was diluted with 3 mL saturated sodium chloride solution and extracted with ether (3×2 mL). The ether solution was collected and then passed through two stacked plus long sodium sulfate Sep-Pak cartridges (WAT054265, Waters) to remove the residual water. After removing ether, using N₂ flow at room temperature, a solution of β-alanine methyl ester (5.0 mg) and acetic acid (5 μL) in anhydrous ethanol (300 μL) was added into the reaction vial and heated at 100° C. for 5 min. Upon cooling to room temperature, sodium cyanoborohydride (3 mg) was added to the reaction mixture. The vial was capped, shaken, and allowed to stand at room temperature for 2 min. Formalin (50 μL) was added to the reaction mixture. The vial was capped, shaken occasionally, and stand at room temperature for 2 min. Sodium cyanoborohydride (3 mg) was added to the reaction mixture. The vial was capped, shaken, and heated at 100° C. for 5 min. Upon cooling to room temperature, sodium hydroxide (100 μL, 5 M) was added to the reaction mixture. The vial was capped, shaken, and then heated at 100° C. for 5 min. After cooling by water bath, the reaction mixture was quenched by acetic acid (50 μL) and HPLC mobile phase (3.0 mL, 51% acetonitrile in 0.1 M ammonium formate buffer, pH=4.5). The solution was loaded onto a reverse semi-preparative HPLC system for purification. The HPLC system contains a 5 mL injection loop, a Phenomenex Luna column (250×9.6 mm, 5 μm), a UV detector at 254 nm wavelength, and a radioactivity detector. Using acetonitrile/0.1 M ammonium formate buffer (51/49, v/v, pH 4.5) as the eluent with a flow rate of 4 mL/min, the retention time of the radioactive product was collected from 25 to 28 min. The radioactive product fraction collection was diluted using sterile water (˜50 mL) and then passed through a C18 Sep-Pak Plus short cartridge (WAT020515, Waters). The trapped product was eluted using 10% ethanol in 0.9% saline. After sterile filtration into a glass vial, [¹⁸F]FS1P1 was ready for quality control (QC) analysis and animal studies. QC HPLC was conducted following the conditions: Phenomenex SB-C18 column (250×4.6 mm, 5 μm), mobile phase 75% acetonitrile in ammonium formate buffer (0.1 M, pH 4.5) as mobile phase, flow rate at 1.5 mL/min, UV wavelength at 254 nm, and tR at 3.8 min. The decay corrected radiochemical yield of making [¹⁸F]FS1P1 from [¹⁸F]/fluoride was 30-50% (decay corrected to the end of synthesis), with >95% chemical and radiochemical purity, and molar activity ranged from 37-166.5 GBq/μmol (1000-4500 Ci/mmol, decay corrected to the end of synthesis). The synthesis of [¹⁸F]FS1P1 took about 120 min including the [¹⁸F]fluorine drying step.

The other procedures for condition optimization of radiolabelling were similar with the process above.

### Log D_{7.4} Measurement

Partition coefficient was measured by mixing the [¹⁸F]FS1P1 sample with 3 mL each of 1-octanol and buffer that is 0.1 M phosphate and pH equals 7.4 in a test tube. The mixture in the test tube was vortexed for 20 sec followed by centrifugation for 1 min at room temperature. Then 2 mL of the organic layer was transferred to a second test tube, and 1 mL of 1-octanol and 3 mL of PBS buffer were added. The resulting mixture was vortexed for 20 sec, followed by centrifugation for 1 min at room temperature. Then 1 mL of the organic and aqueous layer were taken separately for measurement. The radioactivity content values (count per minute) of two samples (1 mL each) from the 1-octanol and buffer layers were counted using a gamma counter. The partition coefficient Log D_{7.4} was determined by calculating as the decimal logarithm the ratio of cpm/mL between 1-octanol and PBS buffer. The measurements were repeated three times. The value of the partition coefficient is 2.62±0.31.

S1P1 plays a crucial role in various physiological and pathophysiological processes. While most previous efforts aimed at the development of S1P1 specific ligands for improving their therapeutic effect, these efforts focused on the identification of a S1P1 specific radioligand for quantitative measurement of S1P1 expression in response to inflammation. A few of S1P1 specific radioligands and preclinical studies for rodent disease models including MS, carotid injury, vascular injury, and infection disease were previously reported. Importantly, with the FDA approval of [¹¹C]CS1P1 for human use, whole body dosimetry studies and tissue distribution studies in 10 human subjects were completed, suggesting [¹¹C]CS1P1 is safe for investigating S1P1 expression for human CNS disorders and other diseases. Nevertheless, an F-18 labeled S1P1 specific radiotracer may offer many advantages for clinical use and facilitate multiple center clinical trial studies of neuroinflammation in CNS and peripheral tissues. Because the structure of the CS1P1 molecule contains a fluorine atom in one of the three aromatic rings, F-18 radiochemistry was explored to develop an F-18 labeled [¹⁸F]FS1P1. If preclinical animal studies of [¹⁸F]FS1P1 demonstrated similar in vivo binding specificity with no defluorination, then [¹⁸F]FS1P1 could be a promising S1P1 radiotracer that could be translated to human clinical investigations using the toxicology data that has already been generated for [¹¹C]CS1P1. This plus the additional human experience with [¹¹C]CS1P1 from human studies, should facilitate implementation of human studies with [¹⁸F]FS1P1.

A multiple step, reliable strategy to synthesize [¹⁸F]FS1P1 with good yield and high quality for in vivo study was developed. Using the tertiary amine, TMEDA as an additive for the NO₂/[¹⁸F]F⁻ replacement reaction at optimal conditions, yielded the key intermediate ortho-[¹⁸F]fuorobenzaldehyde [¹⁸F]12 with 70% radioactive TLC yield starting with a reasonable amount of precursor 10 (4 mg). After that, [¹⁸F]12 went through continuous twice reductive amination reactions, followed by hydrolysis and neutralization, [¹⁸F]FS1P1 was achieved with good radiochemical yield (30-50%), >95% radiochemical and chemical purities and high molar activity (37-166.5 GBq/μmol, EOS). This suggested [¹⁸F]FS1P1 synthesized by the multiple step strategy is suitable for in vivo evaluation in animals.

In this study, the radiosynthesis of [¹⁸F]FS1P1 was accomplished from the substituted ortho-nitro benzenaldehyde precursor 10 via a multiple step procedure with high radiochemical yield and good quality. [¹⁸F]FS1P1 has a high possibility to be a promising F-18 radiotracer for imaging of S1P1 expression in response to neuroinflammation and other inflammatory diseases in vivo. Further translational clinical investigation of [¹⁸F]FS1P1 will confirm its suitability for human use.

Together, the studies of this disclosure suggest that [¹⁸F]FS1P1 has almost identical in vivo pharmacological properties as [¹¹C]CS1P1. The reliable multiple-step procedure of producing [¹⁸F]FS1P1 with good F-18 radiochemistry yield allows sufficient doses of [¹⁸F]FS1P1 for multiple PET studies. The data suggest that [¹⁸F]FS1P1 is a promising F-18 radiotracer for imaging S1P1 in vivo for inflammatory diseases.

### Example 4: Biodistribution Study of [¹⁸F]TZ33-21 ([18F]FS1P1) in Rats

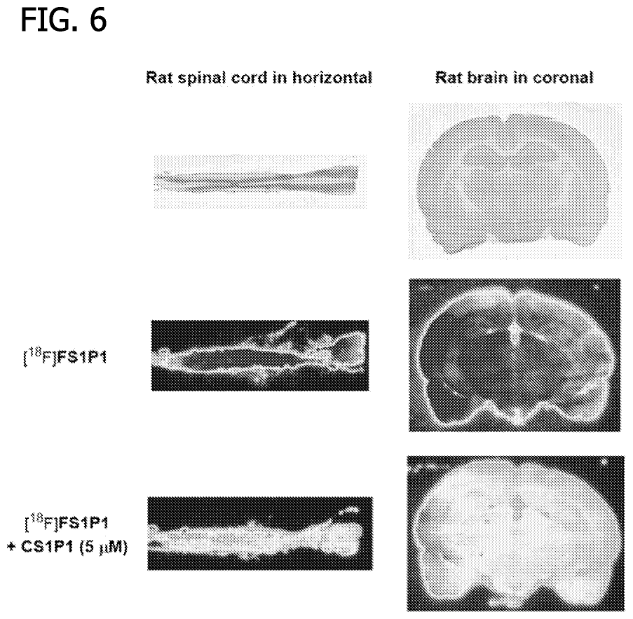

In vitro autoradiography (ARG) was conducted on rat spinal cord and brain section incubated with [¹⁸F]FS1P1, or [¹⁸F]FS1P1 and CS1P1 in blocking study (FIG. 6).

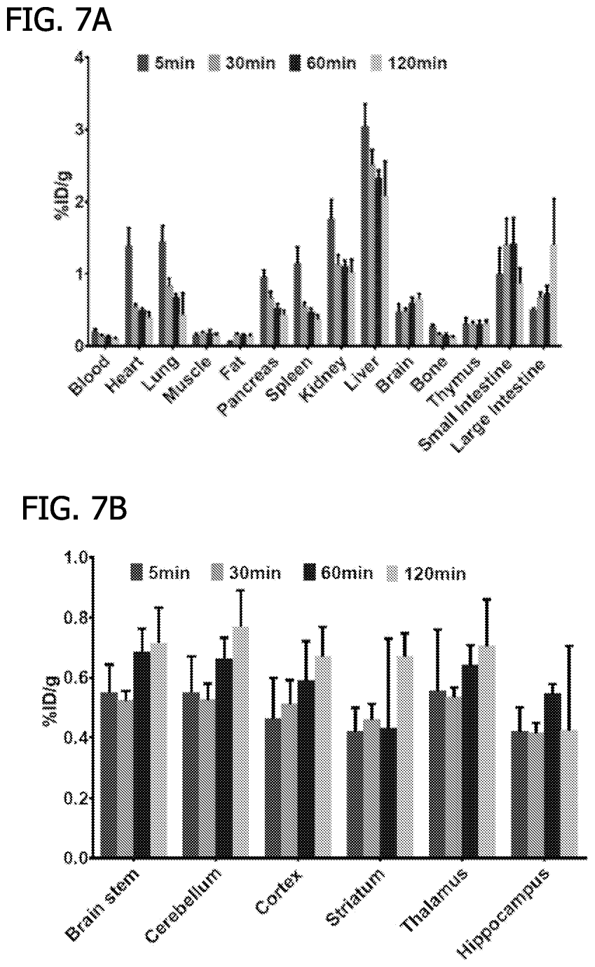

To evaluate the kinetics and the tissue distribution of [¹⁸F]FS1P1 in rodents, Sprague Dawley (SD) male rats (6-7 weeks old; 200-300 g) were used and euthanized at 5, 30, 60, and 120 min post-injection. The radioactivity uptake of each organ was calculated as percentage injected dose per gram (% ID/gram). As shown in FIG. 7A-FIG. 7B, the initial tracer uptake was high in most tissues at 5 min. The liver had the highest uptake (% ID/g) at 3.04±0.18, whereas the heart, lung, spleen, kidney, and small intestine had moderate to high uptake at 1.39±0.15, 1.44±0.13, 1.14±0.13, 1.76±0.15, and 1.00±0.21 respectively. The radioactivity was rapidly washed out from heart, lung, pancreas, spleen, kidney, and liver, at 30 min post injection with ID %/g values decreasing to 0.55±0.02, 0.84±0.05, 0.66±0.04, 0.54±0.03, 1.13±0.66, and 2.51±0.10 respectively as shown in FIG. 7A. Importantly, the bone uptake was relatively low with a % ID/g value of 0.27±0.03 at 5 min, and no significant change was observed from 5 min to 120 min (0.11±0.02), indicating no defluorination of [¹⁸F]FS1P1 occurred in vivo. Similar to [¹¹C]CS1P1, a high rat brain uptake of [¹⁸F]FS1P1 was observed as 0.48±0.06, 0.48±0.02, 0.60±0.04, and 0.65±0.03 at 5, 30, 60, and 120 min respectively, indicating the brain retention of [¹⁸F]FS1P1 were relatively stable. The uptake in the brain regions of interest including the brain stem, cerebellum, cortex, striatum, thalamus, and tissue distribution data suggested that [¹⁸F]FS1P1 has a high rat brain uptake and no defluorination happened in vivo.

Biodistribution study indicated that [¹⁸F]FS1P1 had a good brain uptake up to 2˜4% ID/gram from 5 to 120 min. No defluorination was observed, evidenced by no increase of the bone uptake. From the ARG study, [¹⁸F]FS1P1 exhibited strong signal, which was blocked in the presence of S1P1 antagonist CS1P1 (5 □M).

TABLE 3
Biodistribution of [¹⁸F]FS1P1 at 5,
30, 60, and 120 min post-injection in SD rats

| | Tissue uptake (% ID/g) | | | |
|---|---|---|---|---|
| Organs | 5 min | 30 min | 60 min | 120 min |
| Blood | 0.16 ± 0.09 | 0.13 ± 0.01 | 0.13 ± 0.01 | 0.10 ± 0.01 |
| Heart | 1.02 ± 0.69 | 0.54 ± 0.03 | 0.49 ± 0.02 | 0.39 ± 0.07 |
| Lung | 1.08 ± 0.74 | 0.84 ± 0.09 | 0.68 ± 0.04 | 0.43 ± 0.30 |
| Muscle | 0.11 ± 0.07 | 0.19 ± 0.01 | 0.18 ± 0.05 | 0.15 ± 0.02 |
| Fat | 0.05 ± 0.03 | 0.16 ± 0.02 | 0.15 ± 0.02 | 0.14 ± 0.02 |
| Pancreas | 0.72 ± 0.47 | 0.66 ± 0.08 | 0.52 ± 0.05 | 0.43 ± 0.06 |
| Spleen | 0.86 ± 0.58 | 0.54 ± 0.06 | 0.47 ± 0.05 | 0.37 ± 0.05 |
| Kidney | 1.33 ± 0.87 | 1.13 ± 0.13 | 1.10 ± 0.08 | 1.02 ± 0.17 |
| Liver | 2.30 ± 1.50 | 2.51 ± 0.20 | 2.32 ± 0.10 | 2.08 ± 0.47 |
| Bone | 0.20 ± 0.13 | 0.16 ± 0.01 | 0.15 ± 0.02 | 0.11 ± 0.02 |
| Thymus | 0.23 ± 0.16 | 0.30 ± 0.03 | 0.30 ± 0.04 | 0.31 ± 0.05 |
| Small intestine | 0.75 ± 0.57 | 1.40 ± 0.36 | 1.42 ± 0.36 | 0.87 ± 0.21 |
| Brain stem | 0.48 ± 0.14 | 0.52 ± 0.03 | 0.68 ± 0.07 | 0.71 ± 0.11 |
| Cerebellum | 0.42 ± 0.28 | 0.52 ± 0.05 | 0.66 ± 0.07 | 0.77 ± 0.12 |
| Cortex | 0.35 ± 0.25 | 0.51 ± 0.07 | 0.59 ± 0.13 | 0.67 ± 0.10 |
| Striatum | 0.32 ± 0.21 | 0.46 ± 0.05 | 0.43 ± 0.29 | 0.67 ± 0.07 |
| thalamus | 0.42 ± 0.31 | 0.53 ± 0.03 | 0.64 ± 0.06 | 0.70 ± 0.15 |
| Hippocampus | 0.32 ± 0.21 | 0.41 ± 0.03 | 0.54 ± 0.03 | 0.42 ± 0.28 |
| total brain | 2.65 ± 1.63 | 3.41 ± 0.29 | 4.12 ± 0.70 | 4.58 ± 0.92 |

Additional methods for the rodent experiments are described below.

All animal experiments were conducted under protocols approved by Washington University's Institutional Animal Care and Use Committee (IACUC). Rodent studies were conducted at Preclinical Imaging Facility at the Washington University School of Medicine.

### Ex Vivo Biodistribution Study in Sprague Dawley Rats

A dose of [¹⁸F]FS1P1 (˜3.7 MBq/100 μL) was injected via the tail vein into SD rats (male; 6-7 weeks old; 200-300 g). Rats were euthanized under anesthesia at 5, 30, 60, and 120 min post-injection (n=4 per group). Tissues of interest including blood, heart, lung, muscle, fat, pancreas, spleen, kidney, liver, brain, bone, thymus, small intestine, and large intestine were collected, weighed, and counted on an automated Beckman Gamma counter (Beckman, Brea, CA). To evaluate the uptake of [¹⁸F]FS1P1 within the brain, brain dissection was performed and different brain regions including brain stem, cerebellum, cortex, striatum, thalamus, and hippocampus were collected and evaluated. The uptake of each organ was calculated and expressed as a percentage of the injection dose per gram of wet tissue (% ID per gram).

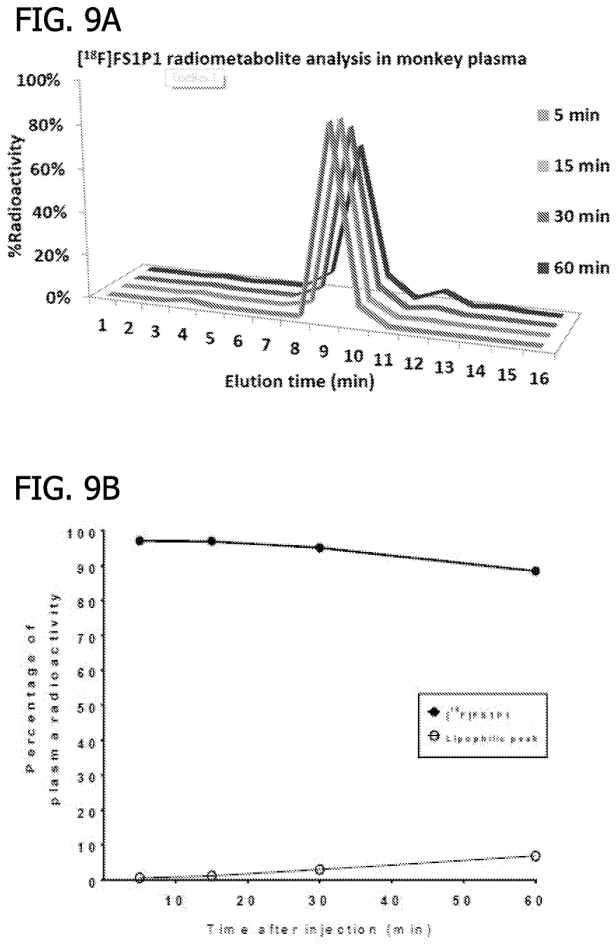

The tissue distribution study in Sprague-Dawley rats showed that [¹⁸F]FS1P1 has good brain uptake comparable to [11C]CS1P1 and similar uptake in other tissues. For the brain regions of interests, [¹⁸F]FS1P1 also showed relatively high uptake and good retention, indicating good BBB permeability of [¹⁸F]FS1P1. This is consistent with its experimental measure of Log D_{7.4} value (2.62±0.31), considered within the optimal range of 1 to 3 for most CNS drugs. Importantly, a very low bone uptake was observed and has no increasing trend from 5 to 120 min post-injection, indicating [¹⁸F]FS1P1 has no defluorination in vivo, which is a critical concern for most F-18 radiotracers. The microPET study indicates that [¹⁸F]FS1P1 and [¹¹C]CS1P1 have similar pharmacokinetics in the nonhuman primate brain, as well as a similar distribution in the brain regions of interest as shown in FIG. 9B. The brain uptake of [¹⁸F]FS1P1 in different macaques also showed good brain time-activity curve reproducibility. PET studies with different injection doses of 8.13 MBq (2.17 mCi) versus 25.2 MBq (6.79 mCi) led to similar visualization of [¹⁸F]FS1P1 in the brain with high resolution. The brain time-activity curves of tracer uptake (SUV) were almost identical, suggesting the lower dose of [¹⁸F]FS1P1 could be used in future studies. The metabolite analysis of nonhuman primate plasma samples showed no major radiometabolite were formed within 60 min post-injection of [¹⁸F]FS1P1, eliminating the concern that potential radioactive metabolites may confound PET quantification of brain uptake. Since different species may have different metabolism, human plasma radiometabolite analysis is needed to confirm no newly formed radiometabolite will impact precise PET with [¹⁸F]FS1P1 measurement in human beings.

### Example 5: Biodistribution Studies of [¹⁸F]TZ33-21 ([18F]FS1P1) in Non-Human Primates (NHPs)

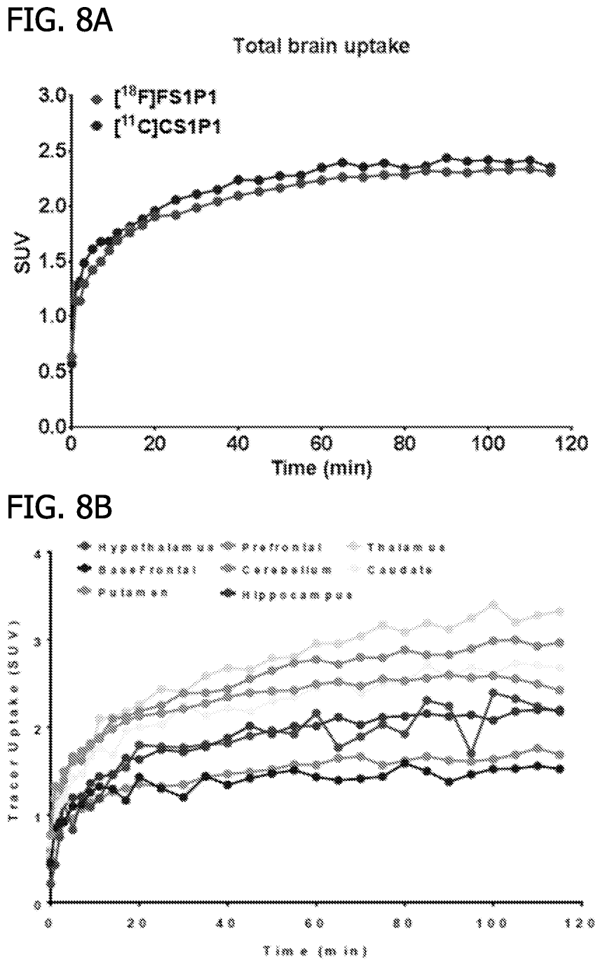

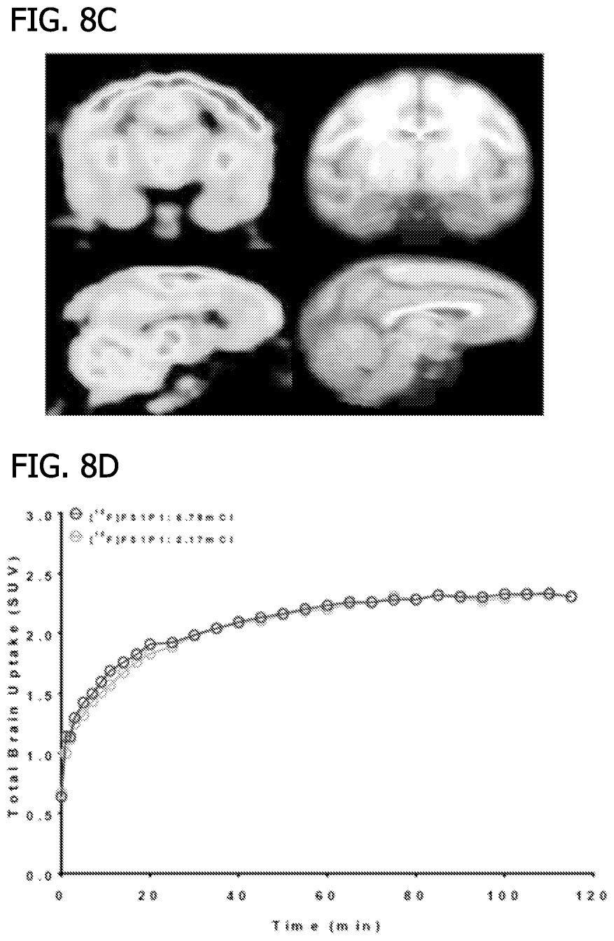

To further confirm if [¹⁸F]FS1P1 is suitable for PET imaging study of S1P1 in the brain, microPET studies in the brain of a male cynomolgus macaque was performed. The microPET brain imaging scans were carried out in the same animal to precisely compare the pharmacokinetics of [¹⁸F]FS1P1 and [¹¹C]CS1P1, which shared the same chemical structure, but were labeled with different isotope. As shown in FIG. 8A, the time tissue activity curves of the brain uptake (standard uptake value, SUV) of [¹⁸F]FS1P1 and [¹¹C]CS1P1 were almost identical. Both [¹⁸F]FS1P1 and [¹¹C]CS1P1 penetrated the BBB very well and the brain uptake reached a maximum SUV value of ˜2.3 from 20 to 120 min post-injection, indicating [¹⁸F]FS1P1 and [¹¹C] have identical pharmacokinetics in the macaque brain.

Among different brain regions, [¹⁸F]FS1P1 showed a high uptake in thalamus, putamen, caudate, and prefrontal cortex, whereas hippocampus were 0.55±0.06, 0.55±0.07, 0.47±0.08, 0.42±0.05, 0.56±0.12, and 0.42±0.05 at 5 min respectively; and a slight increase was observed from 5 min to 120 min as shown in FIG. 7B. The cerebellum and basal frontal cortex had only moderately high uptake (FIG. 8B and 8C). In addition, the initial studies of [¹⁸F]FS1P1 in the brains of different macaques indicated a good reproducibility, the high and low injection doses of ˜8.13 MBq (2.17 mCi) and ˜25.2 MBq (6.79 mCi) injection provided similarly good quality visualization of brain and almost identical time tissue activity curves as shown in FIG. 8D. Further PET imaging modeling and binding potential studies will determine the optimal dose of [¹⁸F]FS1P1 for particular applications.

Using a Focus 220 microPET scanner to collect dynamic scans for 2 hours after intravenous injection of ˜0.35 GBq dose into male macaques. Radiometabolites were measured from arterial blood samples collected at 5, 15, 30, and 60 min post-injection of radiotracer. The HPLC eluent was collected, and fractions were counted in a gamma counter.

HPLC radiometabolism analysis of macaque plasma samples collected at different time points post-injection of [¹⁸F]FS1P1 permitted analysis of the stability of [¹⁸F]FS1P1 and the radiometabolites in vivo that can be detected. As shown in FIG. 9A, the parent radioactive compound [¹⁸F]FS1P1 was the only major radioactive peak with a retention time of ˜9 min on the HPLC, the percentage of the parental radiotracer [¹⁸F]FS1P1 was 96%, 96%, 94%, and 88% of total radioactivity at 5, 15, 30, and 60 min post-injection, respectively (FIGS. 9A and 9B). Nonobvious radiometabolite peak was detected in the 5 and 15 min plasma samples. For 30 and 60 min plasma samples, only a negligible lipophilic radioactive peak with a retention time of ˜12 min, was observed with 2% and 6% of the total radioactivity respectively (FIGS. 9A and 9B). No hydrophobic radioactive peak was detected for all plasma samples collected from 5, 15, 30, and 60 min post-injection of [¹⁸F]FS1P1, suggesting [¹⁸F]FS1P1 has favorable in vivo stability, and no major radiometabolite will confound the PET with [¹⁸F]FS1P1 measurement of the S1P1 in the brain.

The biodistribution studies indicate that [¹⁸F]FS1P1 had good brain uptake without in vivo defluorination. The PET brain studies in macaques indicated that [¹⁸F]FS1P1 had a good brain uptake and similar pharmacokinetics with [¹¹C]CS1P1 in macaque. The initial radiometabolite analysis of [¹⁸F]FS1P1 in macaque indicated [¹⁸F]FS1P1 has a good in vivo stability with no major radiometabolite emerged in vivo within 60 min post-injection. [¹⁸F]FS1P1 could be a promising S1P1 radiotracer for investigating neuroinflammation as well as other inflammatory diseases.

Additional methods of the non-human primate experiments are described below.

All animal experiments were conducted under protocols approved by Washington University's Institutional Animal Care and Use Committee (IACUC). The NHP study was conducted in the NHP microPET facility at the Washington University School of Medicine.

### MicroPET Brain Study in Cynomolgus Macaque

Male macaques (˜10 kg) were used for PET imaging data acquisition with a microPET Focus 220 scanner (Siemens Inc., Knoxville, TN). Animals were anesthetized using ketamine and glycopyrrolate and maintained with inhalation of isoflurane. Core temperature was kept at 37° C. with a heated water blanket. The head was secured in a customized head holder. Subsequently, a 2 h dynamic PET scan was performed after administration of radiotracers (8.13 MBq (2.17 mCi) or ˜25.2 MBq (6.79 mCi)) via the venous catheter. PET scans data were collected from 0-120 min with the following time frames: 3×1 min, 4×2 min, 3×3 min, and 20×5 min. Emission data were corrected for dead time, scatter, and attenuation and then reconstructed. For quantitative analyses, dynamic PET images were co-registered to a standardized monkey MRI template using PMOD software 4.02 (PMOD Technologies, Zürich, Switzerland) (H. Jiang, et al., ACS Chem. Neurosci., 2021, 12, 3733-3744.). Predefined brain regions of interest from the template were applied to the co-registered PET image to obtain regional time-activity curves. The measurement of the brain uptake of radiotracer was standardized to body weight and the dose of radioactivity injected to yield a standardized uptake value (SUV).

### Radiometabolite Analysis of Nonhuman Primate Plasma Samples

HPLC radiometabolite analysis of the macaque plasma samples collected at different time points post-injection of [¹⁸F]FS1P1 was performed as previously reported (H. Liu, et al., Mol. Imaging Biol., 2020, 22, 1362-1369.). A male macaque (˜10 kg) was intravenously injected with ˜0.35 GBq of [¹⁸F]FS1P1. Arterial blood samples (˜1.5 mL) were collected using heparinized syringes at 5, 15, 30, and 60 min post-injection. Plasma (400 μL) was then collected and mixed with 1.2 mL ice-cold acetonitrile to deproteinize. After centrifuge, 200 μL of the supernatant was loaded onto an analytical HPLC system with an SB C-18 analytical HPLC column (Agilent Technologies, Santa Clara, CA) and eluted with acetonitrile/0.1 M ammonium formate buffer pH 4.5, (75/25, v/v) with a flow rate of 1.13 mL/min. The eluted fractions were collected at one-minute intervals for a total of 16 minutes, the radioactivity of each fraction collection was counted in an automated gamma counter (Beckman, Brea, CA), and results were corrected by background radioactive counts and physical decay. The chromatography was regenerated represented by the percentage that was calculated by radioactivity count of every minute collection divided by the total injection amount on the HPLC system and multiplied by 100.

### Example 6: Human Biodistribution Study of [¹¹C]CS1P1

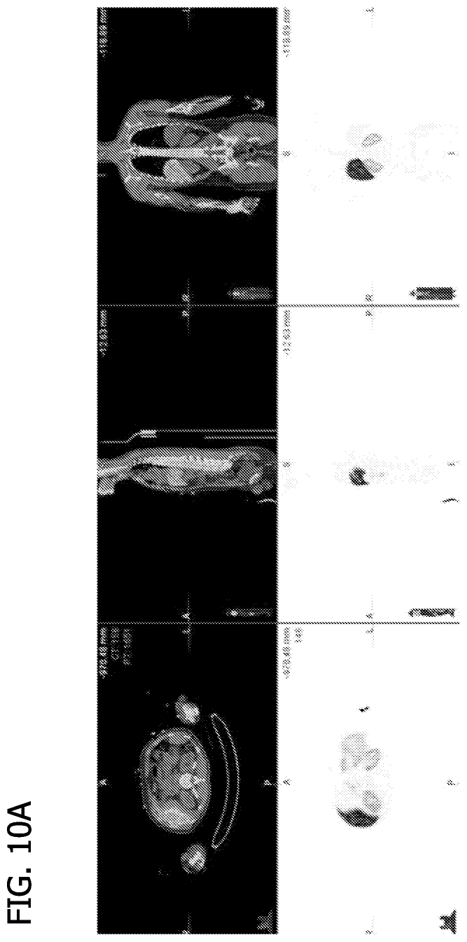

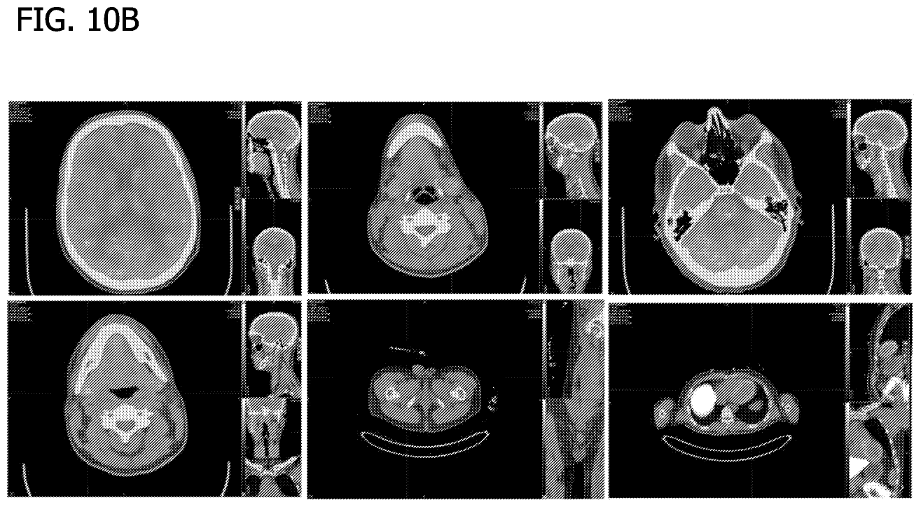

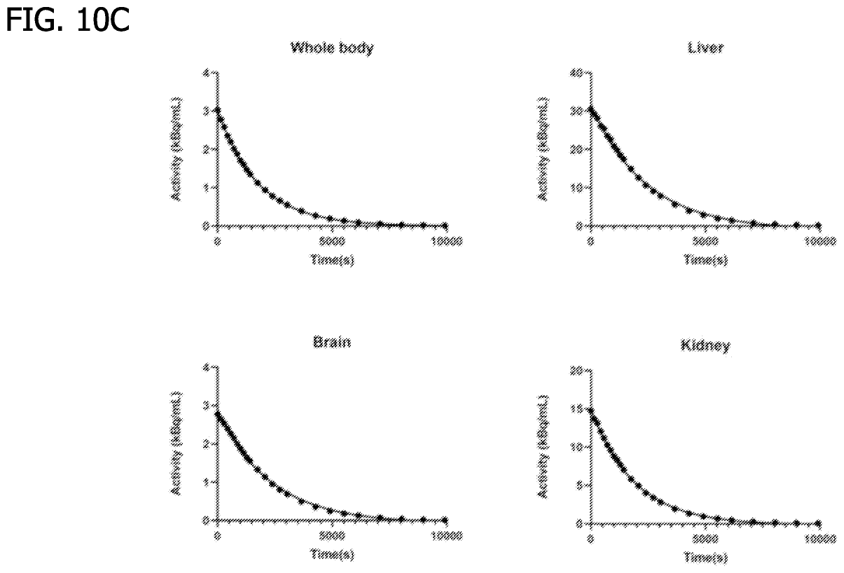

One human male (88 kg, 28 years old, SIEMENS, patient ID: 1179101) was imaged with a dose of 7.2 mCi (FIG. 10A). Good uptake was found in the brain (FIG. 10B). Residence time and total absorbed dose in individual organs was also measured (FIG. 10C and Table 4).

TABLE 4
Residence time and total absorbed dose of source organs
determined from a single healthy participant

| Organ | Residence time (h) | μGy/MBq |
|---|---|---|
| Brain | 0.00434 ± 0.00072 | 1.02 ± 0.1 |
| Gall bladder | 0.00044 ± 0.00029 | 4.82 ± 0.19 |
| Heart wall | 0.00148 ± 0.00021 | 2.06 ± 0.13 |
| Kidneys | 0.00525 ± 0.00024 | 4.66 ± 0.22 |
| Liver | 0,06418 ± 0.00802 | 9.88 ± 0.03 |
| Lung | 0.00680 ± 0.00128 | 2.13 ± 0.56 |
| Pancreas | 0.00026 | 2.22 ± 0.07 |
| Spleen | 0.00195 ± 0.00057 | 3.09 ± 0.35 |
| Urinary bladder | 0.00030 ± 0.00011 | 0.48 ± 0.32 |
| Effective does (μSv/MBq) | | 1.60 ± 0.15 |

Thus, imaging with [¹¹C]CS1P1 for clinical PET studies is safe. Administration of 10 mCi (370 MBq) of [¹¹C]CS1P1 resulted in a total human effective dose of <0.6 mSv and thus allow for multiple PET scans of the same subject per year.

### Example 7: Additional [¹⁸F]TZ33-21 ([18F]FS1P1) Derivatives and Binding Data

The following are additional related compounds:

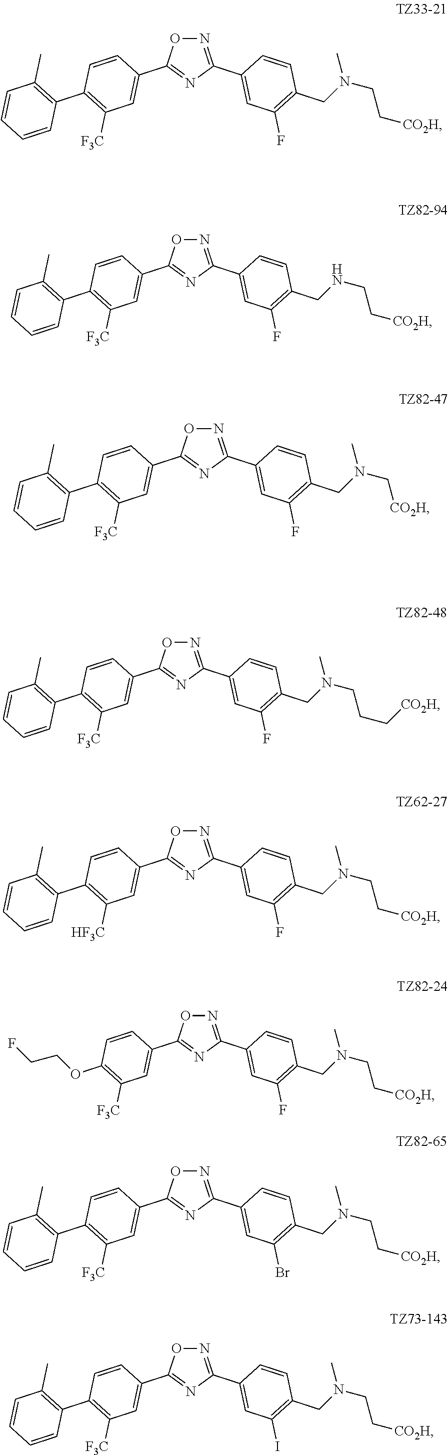

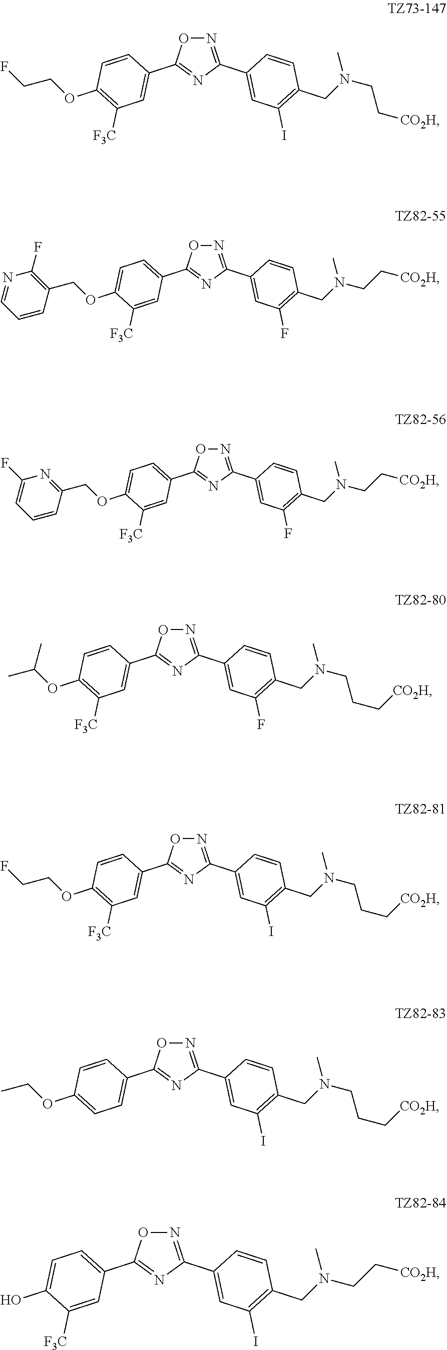

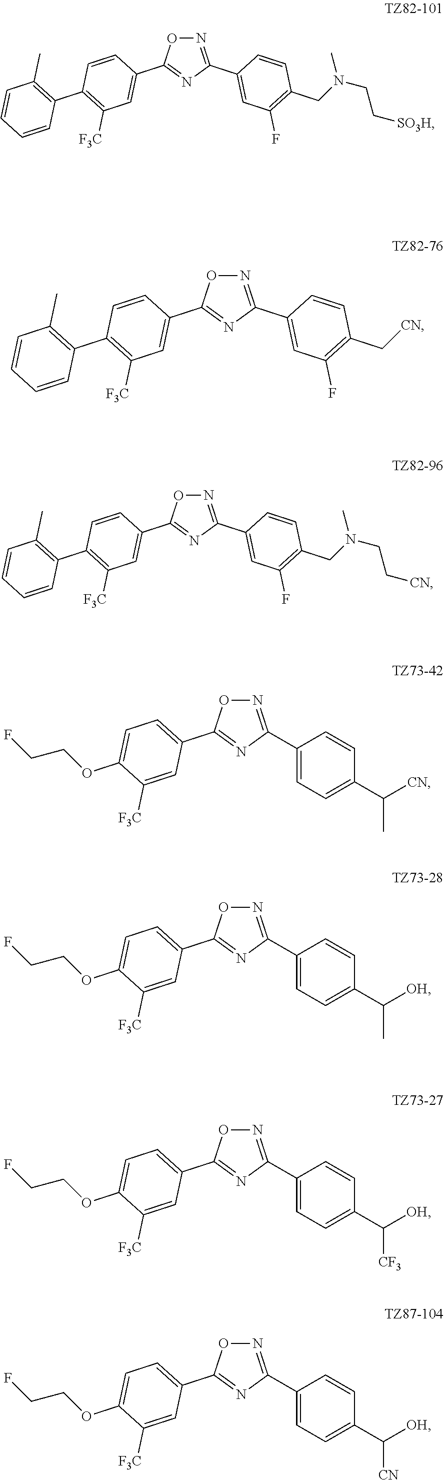

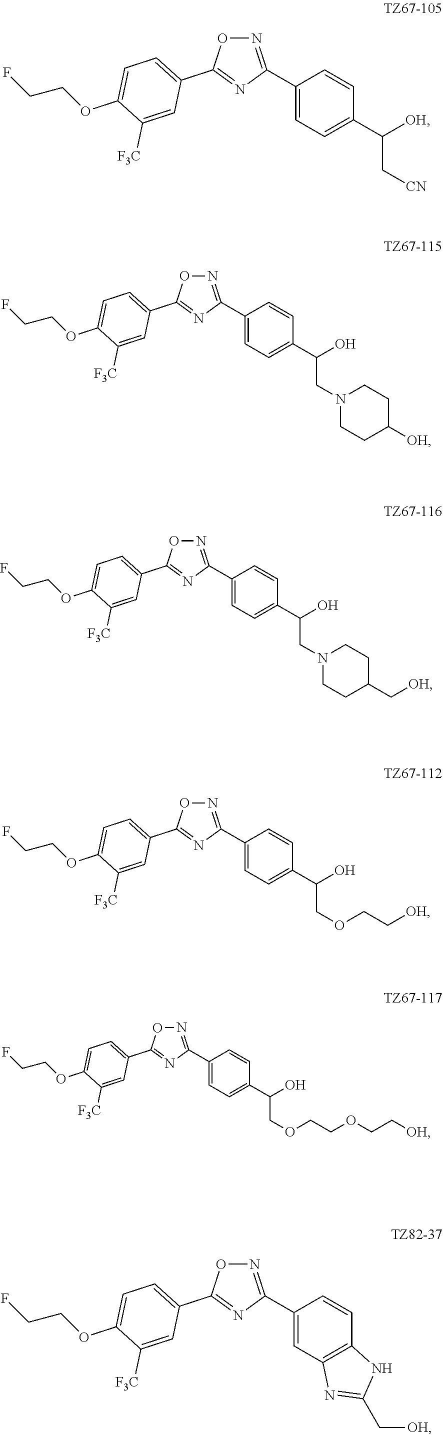 [SMILES: N#CCC(O)c1ccc(-c2noc(-c3ccc(OCCF)c(C(F)(F)F)c3)n2)cc1 ; OC1CCN(CC(O)c2ccc(-c3noc(-c4ccc(OCCF)c(C(F)(F)F)c4)n3)cc2)CC1 ; OCC1CCN(CC(O)c2ccc(-c3noc(-c4ccc(OCCF)c(C(F)(F)F)c4)n3)cc2)CC1 ; OCCOCC(O)c1ccc(-c2noc(-c3ccc(OCCF)c(C(F)(F)F)c3)n2)cc1 ; OCCOCCOCC(O)c1ccc(-c2noc(-c3ccc(OCCF)c(C(F)(F)F)c3)n2)cc1 ; OCc1nc2cc(-c3noc(-c4ccc(OCCF)c(C(F)(F)F)c4)n3)ccc2[nH]1]

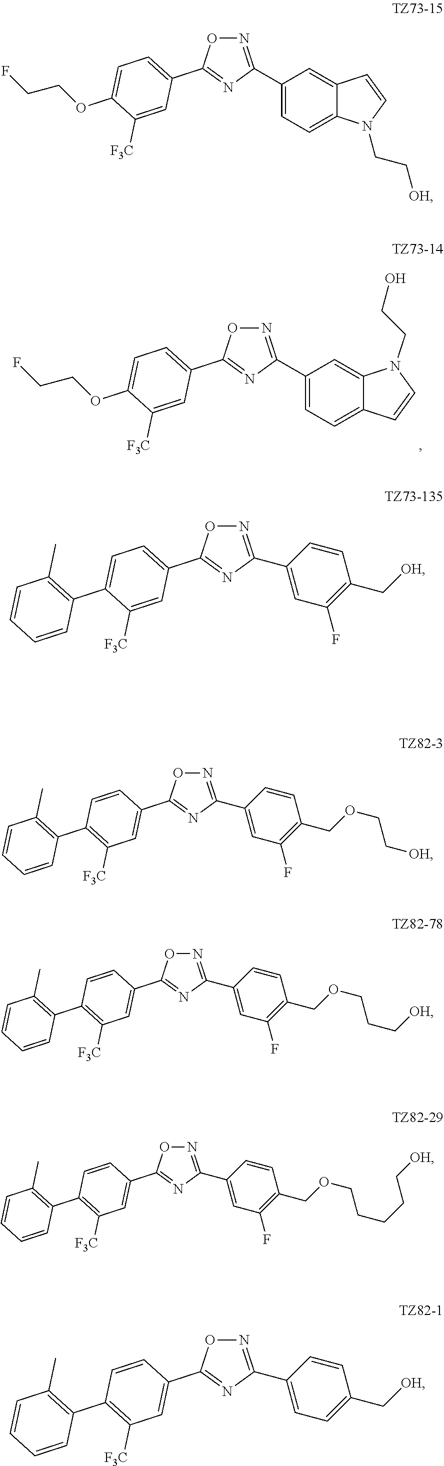

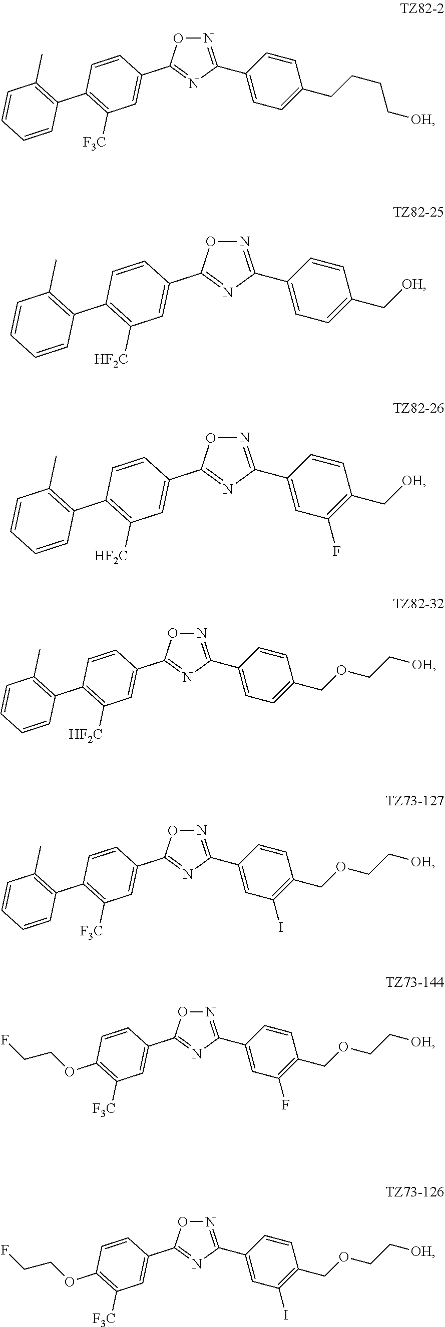

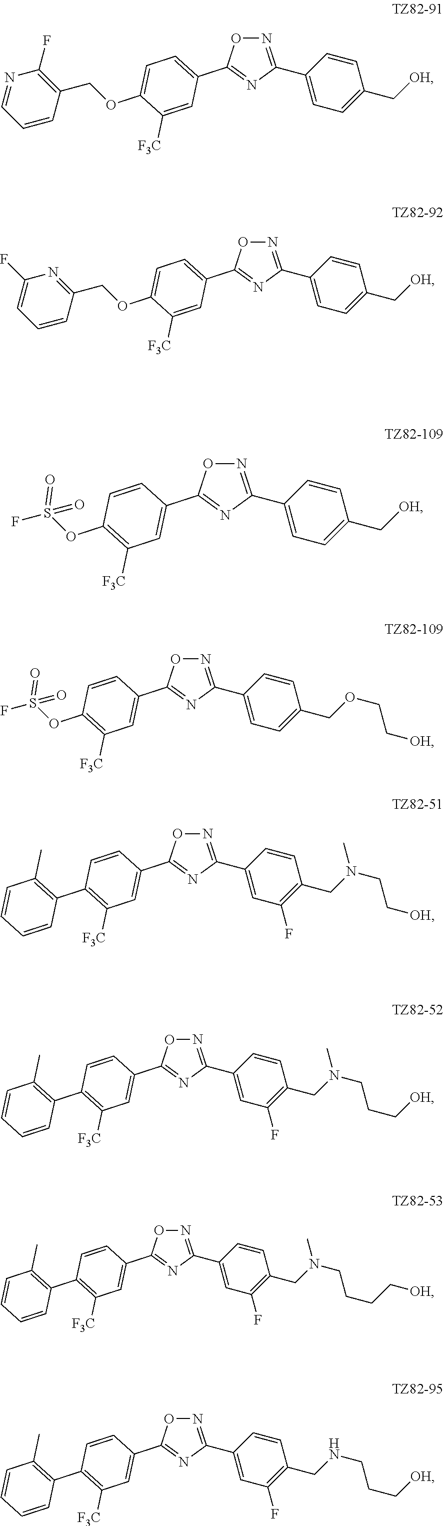

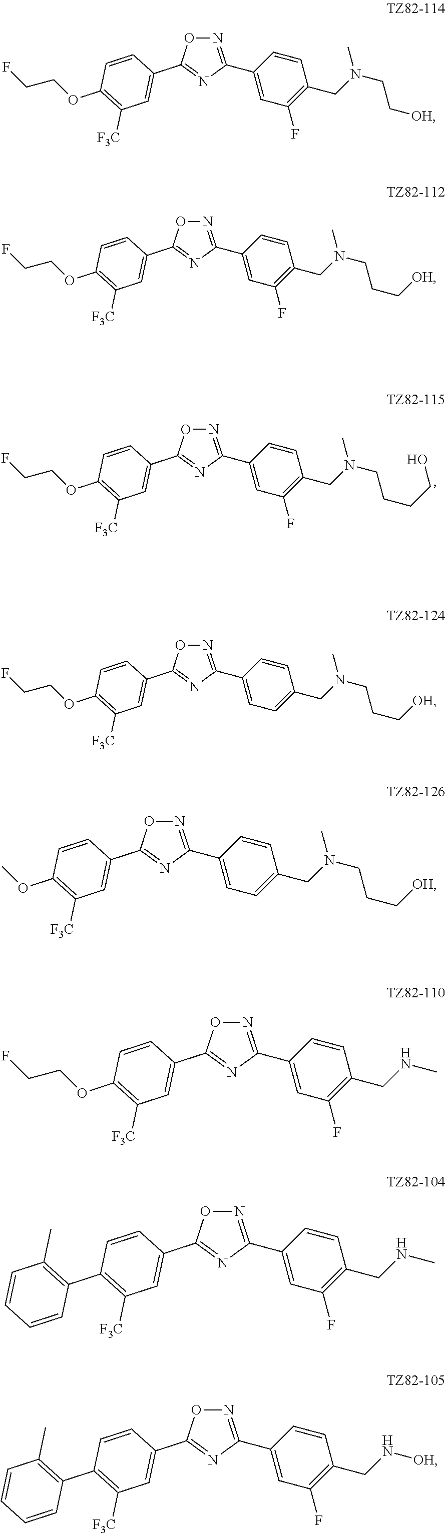

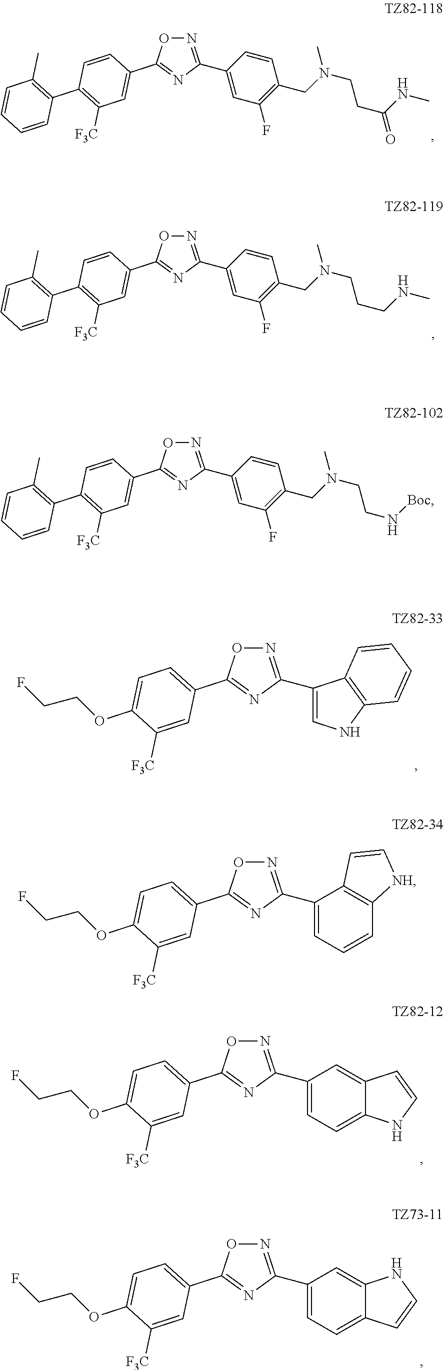

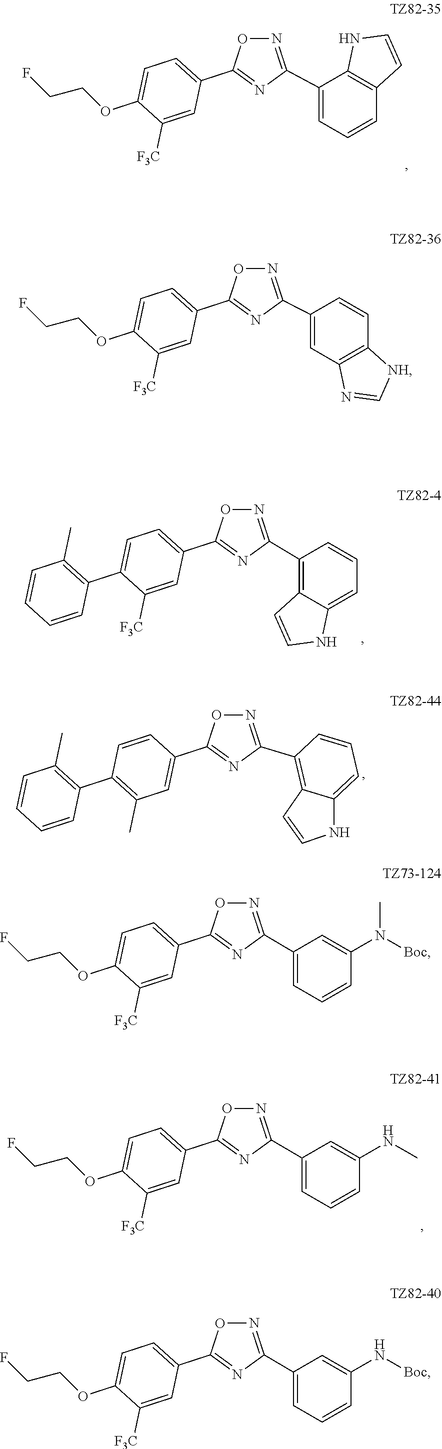

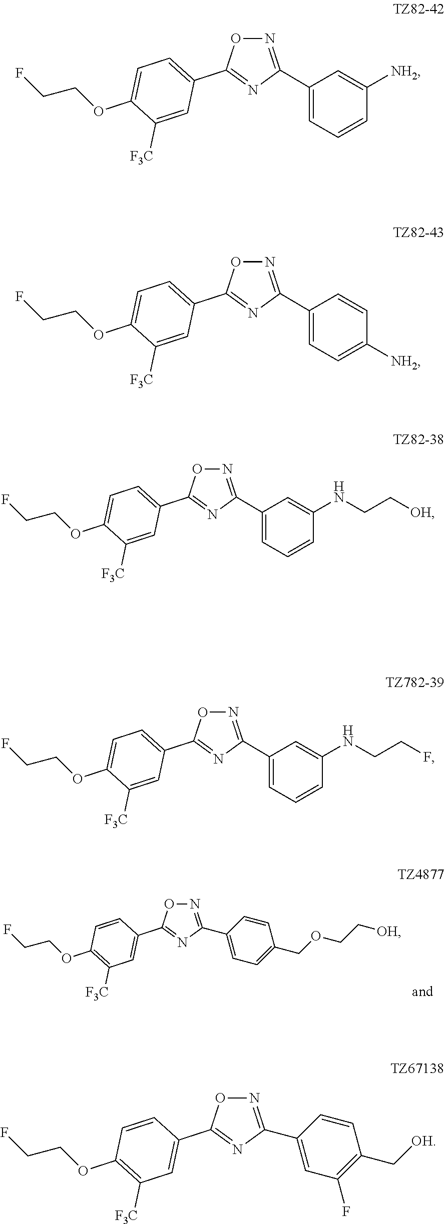 [SMILES: Nc1cccc(-c2noc(-c3ccc(OCCF)c(C(F)(F)F)c3)n2)c1 ; Nc1ccc(-c2noc(-c3ccc(OCCF)c(C(F)(F)F)c3)n2)cc1 ; OCCNc1cccc(-c2noc(-c3ccc(OCCF)c(C(F)(F)F)c3)n2)c1 ; FCCNc1cccc(-c2noc(-c3ccc(OCCF)c(C(F)(F)F)c3)n2)c1 ; OCCOCc1ccc(-c2noc(-c3ccc(OCCF)c(C(F)(F)F)c3)n2)cc1 ; OCc1ccc(-c2noc(-c3ccc(OCCF)c(C(F)(F)F)c3)n2)cc1F]

These compounds were tested for binding potencies. The results are shown in Tables 5-7 below.

TABLE 5
Binding potencies of S1P1 Compounds

| Com- | IC₅₀ (nM) | | | | | |
|---|---|---|---|---|---|---|
| pound | S1P1 | S1P2 | S1P3 | S1P4 | S1P5 | Published |
| S1P | 1.4 ± 0.3 | 3.6 ± | 0.4 ± | 151 ± | 3.1 ± 1.1 | Eur. J. Med. |
| | | 0.5 | 0.2 | 82 | | Chem. |
| | | | | | | 2018, 150, |
| | | | | | | 796-808 |
| TZ33-21 | 2.1 ± 1.6 | >1000 | >1000 | >1000 | 6.7 ± 2.9 | |
| TZ82-94 | 0.7 | — | — | — | 7.2 | |
| TZ82-47 | 7.6 | — | — | — | 77.3 | |
| T1Z82-48 | 0.8 | — | — | — | 23.1 | |
| TZ82-27 | 8.4 | — | — | — | 10.5 | |
| TZ82-24 | 7.6 ± 6.0 | — | — | — | 1.9 | |
| TZ82-65 | 2.1 | — | — | — | 11.0 | |
| TZ73-143 | 5.2 ± 2.7 | — | — | — | — | |
| TZ73-147 | 33.1 ± 11.1 | — | — | — | — | |
| TZ82-55 | 158.9 | — | — | — | 74.4 | |
| TZ82-56 | 55.4 | — | — | — | 39.3 | |
| TZ82-80 | — | — | — | — | — | |
| TZ82-81 | — | — | — | — | — | |
| TZ82-83 | — | — | — | — | — | |
| TZ82-84 | — | — | — | — | — | |
| TZ82-101 | 2.3 | — | — | — | — | |
| TZ82-34 | 4.3 ± 1.4 | — | — | — | — | |
| TZ73-12 | 11 ± 1.8 | — | — | — | — | |
| TZ73-11 | 34 ± 2.4 | — | — | — | — | |
| TZ82-35 | 98 ± 26 | — | — | — | — | |
| TZ82-36 | 57 ± 7.6 | — | — | — | — | |
| TZ82-37 | 28 ± 5.8 | — | — | — | — | |
| TZ82-4 | 1.1 ± 0.1 | — | — | — | 28.4 | |
| TZ82-44 | | — | — | — | — | |
| TZ73-15 | 57 ± 4.6 | — | — | — | — | |
| TZ73-14 | 157 ± 24 | — | — | — | — | |
| TZ73-38 | 108 ± 3.3 | — | — | — | — | |
| TZ73-39 | 235 ± 5.0 | — | — | — | — | |
| TZ73-124 | >1000 | — | — | — | — | |
| TZ82-41 | 82 ± 1.8 | — | — | — | — | |
| TZ82-40 | >1000 | — | — | — | — | |
| TZ82-42 | 29 ± 0.5 | — | — | — | — | |
| TZ82-43 | 44 ± 0.6 | — | — | — | — | |
| TZ82-76 | 1.4 | — | — | — | 148.7 | |
| TZ82-96 | 1.8 | — | — | — | 683.1 | |
| TZ82-102 | 23.8 | — | — | — | — | |
| TZ73-28 | 21 ± 2.5 | — | — | — | — | |
| TZ73-27 | 15 ± 2.6 | — | — | — | — | |
| TZ73-104 | 928 ± 280 | — | — | — | — | |
| TZ73-105 | 24.7 ± 5.0 | — | — | — | — | |
| TZ73-115 | 377 ± 277 | — | — | — | — | |
| TZ73-116 | 129 ± 47 | — | — | — | — | |
| TZ73-112 | 18.3 ± 3.0 | — | — | — | — | |
| TZ73-117 | 91 ± 27 | — | — | — | — | |
| TZ73-135 | 8.4 ± 2.1 | — | — | — | 75.0 | |
| TZ82-3 | 12.3 ± 6.6 | — | — | — | 186.3 | |
| TZ82-78 | 2.8 | — | — | — | 274.4 | |
| TZ82-29 | 4.9 | — | — | — | 380.7 | |
| TZ82-1 | 4.6 | — | — | — | 55.6 | |
| TZ82-2 | 9.8 | — | — | — | 764.3 | |
| TZ82-25 | | — | — | — | — | |
| TZ82-26 | 2.9 | — | — | — | 85.2 | |
| TZ82-32 | | — | — | — | — | |
| TZ73-127 | 66.3 | — | — | — | — | |
| TZ73-144 | 17.0 ± 7.0 | — | — | — | — | |
| TZ73-126 | 118 | — | — | — | — | |
| TZ82-91 | 238.7 | — | — | — | 182.5 | |
| TZ82-92 | 242.5 | — | — | — | 934.0 | |
| TZ82-108 | 692.9 | — | — | — | — | |
| TZ82-109 | 207.0 | — | — | — | — | |
| TZ82-104 | 0.6 | — | — | — | — | |
| TZ82-105 | 8.7 | — | — | — | — | |
| TZ82-51 | 3.4 | — | — | — | 183.1 | |
| TZ82-52 | 5.8 | — | — | — | 239.0 | |
| TZ82-53 | 4.9 | — | — | — | 245.3 | |
| TZ82-95 | 2.1 | — | — | — | 12.8 | |
| TZ82-114 | 14.6 | — | — | — | 120 | |
| TZ82-112 | 15.8 | — | — | — | 234 | |
| TZ82-115 | — | — | — | — | 721 | |
| TZ82-124 | — | — | — | — | 450 | |
| TZ82-126 | — | — | — | — | 1942 | |
| TZ82-110 | 13.4 | — | — | — | — | |
| TZ82-118 | 3.0 | — | — | — | — | |
| TZ82-119 | — | — | — | — | — | |

TABLE 6
More binding potencies of S1P1 Compounds

| | | IC₅₀ for | IC₅₀ for | IC₅₀ for | IC₅₀ for |
|---|---|---|---|---|---|
| | IC₅₀ for | S1P2 | S1P3 | S1P4 | S1P5 |
| Compounds | S1P1 (nM) | (nM) | (nM) | (nM) | (nM) |
| 1Z82-112 | 15.8, 2.03, | >1000 | 746 | >1000 | >1000 |
| | 8.17 (8.7 ± | | | | |
| | 6.9) | | | | |
| 1Z82-24 | 7.3 | >1000 | >1000 | >1000 | 99.1 |
| 1Z82-47 | Not detected | >1000 | Not fit | >1000 | Not fit |
| 1Z82-48 | Not detected | >1000 | 189 | >1000 | 194.3 |
| 1Z82-51 | Not detected | >1000 | 454 | >1000 | >1000 |
| 1Z82-52 | Not detected | >1000 | 418 | >1000 | >1000 |

TABLE 7
Further binding potencies of S1P1 Compounds

IC50 (nM)

| Ligands | S1P1 | S1P2-5 |
|---|---|---|
| TZ48-77 | 14.0 ± 0.4 | >1000 |
| TZ67-138 | 8.6 ± 0.9 | >1000 |
| TZ82-34 | 4.3 ± 1.4 | >1000 |
| TZ33-21 | 2.1 ± 1.6 | >1000 |
| TZ82-47 | 7.6 ± 0.2 | >1000 |
| TZ82-48 | 0.8 ± 0.2 | >1000 |
| TZ73-135 | 8.4 ± 0.2 | >1000 |
| TZ82-3 | 12.3 ± 0.5 | >1000 |
| TZ82-29 | 16.1 ± 0.3 | >1000 |

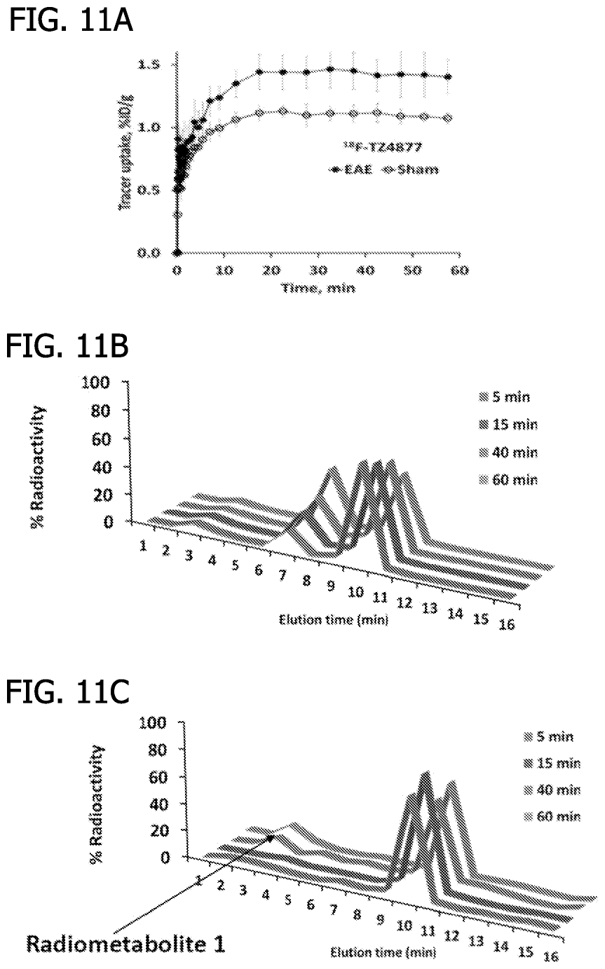

### Example 8: Biodistribution Studies of [¹⁸F]TZ4877

[¹⁸F]TZ4877 was tested for radiometabolite analysis of rate plasma and rat brain samples (FIG. 11A, FIG. 11B, FIG. 11C). [¹⁸F]TZ4877 has IC₅₀ of 14.01±0.01 nM for S1P1 and IC₅₀>1000 for S1P2-5. The structure of [¹⁸F]TZ4877 is shown below:

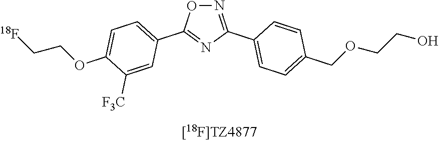 [SMILES: OCCOCc1ccc(-c2noc(-c3ccc(OCC[18F])c(C(F)(F)F)c3)n2)cc1]

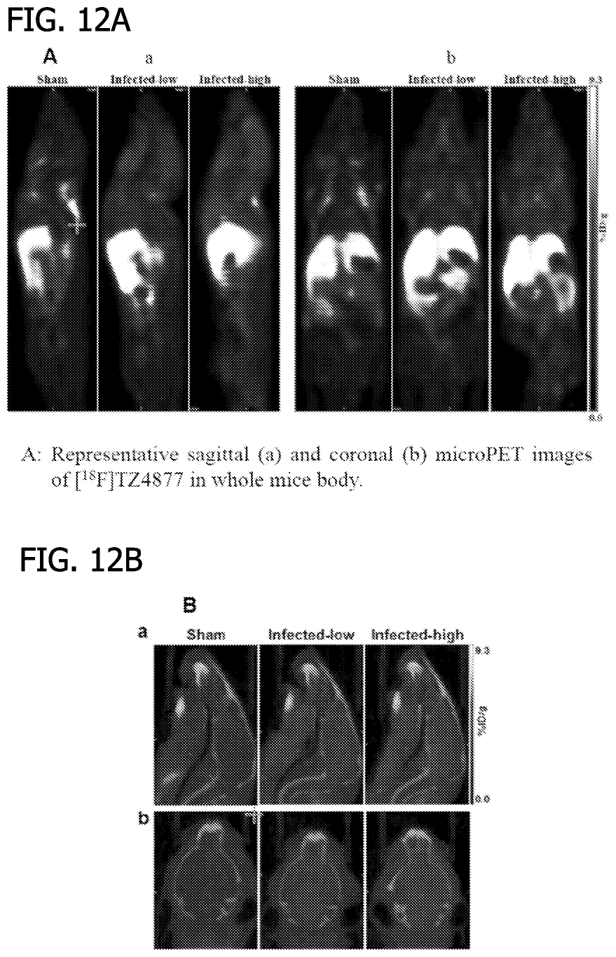

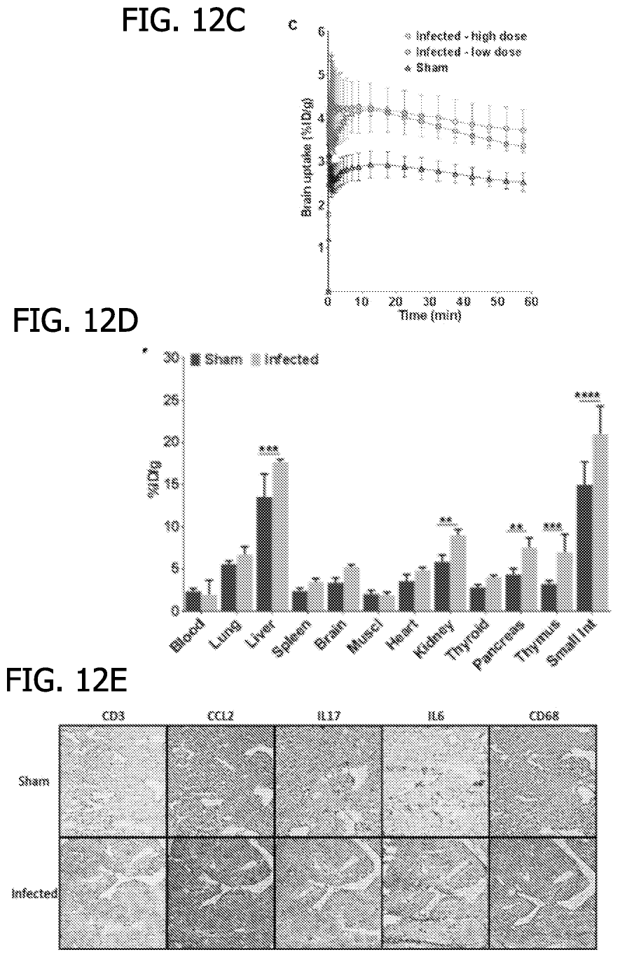

The potential application of a S1P1 radiotracer for peripheral neuroinflammation was also examined by studying S1P1 expression in S. aureus infection in mice using PET with [¹⁸F]TZ4877. microPET images were taken of [¹⁸F]TZ4877 in whole mice bodies (FIG. 12A) and brains (FIG. 12B) with no infection, low infection, and high infection. Brain uptake was quantified, with higher uptake in infected mice (FIG. 12C).

Mice were euthanized at 30 min, and S. aureus was injected 24 hours prior to radiotracer injection. Tissue uptake of [¹⁸F]TZ4877 was quantified (FIG. 12D). IHC staining was used for confirmation in infected versus sham controls (FIG. 12E). Increasing signals of the inflammation related cytokines and chemokines were detected in liver.

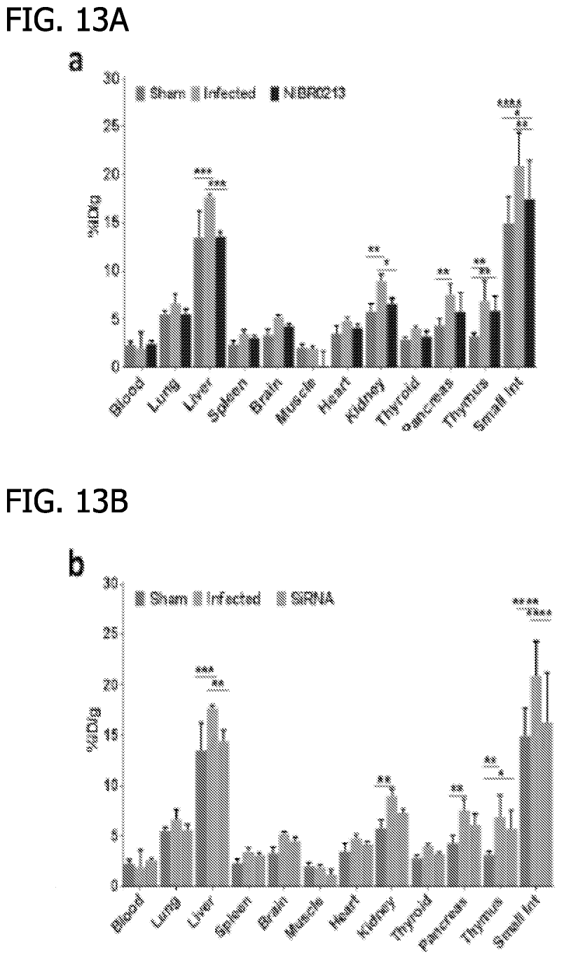

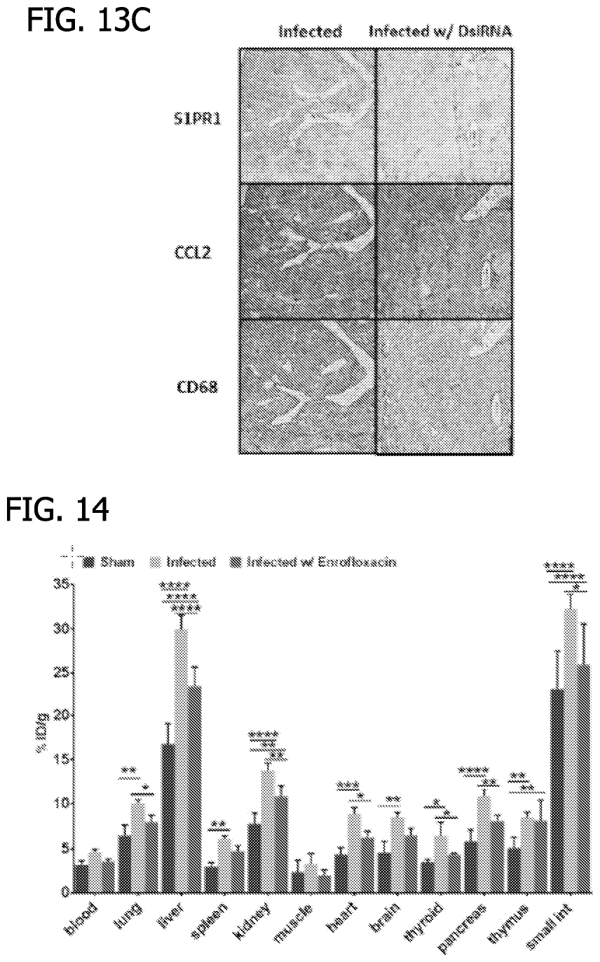

NIBR-0213 (S1P1 inhibitor; 5 mg/kg) (FIG. 13A) or DsiRNA treatment (20 μg/kg) (FIG. 13B) was administered 12 hrs prior to tracer injection, and tissue uptake of [¹⁸F]TZ4877 was measured. The infected mice livers have higher S1P1, CD68, and CCL2 expression compared to DsiRNA pretreatment (FIG. 13B). IHC staining was used for confirmation (FIG. 13C).

Enrofloxacin is an antibiotic drug, used for anti-inflammation in clinic practice. Enrofloxacin (5 mg/kg) was administered 12 hrs prior to inoculation and 24 hrs prior to tracer injection, and tissue uptake of [¹⁸F]TZ4877 was measured (FIG. 14).

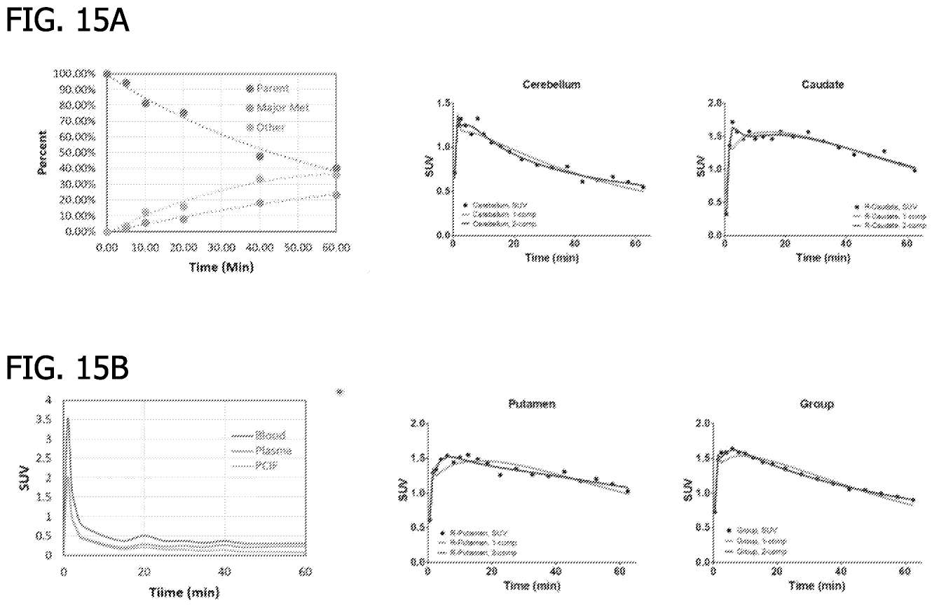

Additional uptake of ¹⁸F-TZ48-77 in non-human primates was also performed. The percentage of the parent compound compared to the major metabolite and other compounds was measured, including in blood, plasma, and PCIF (FIG. 15A). The standardized uptake value was also calculated for various brain regions (FIG. 15B).

### Example 9: Autoradiography Studies of Human and Rat Brain Tissues Using [³H]TZ3321

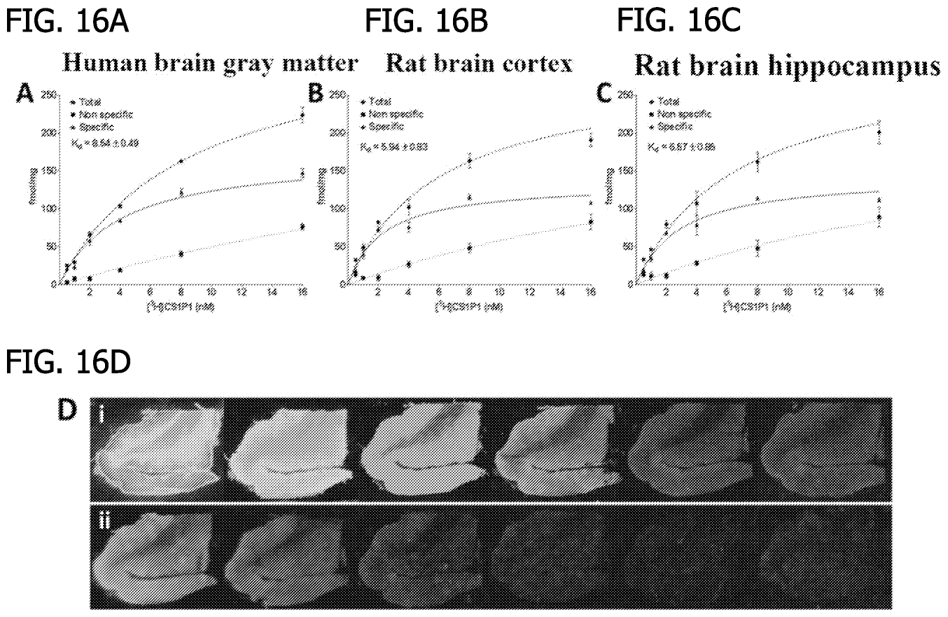

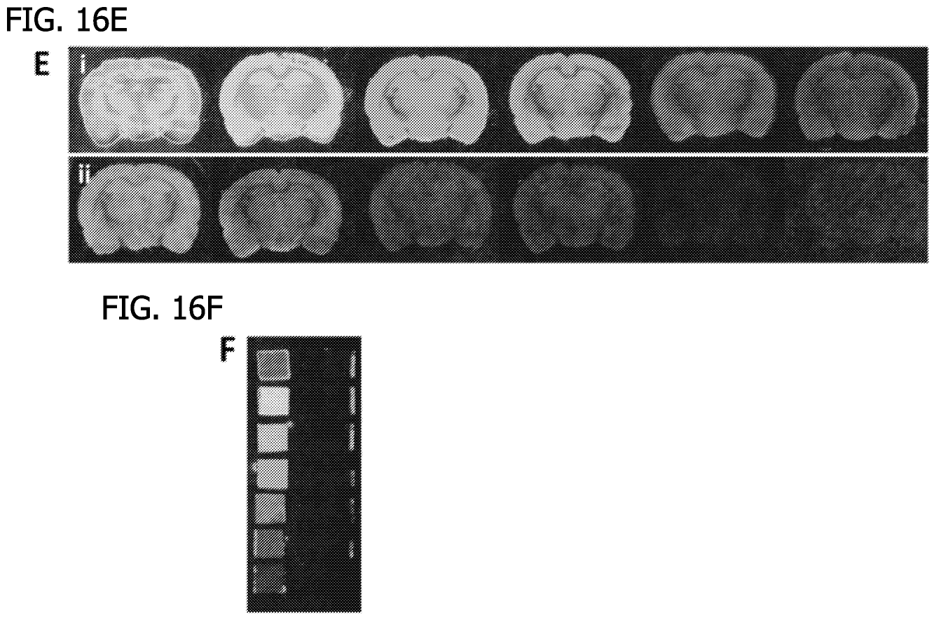

In vitro saturation binding autoradiograph analysis of [³H]CS1P1 was performed. A high affinity of [³H]CS1P1 to the gray matter of human frontal cortex was found with a Kd of 8.54 nM (FIG. 16A). A high affinity of [³H]CS1P1 to the cortex region of rat brain with a Kd of 5.94 nM (FIG. 16B). A high affinity of [³H]CS1P1 to the hippocampus region of rat brain with a Kd of 6.57 nM (FIG. 16C). Representative images of [³H]CS1P1 in human frontal cortex (FIG. 16D), [³H]CS1P1 in rat brain (FIG. 16E), and tritium ART-123 standard (FIG. 16F) are also presented.

### Example 10: Radiosynthesis of Three [18F] Fluoroethoxyl Radiotracers

This example involves exploration of [18F]TZ4877 derivatives to optimize the radiopharmaceutical properties of the S1P1 radiotracer.

The reagents and conditions for the below syntheses are as follows: a) ethylene ditosylate, K₂CO₃, CH₃CN, 90° C.; b) [¹⁸F]KF, Kryptofix 222, K₂CO₃, CH₃CN, 110° C., 15 min, 6 N HCl, 5 min. ethylene ditosylate, K₂CO₃, CH₃CN, 90° C.; b) [¹⁸F]KF, Kryptofix 222, K₂CO₃, CH₃CN, 110° C., 15 min, 6 N HCl, 5 min.

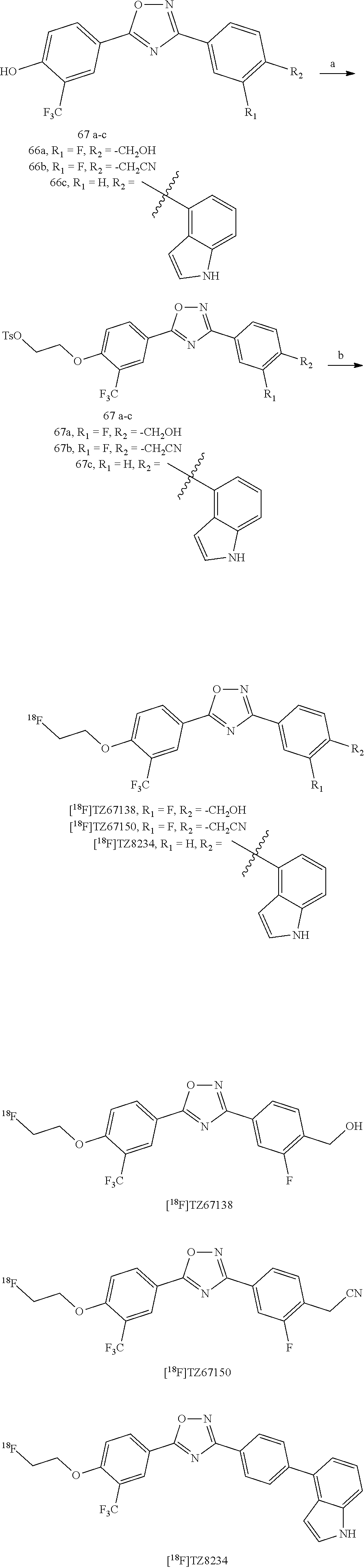

### Example 11: Radiosynthesis of Six [18F] Fluoroaryl Containing Radiotracers

For below, the reagents and conditions are: (a) 1) methyl 3-aminopropanoate, methyl glycinate, or methyl 4-aminobutanoate, AcOH, EtOH, 100° C., 5 min, 2) NaCNBH₃, RT, 2 min, 3) formalin, NaCNBH₃, 100° C., 5 min, 4) NaOH (5 M), 100° C., 5 min; (b) NaBH₄, EtOH, RT, 2 min; (c) 1) FeCl₃, 2,2,7,7-tetramethyl-3,6-dioxa-2,7-disilaoctane, MeCN, 100° C., 5 min, 2) Et₃SiH, MeCN, 100° C., 5 min; (d)) FeCl₃, 2,2,10,10-tetramethyl-3,6,9-trioxa-2,10-disilaundecane, MeCN, 100° C., 5 min, 2) Et₃SiH, MeCN, 100° C., 5 min.

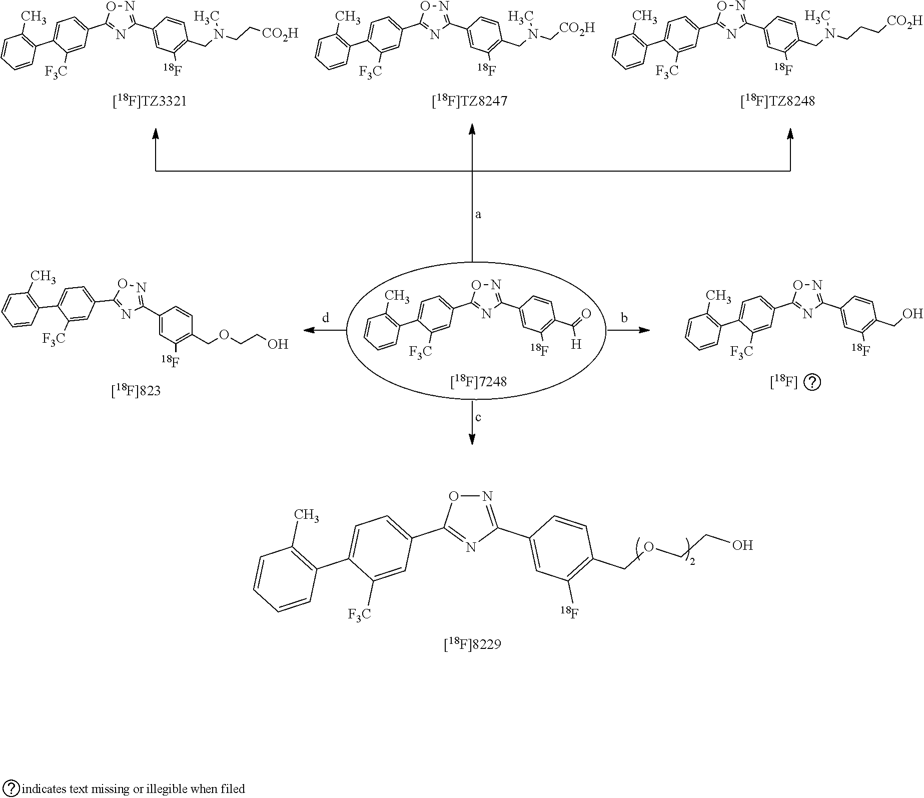

### Example 12: Biodistribution Data of Additional F-18 S1P1 Radiotracers

Sphingosine 1-phosphate receptor 1 (S1P1) has high expression under many neuroinflammatory conditions, and especially in multiple sclerosis (MS) disease. Positron emission tomography (PET) imaging targeting S1P1 is able to quantify the S1P1 expression level, and then provide important neuroinflammatory activity information in the central nervous system (CNS). Here, second-generation S1P1 specific F-18 labeled tracers from [¹⁸F]FS1P1 were explored and initially evaluated in nonhuman primate.

The S1P1 ligands were synthesized using conventional reaction conditions with necessary modification. In vitro binding affinities were determined by competitive S1Ps cell membrane assay against the radioligand [³²P]S1P. Three compounds were identified (TZ8247, TZ8248, and TZ823) that have high specific binding toward S1P1 with IC₅₀ less than 20 nM. The radiosynthesis of [¹⁸F]TZ8247 and [¹⁸F]TZ8248 was carried out by a published protocol of [¹⁸F]FS1P1 with different amino esters. The radiosynthesis of the PEGylated tracer [¹⁸F]TZ823 was realized by employing an acetal [¹⁸F]3 intermediate. After [¹⁸F]nucleophilic aromatic substitution of nitro group, the [¹⁸F]fluorobenzaldehyde 2 was transferred into [¹⁸F]acetal 3 by 2,2,7,7-tetramethyl-3,6-dioxa-2,7-disilaoctane under the catalysis of iron(III) chloride, followed by reduction with triethylsilane to offer the target radioligand [¹⁸F]TZ823. The brain uptakes of these tracers were measured by Focus 220 microPET scanner to collect dynamic scans for 2 hours after intravenous injection of ˜0.35 GBq dose into male cynomolgus macaques (8-9 kg).

12 fluorous compounds were synthesized in moderate to good yield, and 8 were tested for uptake. The compounds tested are shown below:

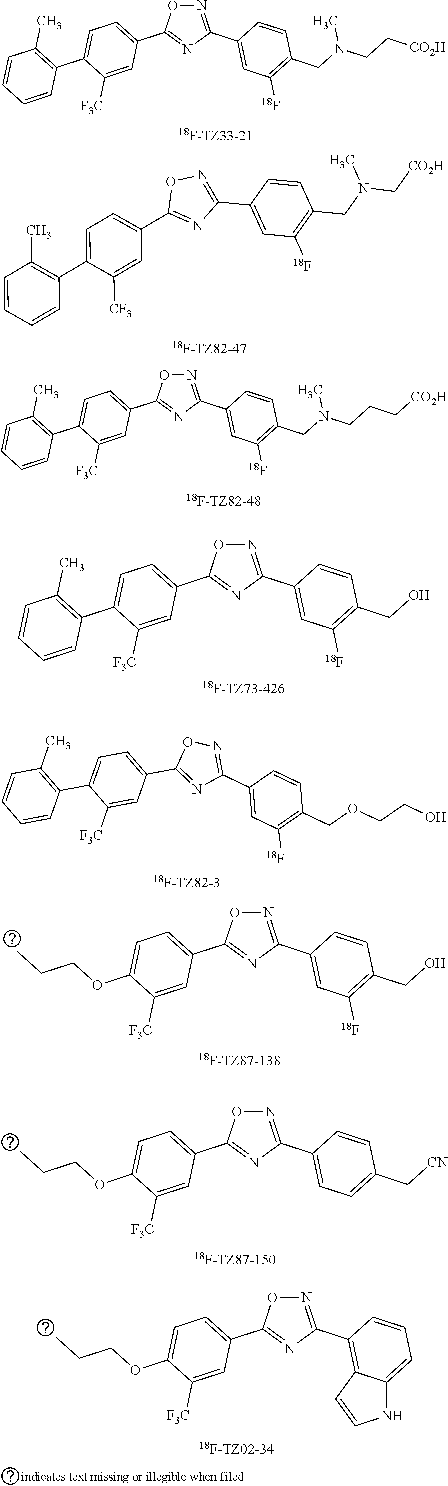

The synthesis is shown below:

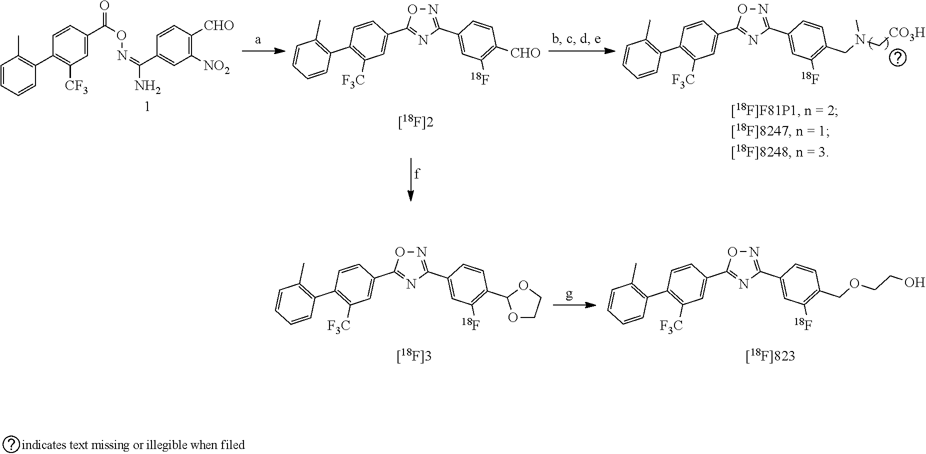

For this synthesis, the reagents and conditions are as follows. A) [¹⁸F]KF, Kryptofix 222, TMEDA, DMSO/H₂O, 150° C., 5 min. B) β-alanine methyl ester, methyl glycinate, or methyl 4-aminobutanoate, AcOH, EtOH, 100° C., 5 min. C) NaCNBH₃, RT, 2 min. D) Formalin, 100° C., 5 min, then NaCNBH₃, RT, 2 min. e) NaOH (5 M), 100° C., 5 min, then AcOH for neutralization. F) FeCl₃, 2,2,7,7-tetramethyl-3,6-dioxa-2,7-disilaoctane, MeCN, 100° C., 5 min. G) FeCl₃, Et₃SiH, MeCN, 100° C., 5 min.

Another layout of this synthesis is shown below.

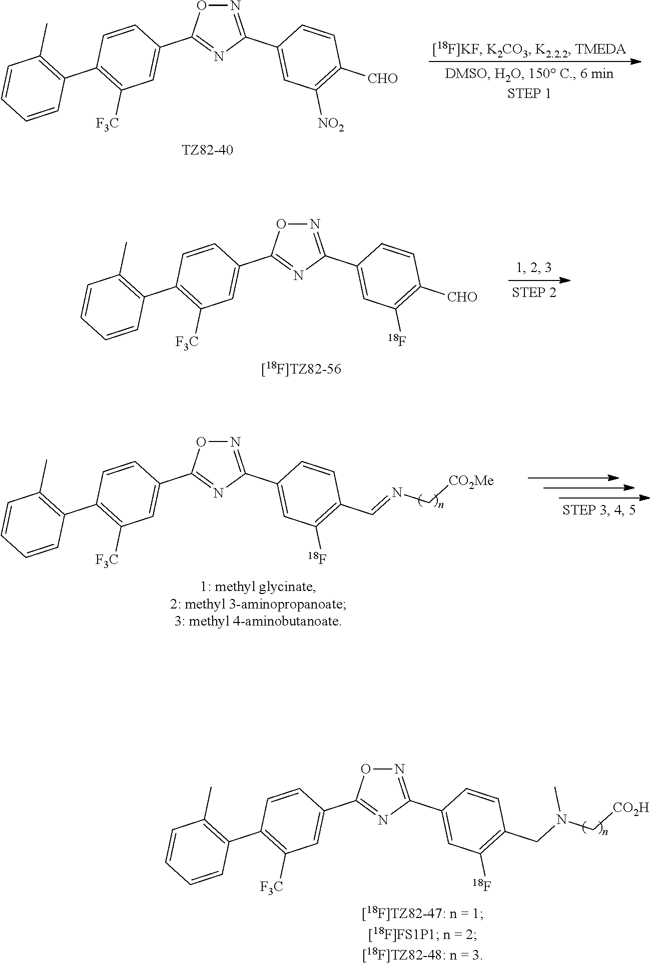

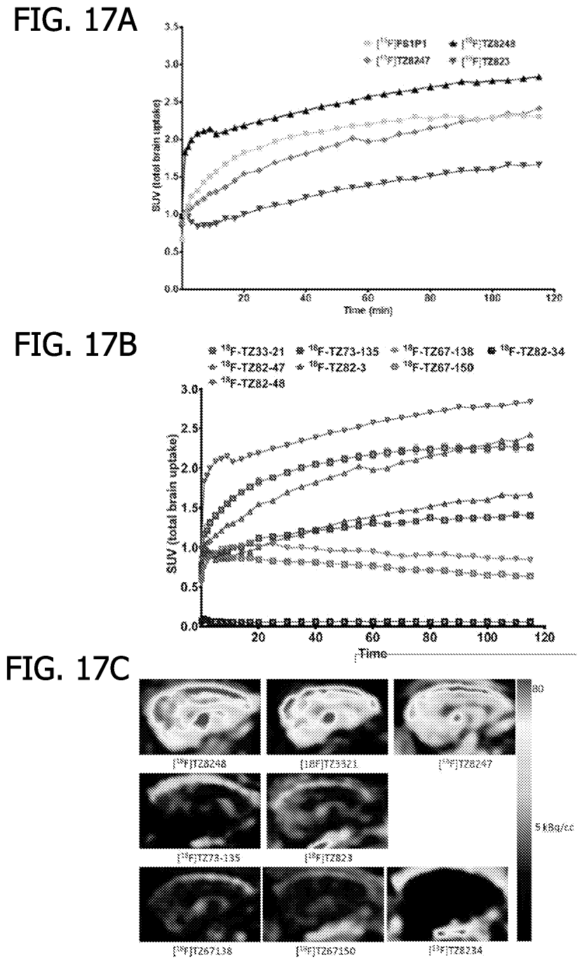

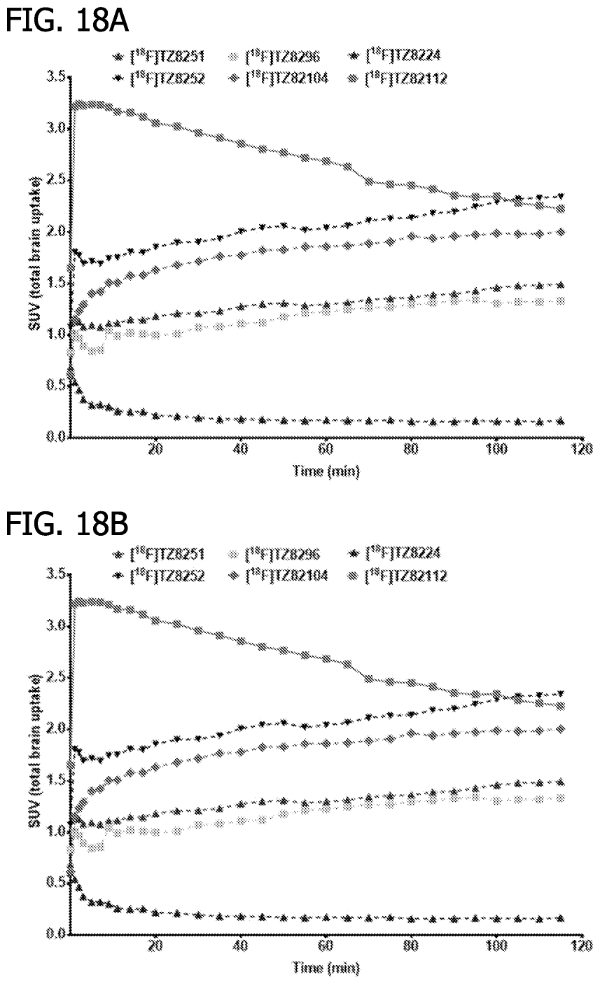

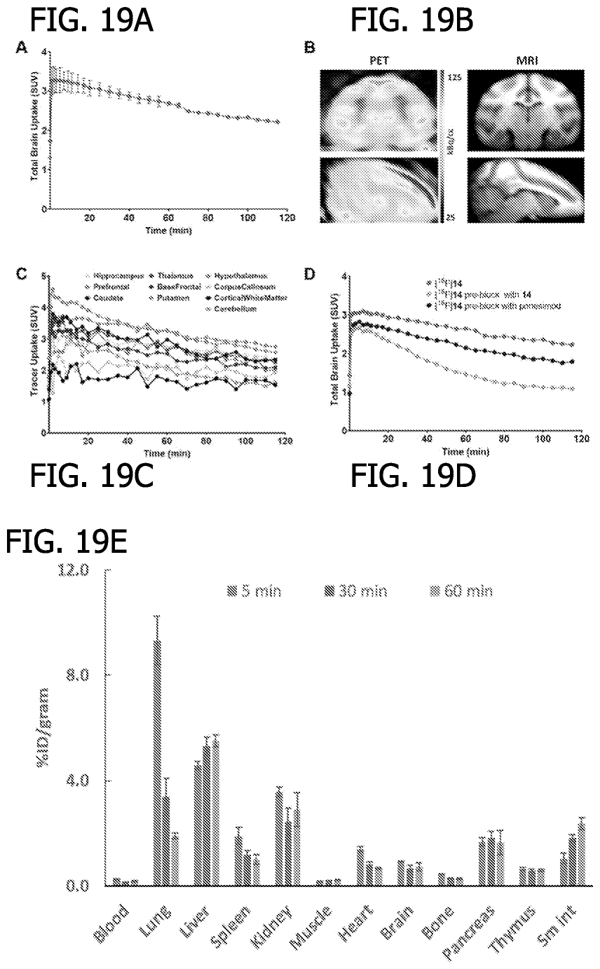

The binding assay results showed TZ8247, TZ8248, and TZ823 have high binding affinity for S1P1 with IC₅₀ values of 7.6±1.6 nM, 0.8±0.7 nM, and 12.3±2.2 nM, respectively, and good selectivity over S1P2-5. The radiosynthesis of [¹⁸F]TZ8247, [¹⁸F]TZ8248, and [¹⁸F]TZ823 was achieved with good radiochemical yield (20˜25%), high radiochemical purity (>95%), and high molar activity (>40 GBq/μmol, EOB). NHP microPET scans revealed that both [¹⁸F]TZ8247 and [¹⁸F]TZ8248 with carboxylic acid tail and ˜2.8, respectively at 120 min post-injection (FIG. 17A and FIG. 17B). However, the brain uptake of PEGylated tracer [¹⁸F]TZ823 is low to ˜1.6 at 120 min post-injection. Representative scans are also presented (FIG. 17C).

A series of S1P1 specific analogues was synthesized and characterized. The in vitro competitive binding affinity assay showed 3 compounds TZ8247, TZ8248, and TZ823 possessed high binding potency toward S1P1 (IC₅₀<20 nM) and good selectivity over S1P2-5. The radiosynthesis of [¹⁸F]TZ8247 and [¹⁸F]TZ8248 was achieved with good radiochemical yields and purity. The PEGylated tracer [¹⁸F]TZ823 was also radio-labeled by introducing a key acetal [¹⁸F]3 intermediate with good results.

The MicroPET data suggested that both [¹⁸F]TZ8247 and [¹⁸F]TZ8248 had high brain uptake in nonhuman primates.

### Example 13: S1P1 Ligand and Binding Potency

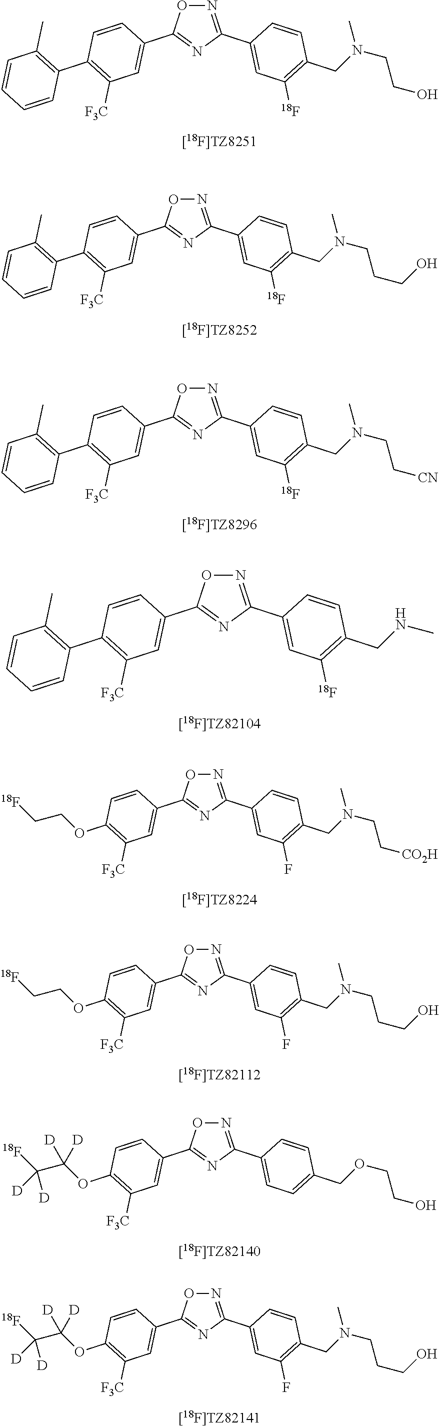

Our PET imaging studies with [¹⁸F]TZ4877 to investigate the S1PR1 expression response in infection induced by Staphylococcus aureus (S. aureus) bacterial in rodent model demonstrated that PET with S1PR1 radiotracer has high possibility to be a biomarker for assessing the infectious status. The manuscript of this study was published in this progress report year.

Our NHP PET brain imaging blocking studies showed that pretreatment with cold TZ4877 or TZ82112 can reduce the brain uptake of [¹⁸F]4877, indicating that [¹⁸F]4877 in the brain is S1PR1 specific. For radiotracer [¹⁸F]TZ82112, pretreated with cold TZ82112 is able to reduce the its brain uptake of the animal. Together, our data demonstrated both [¹⁸F]TZ4877 and [¹⁸F]TZ82112 are able to specifically bind to the S1PR1 in the brain and are S1PR1-specific radioligands.

Ex-vivo rat biodistribution studies showed [18F]TZ82112 has a high initial brain uptake and good brain washout kinetics. PET studies that the EAE rat model of MS has increased spinal cord uptake of [¹⁸F]82112 compared to the normal controls, however, the increase percentage is not as higher as other promising S1PR1 radiotracers such as [¹⁸F]TZ4877 or [¹⁸F]FCS1P1/[¹¹C]CS1P1.

HPLC radiometabolite analysis of rat plasma and nonhuman primate plasma samples found that although [¹⁸F]TZ82112 quickly generated a high percentage of a radiometabolite that is more lipophilic than the parent radiotracer in rat plasma, but the percentage of that same metabolite was much lower in NHP plasma, indicating the metabolism of [¹⁸F]TZ82112 has species difference. Metabolite analysis of the rat brain tissue samples found no radioactive metabolite in the rat brain, suggesting that radiometabolite will impact the ability of PET with [¹⁸F]TZ82112 to precisely measure of S1PR1 expression in the brain for neuroinflammation. We believe [¹⁸F]TZ82112 will be more stable in the human plasma than in rats although that will need to be confirmed from HPLC metabolite analysis of human plasma samples post-injection of the tracer. Considering the high NHP brain uptake and clinically favorable pharmacokinetics compared to other F-18 S1PR1 radiotracers such as [¹⁸F]FCS1P1 and [¹⁸F]4877, we believe [¹⁸F]TZ82112 is worth to transfer into clinical evaluation to confirm [18F]TZ82112 is the best S1PR1 radiotracer for clinical assessment of the neuroinflammation status by measurement of the S1PR1 receptor expression level. On the other side, as a S1PR1 radiotracer, [¹⁸F]TZ82112 has its limitation for preclinical investigation because it quickly metabolites to form a lipophilic radiometabolite which will increase noise signal for PET measurement of S1PR1 expression in inflamed tissues.

The following compounds can be prepared using the following general synthetic schemes.

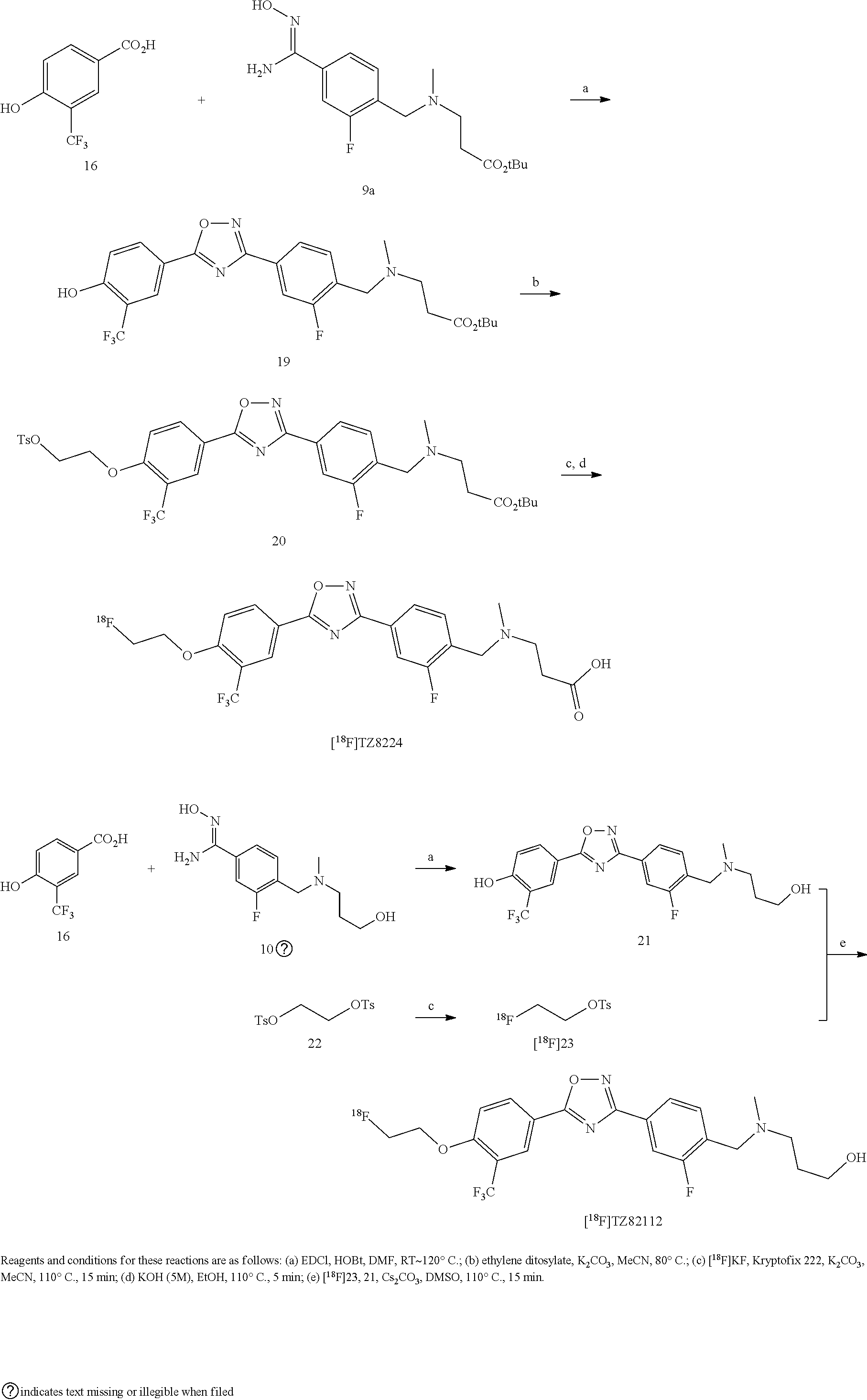

Deuterated analogs of the compounds can be prepared using the following general reaction scheme.

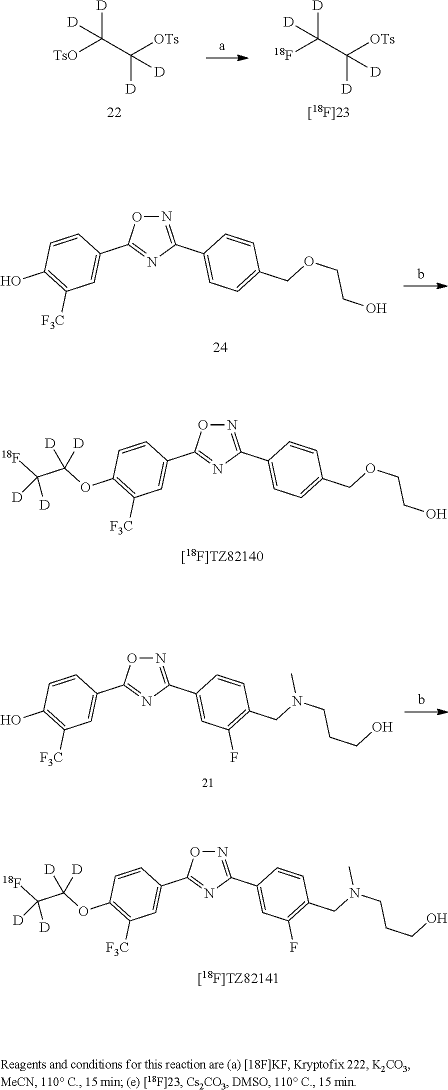 [SMILES: [2H]C([2H])(OS(=O)(=O)c1ccc(C)cc1)C([2H])([2H])OS(=O)(=O)c1ccc(C)cc1 ; [2H]C([2H])([18F])C([2H])([2H])OS(=O)(=O)c1ccc(C)cc1 ; OCCOCc1ccc(-c2noc(-c3ccc(O)c(C(F)(F)F)c3)n2)cc1 ; [2H]C([2H])([18F])C([2H])([2H])Oc1ccc(-c2nc(-c3ccc(COCCO)cc3)no2)cc1C(F)(F)F ; CN(CCCO)Cc1ccc(-c2noc(-c3ccc(O)c(C(F)(F)F)c3)n2)cc1F ; [2H]C([2H])([18F])C([2H])([2H])Oc1ccc(-c2nc(-c3ccc(CN(C)CCCO)c(F)c3)no2)cc1C(F)(F)F]

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. A compound having a structure of Formula (I) or (II), a pharmaceutically acceptable salt thereof, or a prodrug thereof: 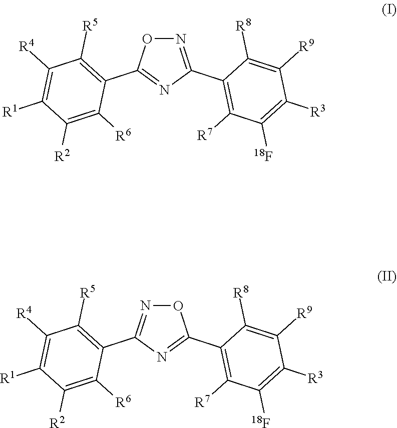
wherein
R¹ is substituted or unsubstituted aryl or substituted or unsubstituted alkoxy;
R² is C₁-C₄ haloalkyl or cyano;
R³ and R⁹ are each independently hydrogen, halo, hydroxy, substituted or unsubstituted amino, substituted or unsubstituted sulfonyl, substituted or unsubstituted hydrocarbyl;
R⁴ are each independently hydrogen, substituted or unsubstituted C₁-C₆ alkyl, or substituted or unsubstituted C₁-C₆ alkoxy; and
R⁵, R⁶, R⁷ and R⁸ are each independently hydrogen, halo, hydroxy, substituted or unsubstituted C₁-C₆ alkyl, or substituted or unsubstituted C₁-C₆ alkoxy.

2. The compound of claim 1 wherein R¹ is substituted or unsubstituted aryl.

3. A compound having a structure of Formula (V), or a pharmaceutically acceptable salt or a prodrug thereof: 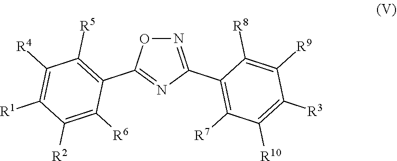
wherein:
R¹ is substituted or unsubstituted alkoxy;
R² is C₁-C₄ haloalkyl or cyano;
R³ and R⁹ are each independently hydrogen, halo, hydroxy, substituted or unsubstituted amino, substituted or unsubstituted sulfonyl, substituted or unsubstituted hydrocarbyl;
R⁴ are each independently hydrogen, substituted or unsubstituted C₁-C₆ alkyl, or substituted or unsubstituted C₁-C₆ alkoxy;
R⁵, R⁶, R⁷, and R⁸ are each independently hydrogen, halo, hydroxy, substituted or unsubstituted C₁-C₆ alkyl, or substituted or unsubstituted C₁-C₆ alkoxy; and
R¹⁰ is hydrogen or fluorine.

4.-6. (canceled)

7. The compound of claim 3 wherein R¹ or R⁴ is a —OCH₂CH₂F or —OCH₂CH₂F¹⁸.

8. The compound of claim 1 wherein R⁴ is hydrogen or a C₁-C₆ alkyl.

9. The compound of claim 1 wherein R¹ is C₁-C₆ alkyl substituted phenyl.

10. The compound of claim 1 wherein R² is —CF₃.

11. The compound of claim 1 wherein at least one of R³ or R⁹ is substituted or unsubstituted C₁-C₆ alkyl or substituted or unsubstituted C₁-C₆ alkoxy.

12. The compound of claim 1 wherein at least one of R³ or R⁹ is —CH₂(OCH₂CH₂)ₙOH or —(OCH₂CH₂)ₙOH, where n is from 0 to 10, from 0 to 8, from 0 to 6, from 0 to 4, or from 0 to 2.

13. (canceled)

14. The compound of claim 1 wherein at least one of R³ or R⁹ is —CH₂R¹¹ where R¹¹ is substituted alkoxy, substituted or unsubstituted amino, a substituted or unsubstituted amido, an azide, a substituted or unsubstituted sulfonyl, a substituted sulfur-containing ring, or a substituted or unsubstituted nitrogen-containing ring.

15. The compound of claim 1 wherein at least one of R³ or R⁹ is

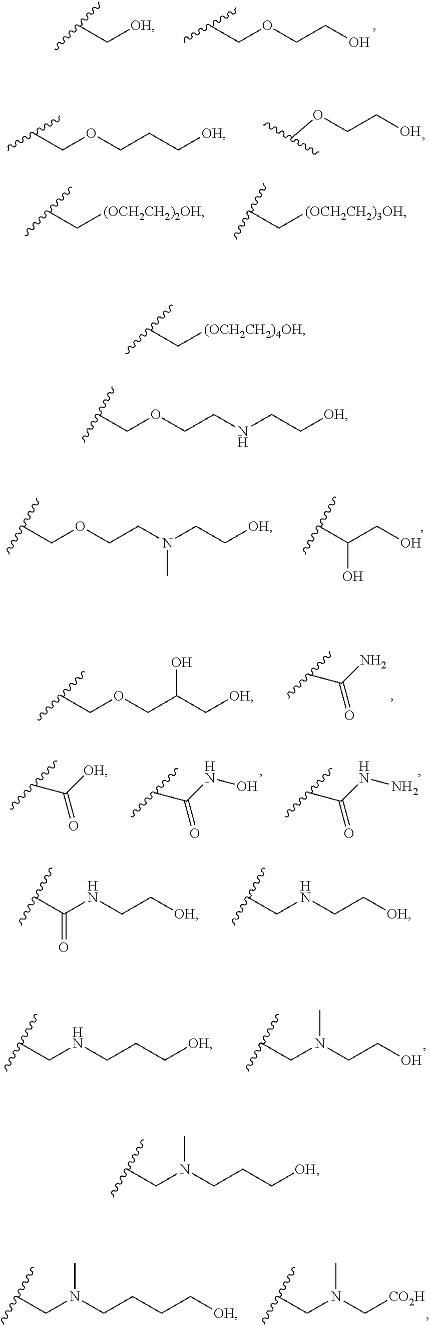

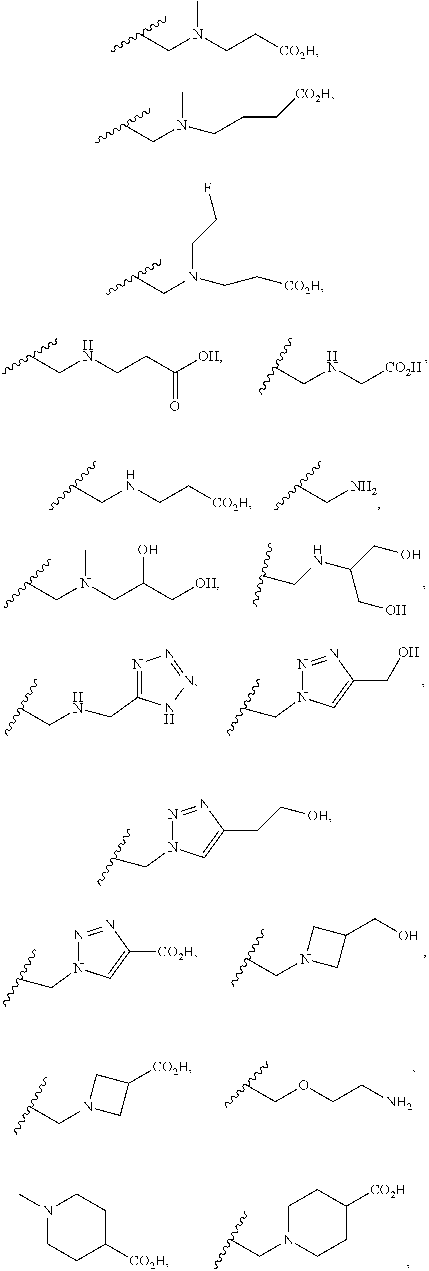

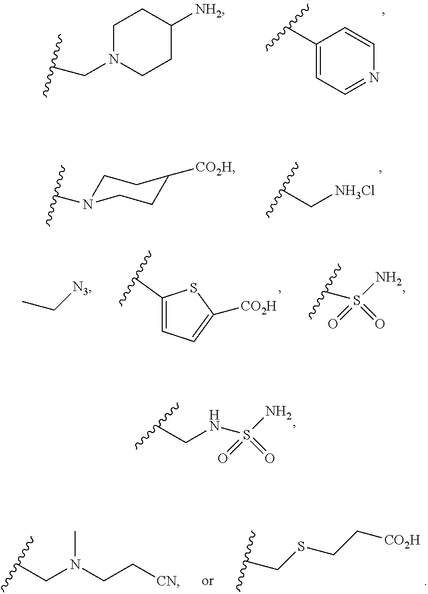

16. The compound of claim 1 wherein at least one of R³ or R⁹ is —CH(OH)CH₂OH.

17.-18. (canceled)

19. The compound of claim 1 wherein R⁵, R⁶, R⁷, R⁸, and R⁹ are each independently hydrogen.

20.-22. (canceled)

23. The compound of claim 1 wherein Formula (I) has a structure selected from the group consisting of: 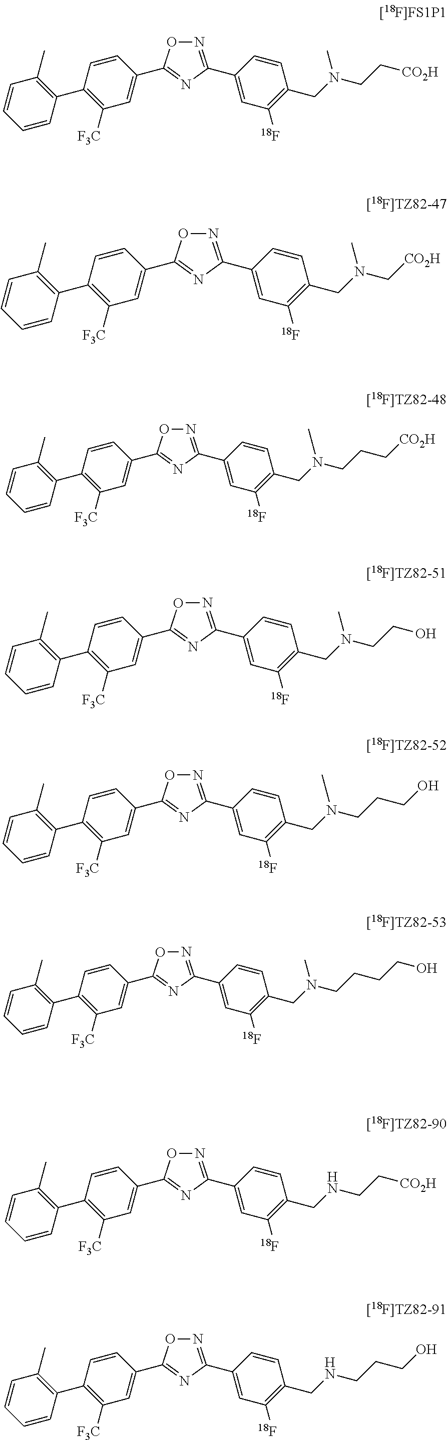 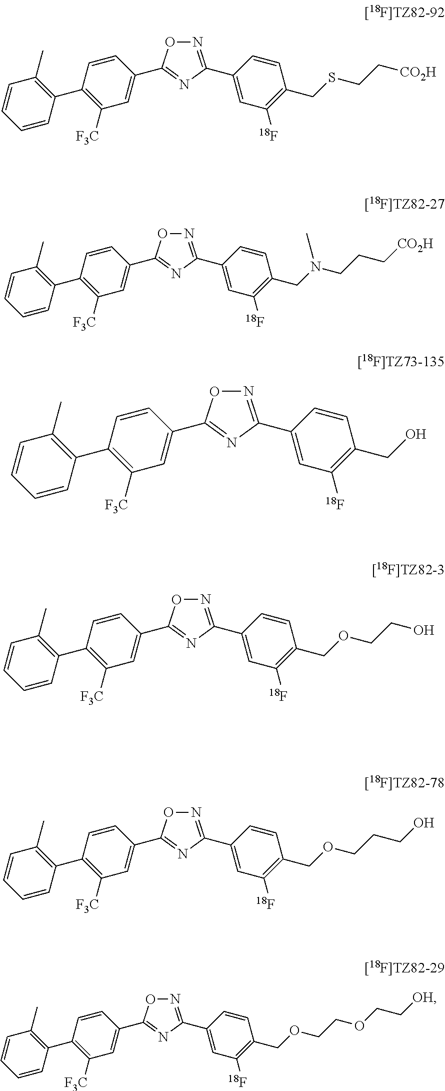 [SMILES: Cc1ccccc1-c1ccc(-c2nc(-c3ccc(CSCCC(=O)O)c([18F])c3)no2)cc1C(F)(F)F ; Cc1ccccc1-c1ccc(-c2nc(-c3ccc(CN(C)CCCC(=O)O)c([18F])c3)no2)cc1C(F)(F)F ; Cc1ccccc1-c1ccc(-c2nc(-c3ccc(CO)c([18F])c3)no2)cc1C(F)(F)F ; Cc1ccccc1-c1ccc(-c2nc(-c3ccc(COCCO)c([18F])c3)no2)cc1C(F)(F)F ; Cc1ccccc1-c1ccc(-c2nc(-c3ccc(COCCCO)c([18F])c3)no2)cc1C(F)(F)F ; Cc1ccccc1-c1ccc(-c2nc(-c3ccc(COCCOCCO)c([18F])c3)no2)cc1C(F)(F)F]
pharmaceutically acceptable salts thereof, prodrugs thereof, and mixtures thereof.

24. The compound of claim 3 wherein Formula (V) has a structure selected from the group consisting of:

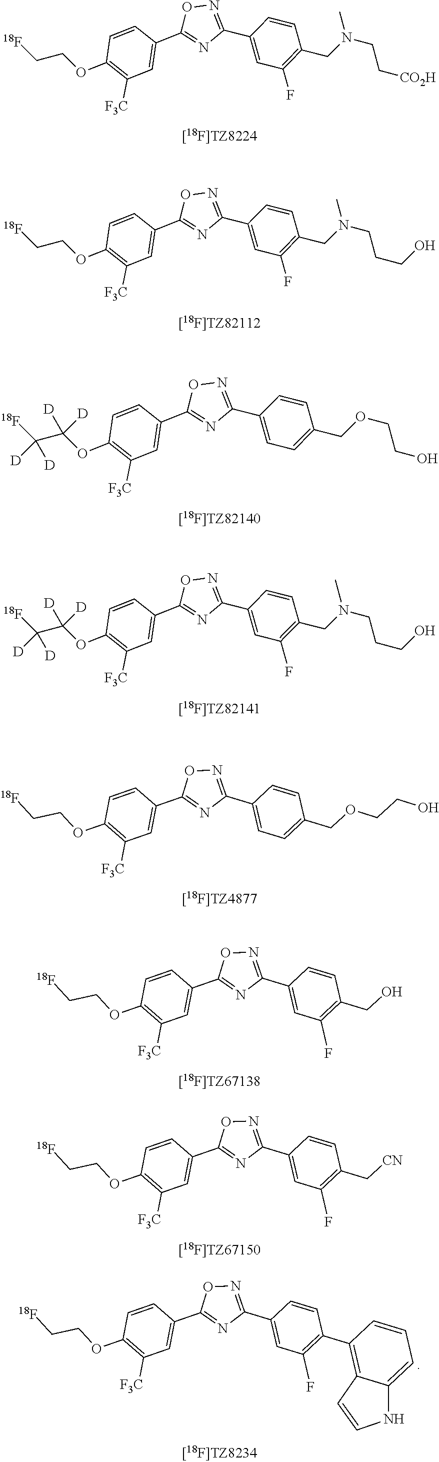

25.-27. (canceled)

28. A pharmaceutical composition comprising the compound of claim 1 and at least one excipient or carrier.

29. (canceled)

30. A method of diagnosing or monitoring an S1P1 associated disease, disorder or condition in a mammal comprising administering a compound of claim 1 to the mammal; and detecting the compound in the mammal.

31.-32. (canceled)

33. A method of quantifying S1P1 expression in a mammalian brain or central nervous system comprising administering a compound of claim 1 to the mammal; and detecting the compound in the mammal.

34. (canceled)

35. A method of treating an S1P1 associated disease, disorder, or condition in a subject in need thereof, comprising administering to the subject a pharmaceutical composition comprising a therapeutically effective amount of an S1P1 modulating agent comprising a compound of claim 1, and inhibiting, slowing the progress of, or limiting the development of the S1P1 associated disease, disorder, or condition.

36. (canceled)

37. A process for preparing a compound of claim 1, the process comprising:
reacting a [¹⁸F] fluoride salt with a precursor compound of formula (III) or (IV) 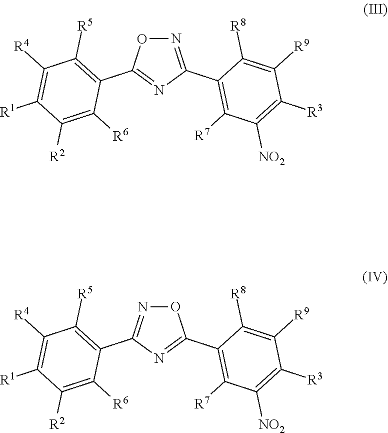
to form a [¹⁸F] radiolabeled precursor.
